(19) United States
(12) Patent Application Publication
(10) Pub. No.: US 2023/0287389 A1
(43) Pub. Date: Sep. 14, 2023

(54) A METHOD FOR GENERATING NEW MUTATION IN ORGANISM AND USE THEREOF

(71) Applicant: Qingdao Kingagroot Chemical Compound Co., Ltd., Qingdao (CN)

(72) Inventors: Linjian JIANG, Qingdao (CN); Sudong MO, Qingdao (CN); Jiyao WANG, Qingdao (CN); Yucai LI, Qingdao (CN); Wei QI, Qingdao (CN); Huarong LI, Qingdao (CN); Bo CHEN, Qingdao (CN)

(21) Appl. No.: 17/058,261

(22) PCT Filed: Oct. 13, 2020
(86) PCT No.: PCT/CN2020/120633

§ 371 Date: Nov. 24, 2020

(30) Foreign Application Priority Data
Nov. 7, 2019 (CN) 201911081617.X
Aug. 15, 2020 (CN) 202010821877.2
Sep. 16, 2020 (CN) 202010974151.2

(51) Int. Cl. C12N 15/10; C12N 15/65; C12N 15/82

(52) U.S. Cl. CPC C12N 15/102; C12N 15/65; C12N 15/8274; C12N 15/8213; C12N 2310/20; C12N 2510/00

(57) ABSTRACT

The present invention pertains to the technical field of genetic engineering, and specifically relates to a method for generating a site-specific mutation in an organism in the absence of an artificial DNA template and a use thereof. The method comprises the following steps: sequentially generating two or more DNA breaks at a specific site in a genome of an organism and spontaneously repairing them respectively, wherein a later DNA break is generated based on a new sequence generated from a previous DNA break repair. In the present invention, a new target is designed based on a sequence formed by a new repair event generated by sequential editing, and thus, mutations may be sequentially formed for multiple times at a specific site in the genome, which greatly enrich the types of repair events after DNA breaks, and realize new base substitution, deletion and insertion mutations that cannot be obtained in a single gene editing.

### CROSS-REFERENCE TO RELATED APPLICATIONS

This application is a national stage filing under 35 U.S.C. § 371 of International Application No. PCT/CN2020/120633, filed Oct. 13, 2020, and claims the priority to and benefits of Chinese Patent Application No. 201911081617.X, filed Nov. 7, 2019, Chinese Patent Application No. 202010821877.2, filed Aug. 15, 2020, and Chinese Patent Application No. 202010974151.2, filed Sep. 16, 2020, which are incorporated herein by reference in their entireties.

### SEQUENCE LISTING

This application contains a Sequence Listing electronically submitted via EFS-Web to the United States Patent and Trademark Office as an ASCII text file entitled “15500_0005-00155_SL.txt” having a size of 178,681 bytes and created on Nov. 17, 2021. The information contained in the Sequence Listing is incorporated by reference herein.

### TECHNICAL FIELD

The present invention pertains to the technical field of genetic engineering, and specifically relates to a method for generating a site-specific mutation in an organism in the absence of an artificial DNA template and a use thereof.

### BACKGROUND ART

The genetic engineering technology for modifying genome of organisms has been widely used in industrial and agricultural production, such as genetically modified microorganisms commonly used in the pharmaceutical and chemical fields, and genetically modified crops with insect-resistance and herbicide-resistance in the agricultural field. With the advent of site-specific nucleases, by introducing a targeted fragmentation into the genome of recipient organism and causing spontaneous repair, it has been possible to achieve site-specific editing of genome and more precise modification of genome.

Gene editing tools mainly include three types of sequence-specific nuclease (SSN): zinc finger nuclease (ZFN), transcription activator-like effector nuclease (TALEN) and clustered regularly interspaced short palindromic repeats (CRISPR) associated Cas system (CRISPR/Cas system). Sequence-specific nucleases are programmable nucleases that can generate DNA double-strand breaks (DSBs) at specific sites in genome. DNA double-strand breaks activate the endogenous DNA repair pathway to repair DNA damage in a cell, but the repair process easily leads to changes in the DNA sequence at target sites, thereby achieving the introduction of mutations at interesting sites. This technology enables biologists to accurately target a target gene and edit it. Among them, both ZFN and TALEN need to design specific recognition protein modules for the target sequence, thereby having low throughput and complex operations. However, Cas protein is universal in the CRISPR/Cas system, in which a guide RNA (gRNA) can be formed by a specific CRISPR-RNA (crRNA) designed for a target site alone or in conjunction with transactivating RNA (tracrRNA), or a single guide RNA (sgRNA) alone is enough, the crRNA and tracrRNA together or sgRNA alone can be assembled with the Cas protein to form a ribonucleoprotein complex (RNP), the target sequence is identified on the basis of protospacer adjacent motif (PAM) in genome, thereby realizing site-specific editing. And thus, it has become a main gene editing tool because of its simple operation, wide application range and high throughput.

Sequence-specific nuclease can produce DNA double-strand breaks at specific sites in the genome. These DNA double-strand breaks can be repaired into a variety of different repair types, which are mainly base insertions or deletions. For example, the two most common types of CRISPR/Cas9 editing events are inserting a base at the break or deleting a base at the break (Shen et al. 2018. Predictable and precise template-free CRISPR editing of pathogenic variants. Nature. DOI: 10.1038/s41586-018-0686-x). The insertion or deletion of bases in coding region will cause frameshift mutations, leading to loss of gene function. Therefore, the main purpose of the above gene editing tools is still to perform gene knockout.

It has been considered all the time that the use of sequence-specific nuclease alone cannot achieve mutations in type of base substitution. To this end, the prior art proposes three solutions: 1) adding an exogenous DNA fragment as a repair template to initiate a homologous recombination repair pathway; 2) fusing deaminase with Cas9 to sequentially develop single-base editing tools for C to T and A to G; 3) fusing reverse transcriptase with Cas9, using pegRNA to guide the synthesis and substitution of small DNA strand. However, the editing efficiency of these three solutions is significantly lower than the efficiency of gene knockout, and the simultaneously introduced exogenous DNA fragment and reverse transcriptase may readily cause concerns about biological safety. The off-target effect of single-base editing also restricts its potential application in cell therapy. Especially for long-term plant breeding projects, how to improve base substitution efficiency at target site while reducing the regulatory authorities' concerns about biosafety is a problem that needs to be resolved in the application of gene editing technology.

In summary, there are urgent technical needs in the fields of cell therapy and biological breeding for site-specific base substitution by using only targeted knockout of sequence-specific nuclease without introducing a foreign DNA fragment, especially through the non-transgenic transient editing system to efficiently complete site-specific base substitution editing.

### SUMMARY OF INVENTION

The invention provides a method for generating a site-specific mutation in an organism only by generating double-strand breaks on a genome and without providing an artificial DNA template, and use of the method.

The technical solutions adopted by the present invention are as follows:

A method for generating a new mutation in an organism, which comprises the following steps: sequentially generating two or more DNA breaks at a specific site in a genome of the organism and spontaneously repairing them respectively, wherein a later DNA break is generated based on a new sequence generated from a previous DNA break repair.

In a specific embodiment, the “DNA break” is achieved by delivering a nuclease with targeting property into a cell of an organism to contact with a specific site of genomic DNA.

In a specific embodiment, the “nuclease with targeting property” is a ZFN, TALEN or CRISPR/Cas system.

In a specific embodiment, the “sequentially generating two or more DNA breaks at a specific site” refers to that based on a new sequence generated from a previous DNA break repair event caused by ZFN or TALEN editing, a new ZFN or TALEN protein is designed to cut the site again.

In another specific embodiment, “sequentially generating two or more DNA breaks at a specific site” refers to that based on a new sequence generated from a previous DNA break repair event caused by a CRISPR/Cas system, a new target RNA is designed to cut the site again. For example, a second cutting is performed at the site again by designing a new target RNA on the basis of a new sequence generated from a first break repair event of Cas9 editing. In a similar way, a third cutting is performed at the site by designing a new target RNA on the basis of a new sequence generated from a second break repair event and so on, as shown in FIG. 1.

In a specific embodiment, the “two or more DNA breaks” are generated by sequentially delivering different targeted nucleases into recipient cells of different generations, wherein a mutant cell that has completed the previous editing is used as a recipient to receive the delivery of the targeted nuclease for the later editing, thereby performing second editing to generate site-specific mutation. This method is preferably used for ZFN and TALEN editing systems.

In another specific embodiment, the “two or more DNA breaks” are generated by delivering different targeted nucleases for different targets into a same recipient cell. This method is preferably used for CRISPR/Cas editing system.

In a specific embodiment, the “two or more DNA breaks” are generated when RNP complexes formed by a same CRISPR/Cas nuclease respectively with different gRNAs or sgRNAs sequentially cut corresponding target sequences.

In another specific embodiment, the “two or more DNA breaks” are generated when RNP complexes formed by each of two or more CRISPR/Cas nucleases that recognize different PAM sequences with respective gRNA or sgRNA, sequentially cut corresponding target sequences. For example, the PAM sequence recognized by Cas9 from Streptococcus pyogenes is “NGG” or “NAG” (Jinek et al., “A programmable dual-RNA-guided DNA endonuclease in adaptive bacterial immunity”, Science 2012, 337:816-821), the PAM sequence recognized by Cas9 of Staphylococcus aureus is “NNGRRT” or “NNGRR(N)”, the PAM sequence recognized by Neisseria meningitidis Cas9 is NNNNGATT, and the PAM sequence recognized by Streptococcus thermophilus Cas9 is NNAGAAW. In this way, the editable window of a DNA molecule is larger.

In a specific embodiment, the targeted nuclease is any CRISPR/Cas nuclease capable of achieving genome editing.

In a specific embodiment, the targeted nuclease is in a form of DNA.

In another specific embodiment, the targeted nuclease is in a form of mRNA or protein instead of DNA. The protein form is preferred.

In a specific embodiment, the method for delivering targeted nucleases into a cell is selected from, but not limited to: 1) a PEG-mediated cell transfection method; 2) a liposome-mediated cell transfection method; 3) an electroporation transformation method; 4) a microinjection; 5) a gene gun bombardment; 6) an Agrobacterium-mediated transformation method.

In the method, a new target is designed based on a new sequence generated from the previous DNA break repair, and thus, mutations can be sequentially formed for many times at a specific site in the genome, thereby exponentially enriching types of repair events after DNA breaks, and generating new types of base substitution, deletion and insertion mutations that cannot be obtained by a single gene editing, so that the method is suitable to be used as a tool to create new mutations. This method can be briefly described as a method of programmed sequential cutting/editing or successive cutting/editing.

In a specific embodiment, a new target is designed based on a new specific sequence predicted to be generated from previous break repair at a specific site of organism genome, sequential editing is then performed, and thus a final possible mutation at the site can be designed in advance to achieve an expected editing.

In another specific embodiment, a new target is designed based on a new sequence predicted to be generated from previous break repair at a specific site of organism genome, sequential editing is then performed, and in addition to an expected editing event, a variety of different mutations can be generated eventually at the site, so that the method can be used as a tool to create various different mutations.

In another aspect, the present invention further provides a method for generating a new mutation in an organism, which comprises the following step: sequentially generating two or more DNA breaks at a specific site in a gene at the level of genome or chromosome of the organism, thereby achieving a precise base substitution, deletion or insertion.

In a specific embodiment, the “sequentially generating two or more DNA breaks at a specific site” refers to that a new target RNA designed based on a new sequence generated from a previous break repair event, and a cutting is performed again at the same site.

In a specific embodiment, the “DNA break” is achieved by a nuclease with targeting property.

The present invention further provides a new mutation obtained by the aforementioned method.

The present invention further provides a protein or biologically active fragment thereof that has the aforementioned new mutation.

The present invention further provides a nucleic acid, which comprises a nucleic acid sequence or complementary sequence thereof that encodes the protein or biologically active fragment thereof.

The present invention further provides a nucleic acid, which comprises:
(a) a nucleotide sequence encoding a target RNA, wherein
the target RNA comprises at least two target RNAs, in which a first target RNA targets a DNA to cause a break in the DNA, and a latter target RNA targets a sequence generated from a previous break repair event and generates a break again.

In a specific embodiment, the nucleic acid further comprises (b) a nucleotide sequence encoding a Cas polypeptide.

In a specific embodiment, the target RNA is a sgRNA or gRNA.

In a specific embodiment, the Cas polypeptide and the target RNA are present in an in vitro cell or an ex vivo cell.

The present invention further provides a recombinant expression vector, which comprises the aforementioned nucleic acid and a promoter operably linked thereto.

The present invention further provides an expression cassette, which comprises the aforementioned nucleic acid.

The present invention further provides a host cell, which comprises the aforementioned expression cassette.

The present invention further provides an organism that is regenerated by using the aforementioned host cell.

The present invention further provides a method for lysing a target DNA, which comprises contacting the target DNA with a complex, wherein the complex comprises:
(a) a Cas polypeptide; and
(b) at least two target RNAs, in which a first targets RNA targets the DNA to cause a break in the DNA, and a latter target RNA targets a sequence generated from a previous break repair event and generates a break again.

In a specific embodiment, the target RNA is a sgRNA or gRNA.

In a specific embodiment, the target DNA is present in a bacterial cell, eukaryotic cell, plant cell or animal cell.

In a specific embodiment, the target DNA is a chromosomal DNA.

In a specific embodiment, the Cas polypeptide and the target RNA are present in an in vitro cell or an ex vivo cell.

In a specific embodiment, the contacting comprises introducing the following into a cell: (a) the Cas polypeptide or a polynucleotide encoding the Cas polypeptide, and (b) the target RNA or a DNA polynucleotide encoding the target RNA.

The present invention further provides a composition, which comprises:
(a) a Cas polypeptide, or a polynucleotide encoding the Cas polypeptide; and
(b) at least two target RNAs, or DNA polynucleotides encoding the target RNAs, wherein a first RNA targets a DNA to cause a break in the DNA, and a latter target RNA targets a sequence generated from a previous break repair event and generates a break again.

In a specific embodiment, the target RNA is a sgRNA or gRNA.

In a specific embodiment, the Cas polypeptide and the target RNA are present in an in vitro cell or an ex vivo cell.

The invention further provides a use of the composition in manufacture of a medicament for treatment of a disease.

The disease that can be treated with the composition of the present invention includes, but is not limited to, a disease caused by single gene mutation, such as genetic tyrosinemia type 1, phenylketonuria, progeria, sickle cell disease, etc. A spontaneous cell repair is induced by delivering into a cell the Cas protein and the crRNA or sgRNA composition that is expected to repair a pathogenic mutation site to produce a normal functional protein, and thus a therapeutic effect is obtained.

The present invention further provides a kit, which comprises:
(a) a Cas polypeptide, or a nucleic acid comprising a nucleotide sequence encoding the Cas polypeptide; and
(b) at least two target RNAs, or nucleic acids comprising nucleotide sequences encoding the target RNAs, wherein a first target RNA targets a DNA to cause a break in the DNA, and a latter target RNA targets a sequence generated from a previous break repair event and generates a break again;
wherein (a) and (b) are in a same or separate containers.

In a specific embodiment, the target RNA is a sgRNA or gRNA.

In a specific embodiment, the target RNAs in (b) are in a same or separate containers.

The present invention further provides a method for screening editing events independent of exogenous transgenic markers, comprising the following steps:
1) two or more DNA breaks are sequentially generated in sequence at a specific site of a first target gene of a recipient cell and spontaneously repaired respectively, wherein a later DNA break is generated based on a new sequence generated from a repair of a previous DNA break;
2) certain editing events are generated after the specific site of the first target gene is sequentially cut and repaired, which can confer a mutant cell with a resistance to a certain selection pressure to produce a phenotypic selectable trait, a corresponding selection pressure is applied to make a selection for the trait, and a cell, tissue, organ or complete organism that contains such editing events is isolated;
3) optionally, in addition to the first target gene, a targeted nuclease for at least one second target gene is used to edit another target site at the same time, and an editing event of the second target gene is enriched and screened synchronously through the screening of the selectable trait generated by mutations of the first target gene, and a cell, tissue, organ or complete organism that simultaneously contains the editing events of the first target gene and of the at least one second target gene is isolated.

In a specific embodiment, the “first target gene” is a gene locus encoding at least one phenotypic selectable trait, wherein the at least one phenotypic selectable trait is a resistance/tolerance trait or a growth advantage trait.

In a specific embodiment, the “specific site of a first target gene” refers to a site at which a certain type of mutation is generated after sequential cuttings and repairs, which is capable of conferring the recipient cell with a resistance to a certain selection pressure to produce at least one phenotypic selectable resistance/tolerance trait or growth advantage trait.

In a specific embodiment, the “certain type of mutation” comprises substitution of single base, substitution of a plurality of bases, or insertion or deletion of an unspecified number of bases.

In a specific embodiment, the “certain selection pressure” may be an environmental pressure or a pressure resulted from an added compound; for example, the environmental pressure is high temperature, low temperature or hypoxia and the like; the pressure resulted from an added compound may be a pressure resulted from a salt ion concentration, antibiotic, cytotoxin, herbicide, etc.

In a specific embodiment, the “DNA break” is achieved by delivering a nuclease with a targeting property into a cell of an organism to contact with a specific site of genomic DNA.

In a specific embodiment, the “nuclease with a targeting property” is any CRISPR/Cas nuclease capable of performing genome editing.

In a specific embodiment, the feature, “two or more DNA breaks are sequentially generated in sequence at a specific site”, refers to that based on a new sequence formed by a previous DNA break repair event generated by a CRISPR/Cas system, a new target RNA is designed to cut the site again.

In a specific embodiment, the “two or more DNA breaks” are generated when RNP complexes formed by a same CRISPR/Cas nuclease respectively with different gRNAs or sgRNAs sequentially cut corresponding target sequences.

In another specific embodiment, the “two or more DNA breaks” are generated when RNP complexes respectively formed by each of two or more CRISPR/Cas nucleases that recognize different PAM sequences with respective gRNA or sgRNA, sequentially cut corresponding target sequences. In this way, the editable window of a DNA molecule is larger.

In a specific embodiment, the “second target gene” refers to another gene that is different in coding from the first target gene.

In a specific embodiment, the “targeted nuclease for at least one second target gene” and the CRISPR/Cas nuclease used for generating DNA break at a specific site of the first target gene are the same.

In another specific embodiment, the “targeted nuclease for at least one second target gene” and the CRISPR/Cas nuclease used for generating DNA break at a specific site of the first target gene are different. In this way, there are more selectable editing sites on the second target gene.

In a specific embodiment, the targeted nuclease is in a form of DNA.

In another specific embodiment, the targeted nuclease is in a form of mRNA or protein instead of DNA. The protein form is preferred.

In a specific embodiment, the method for delivering targeted nuclease into cell is selected from, but not limited to: 1) a PEG-mediated cell transfection method; 2) a liposome-mediated cell transfection method; 3) an electroporation transformation method; 4) a microinjection; 5) a gene gun bombardment; or 6) an Agrobacterium-mediated transformation method.

The present invention further provides a method for non-transgenic transient editing of an organism genome, comprising the following steps:
1) a combination of at least two crRNA fragments or a combination of at least two sgRNA fragments is designed and synthesized for a specific site of a first target gene of a recipient cell, wherein the crRNA combination combined with tracrRNA or the sgRNA combination alone is capable of guiding a corresponding Cas protein to sequentially generate two or more DNA breaks at the specific site in the first target gene of the recipient cell and to spontaneously repair them respectively, wherein a later DNA break is generated based on a new sequence generated from a previous DNA break repair;
2) an appropriate amount of CRISPR/Cas protein or corresponding mRNA thereof is mixed with the combination of crRNA fragments and tracrRNA fragment or with the combination of sgRNA fragments alone as above designed and synthesized in advance that is capable of guiding a site-specific editing of the first target gene to generate endogenous selection markers, optionally, at least one of artificially synthesized crRNA and tracrRNA fragments or artificially synthesized sgRNA fragments targeting a second, third or more target genes is further added, and incubation is carried out in vitro to form an RNP complex;
3) the above RNP complex is delivered into the recipient cell and contacts with the specific site of genomic DNA to achieve gene editing;
4) according to a phenotypic selectable trait generated by a site-specific editing of the first target gene by the RNP complex, a corresponding selection pressure is applied to make a selection for the trait, and a cell, tissue, organ or complete organism that contains the editing event is isolated, and optionally, a cell, tissue, organ or complete organism that simultaneously contains the editing events of the first target gene and of the at least one of a second, third or more target genes is isolated.

In a specific embodiment, the “first target gene” is a gene locus encoding at least one phenotypic selectable trait, wherein the at least one phenotypic selectable trait is a resistance/tolerance trait or a growth advantage trait.

In a specific embodiment, the “specific site of the first target gene” refers to a site at which a certain type of mutation is generated after sequential cuttings and repairs, which is capable of conferring the recipient cell with a resistance to a certain selection pressure to produce at least one phenotypic selectable resistance/tolerance trait or growth advantage trait.

In a specific embodiment, the “certain type of mutation” comprises substitution of single base, substitution of a plurality of bases, or insertion or deletion of an unspecified number of bases.

In a specific embodiment, the “certain selection pressure” may be an environmental pressure or a pressure resulted from an added compound; for example, the environmental pressure is high temperature, low temperature or hypoxia and the like; the pressure resulted from an added compound may be a pressure resulted from a salt ion concentration, antibiotic, cytotoxin or herbicide, and the like.

In a specific embodiment, the CRISPR/Cas protein is any CRISPR/Cas nuclease capable of performing genome editing.

In a specific embodiment, the feature “to sequentially generate two or more DNA breaks at the specific site” refers to that based on a new sequence formed by a previous DNA break repair event generated by a CRISPR/Cas system, a new target RNA is designed to cut the site again.

In a specific embodiment, the “two or more DNA breaks” are generated when RNP complexes formed by a same CRISPR/Cas nuclease respectively with different gRNAs or sgRNAs sequentially cut corresponding target sequences.

In another specific embodiment, the “two or more DNA breaks” are generated when RNP complexes respectively formed by each of two or more CRISPR/Cas nucleases that recognize different PAM sequences with respective gRNA or sgRNA, sequentially cut corresponding target sequences. In this way, the editable window of a DNA molecule is larger.

In a specific embodiment, the “second, third or more target genes” refer to other genes that are different in coding from the first target gene.

In a specific embodiment, the “at least one of artificially synthesized crRNA and tracrRNA fragments or artificially synthesized sgRNA fragments targeting a second, third or more target genes” shares the same Cas protein with the crRNA or sgRNA targeting the first target gene.

In another specific embodiment, the “at least one of artificially synthesized crRNA and tracrRNA fragments or artificially synthesized sgRNA fragments targeting a second, third or more target genes” and the crRNA or sgRNA targeting the first target gene use Cas proteins that recognize different PAM sequences. In this way, there are more selectable editing sites on the second target gene.

In a specific embodiment, the method for delivering the RNP complex into cells is selected from, but not limited to: 1) a PEG-mediated cell transfection method; 2) a liposome-mediated cell transfection method; 3) an electroporation transformation method; 4) a microinjection; 5) a gene gun bombardment; and so on.

The present invention further provides a method for non-transgenic transient editing of a plant genome, comprising the following steps:
1) a combination of at least two crRNA fragments or a combination of at least two sgRNA fragments is designed and synthesized for a specific site of a first target gene of a recipient plant cell or tissue, the crRNA combination combined with tracrRNA or the sgRNA combination alone is capable of guiding a corresponding Cas protein to sequentially generate two or more DNA breaks at the specific site in the first target gene of the recipient cell and to spontaneously repair them respectively, wherein a later DNA break is generated based on a new sequence generated from a previous DNA break repair;
2) an appropriate amount of CRISPR/Cas protein or corresponding mRNA thereof is mixed with the combination of crRNA fragments and tracrRNA fragment or with the combination of sgRNA fragments alone as above designed and synthesized in advance that is capable of guiding the site-specific editing of the first target gene to generate endogenous selection markers, optionally, at least one of artificially synthesized crRNA and tracrRNA fragments or artificially synthesized sgRNA fragments targeting a second, third or more target genes is further added, and incubation is carried out in vitro to form an RNP complex;
3) the above RNP complex is delivered into the recipient plant cell or tissue and contacts with the specific site of genomic DNA to achieve gene editing;
4) according to a phenotypic selectable trait generated by a site-specific editing of the first target gene by the RNP complex, a corresponding selection pressure is applied to make a selection for the trait, and a cell, tissue, organ or complete plant that contains the editing event is isolated, and optionally, a cell, tissue, organ or complete plant that simultaneously contains the editing events of the first target gene and of the at least one of second, third or more target genes is isolated.

In a specific embodiment, the “first target gene” is a gene locus encoding at least one phenotypic selectable trait, wherein the at least one phenotypic selectable trait is a resistance/tolerance trait or a growth advantage trait.

In a specific embodiment, the “specific site of the first target gene” refers to a site at which a certain type of mutation is generated after sequential cuttings and repairs at the site, which can confer the recipient cell with a resistance to a certain selection pressure to produce at least one phenotypic selectable resistance/tolerance trait or growth advantage trait.

In a specific embodiment, the “certain type of mutation” comprises substitution of single base, substitution of a plurality of bases, or insertion or deletion of an unspecified number of bases.

In a specific embodiment, the “certain selection pressure” may be an environmental pressure or a pressure resulted from an added compound; for example, the environmental pressure is preferably high temperature, low temperature or hypoxia and the like; the pressure resulted from an added compound may be a pressure resulted from a salt ion concentration, antibiotic, cytotoxin, herbicide, etc.

In a specific embodiment, the “recipient plant cell or tissue” is any cell or tissue that can serve as a recipient for transient expression and can be regenerated into a complete plant through tissue culture. Specifically, the cell is a protoplast cell or a suspension cell; the tissue is preferably a callus, immature embryo, mature embryo, leaf, shoot tip, young spike, hypocotyl, etc.

In a specific embodiment, the CRISPR/Cas protein is any CRISPR/Cas nuclease capable of performing genome editing.

In a specific embodiment, the feature “to sequentially generate two or more DNA breaks at the specific site” refers to that based on a new sequence formed by a previous DNA break repair event generated by a CRISPR/Cas system, a new target RNA is designed to cut the site again.

In a specific embodiment, the “two or more DNA breaks” are generated when RNP complexes formed by a same CRISPR/Cas nuclease respectively with different gRNAs or sgRNAs sequentially cut corresponding target sequences.

In another specific embodiment, the “two or more DNA breaks” are generated when RNP complexes respectively formed by each of two or more CRISPR/Cas nucleases that recognize different PAM sequences with respective gRNA or sgRNA, sequentially cut corresponding target sequences. In this way, the editable window of a DNA molecule is larger.

In a specific embodiment, the “second, third or more target genes” refer to other genes that are different in coding from the first target gene.

In a specific embodiment, the “at least one of artificially synthesized crRNA and tracrRNA fragments or artificially synthesized sgRNA fragments targeting a second, third or more target genes” shares the same Cas protein with the crRNA or sgRNA targeting the first target gene.

In another specific embodiment, the “at least one of artificially synthesized crRNA and tracrRNA fragments or artificially synthesized sgRNA fragments targeting a second, third or more target genes” and the crRNA or sgRNA targeting the first target gene use Cas proteins that recognize different PAM sequences. In this way, there are more selectable editing sites on the second target gene.

In a specific embodiment, the method for delivering the RNP complex into plant cells is selected from, but not limited to: 1) a PEG-mediated protoplast transformation method; 2) a microinjection; 3) a gene gun bombardment; 4) a silicon carbide fiber-mediated method; 5) a vacuum infiltration method, or any other transient introduction method. The gene gun bombardment is preferred.

In a specific embodiment, the “first target gene” is at least one endogenous gene that encodes at least one phenotypic selectable trait selected from herbicide resistance/tolerance, wherein the herbicide resistance/tolerance is selected from the group consisting of resistance/tolerance to EPSPS inhibitor (including glyphosate); resistance/tolerance to glutamine synthesis inhibitor (including glufosinate); resistance/tolerance to ALS or AHAS inhibitor (including imidazoline or sulfonylurea); resistance/tolerance to ACCase inhibitor (including aryloxyphenoxypropionic acid (FOP)); resistance/tolerance to carotenoid biosynthesis inhibitor, including carotenoid biosynthesis inhibitors of phytoene desaturase (PDS) step, 4-hydroxyphenylpyruvate dioxygenase (HPPD) inhibitors or other carotenoid biosynthesis target inhibitors; resistance/tolerance to cellulose inhibitor; resistance/tolerance to lipid synthesis inhibitor; resistance/tolerance to long-chain fatty acid inhibitor; resistance/tolerance to microtubule assembly inhibitor; resistance/tolerance to photosystem I electron shunting agent; resistance/tolerance to photosystem II inhibitor (including carbamates, triazines and triazones); resistance/tolerance to PPO inhibitor; and resistance/tolerance to synthetic growth hormone (including dicamba, 2,4-D (i.e., 2,4-dichlorophenoxyacetic acid)). Wherein, the first target gene is selected from PsbA, ALS, EPSPS, ACCase, PPO, HPPD, PDS, GS, DOXPS, TIR1, AFB5, and some types of mutations generated after sequential cuttings and repairs at specific sites of these herbicide target genes may confer the recipient plant cells with resistance/tolerance to the corresponding herbicides.

In a specific embodiment, the “first target gene” is ALS, and the “specific site of gene” refers to site A122, P197, R198, D204, A205, D376, R377, W574, 5653 or G654 in an Arabidopsis AtALS protein amino acid sequence (e.g., as shown in SEQ ID NO:1), and amino acid sites in an ALS protein of another plant which correspond to the above-mentioned amino acid sites by using the AtALS amino acid sequence as reference standard. The crRNA or sgRNA targets a target sequence comprising a sequence encoding an AtALS protein amino acid sequence site selected from the group consisting of A122, P197, R198, D204, A205, D376, R377, W574, S653, G654 or any combination thereof, and a target sequence comprising a sequence encoding an amino acid site in an ALS protein of another plant which corresponds to the above-mentioned amino acid sites, and any combination thereof, by using the AtALS amino acid sequence as reference standard. The ALS W574 site is preferred. The selection pressure is preferably a treatment with pyroxsulam or nicosulfuron.

In a specific embodiment, the “first target gene” is ACCase, and the “specific site of gene” refers to site I1781, E1874, N1878, W1999, W2027, I2041, D2078, C2088 or G2096 in an Alopecurus myosuroides AmACCase protein amino acid sequence (e.g., as shown in SEQ ID NO: 3, and the gene sequence is as shown in SEQ ID NO: 4), and amino acid sites in an ACCase protein of another monocotyledonous plant which correspond to the above-mentioned amino acid sites by using the AmACCase amino acid sequence as reference standard. The crRNA or sgRNA targets a target sequence comprising a sequence encoding an AmACCase amino acid sequence site selected from the group consisting of I1781, E1874, N1878, W1999, W2027, I2041, D2078, C2088, G2096 or any combination thereof, and a target sequence comprising a sequence encoding an amino acid site in an ACCase protein of another monocotyledonous plant which corresponds to the above-mentioned amino acid site, and any combination thereof, by using the AmACCase amino acid sequence as reference standard. ACCase W2027 site is preferred. The selection pressure is preferably a treatment with quizalofop-p-ethyl.

In a specific embodiment, the “first target gene” is HPPD, and the “specific site of gene” refers to site H141, L276, P277, N338, G342, R346, D370, P386, K418 or G419 in an Oryza sativa OsHPPD protein amino acid sequence (as shown in SEQ ID NO: 5, and the genome sequence is as shown in SEQ ID NO: 6), and amino acid sites in an HPPD protein of another plant which correspond to the above-mentioned amino acid sites by using the OsHPPD amino acid sequence as reference standard. The crRNA or sgRNA targets a target sequence comprising a sequence encoding an OsHPPD amino acid sequence site selected from the group consisting of H141, L276, P277, N338, G342, R346, D370, P386, K418, G419 or any combination thereof, and a target sequence comprising a sequence encoding an amino acid site in an HPPD protein of another plant which corresponds to the above-mentioned amino acid site, and any combination thereof, by using the OsHPPD amino acid sequence as reference standard. The selection pressure is preferably a treatment with biscarfentrazone.

In a specific embodiment, the “first target gene” is PPO, and the “specific site of gene” refers to site S128, V217, S223, V364, K373, L423, Y425 or W470 in an Oryza sativa OsPPO1 protein amino acid sequence (as shown in SEQ ID NO: 7, and the genome sequence is as shown in SEQ ID NO: 8), and amino acid sites in a PPO protein of another plant which correspond to the above-mentioned amino acid sites by using the amino acid sequence of OsPPO1 as reference standard. The crRNA or sgRNA targets a target sequence comprising a sequence encoding an OsPPO1 amino acid sequence site selected from the group consisting of S128, V217, S223, V364, K373, L423, Y425, W470 or any combination thereof, and a target sequence comprising a sequence of the above-mentioned amino acid sites corresponding to a PPO protein of another plant and any combination thereof using the OsPPO1 amino acid sequence as reference standard. The selection pressure is preferably a treatment with saflufenacil.

In a specific embodiment, the “first target gene” is TIR1, and the “specific site of gene” refers to site F93, F357, C413 or S448 in an Oryza sativa OsTIR1 protein amino acid sequence (as shown in SEQ ID NO: 9, and the genome sequence is as shown in SEQ ID NO: 10), and amino acid sites in a TIR1 protein of another plant which correspond to the above-mentioned amino acid sites by using the OsTIR1 amino acid sequence as reference standard. The crRNA or sgRNA targets a target sequence comprising a sequence encoding an OsTIR1 amino acid sequence site selected from the group consisting of F93, F357, C413, S448 or any combination thereof, and a target sequence comprising a sequence encoding an amino acid site in a TIR1 protein of another plant which corresponds to the above-mentioned amino acid site, and any combination thereof, by using the OsTIR1 amino acid sequence as reference standard. The selection pressure is preferably 2,4-D treatment.

The present invention further provides a non-transgenic transient editing system using the aforementioned method.

The present invention additionally provides a use of the aforementioned non-transgenic transient editing system as a selection marker.

The present invention additionally provides a use of the aforementioned non-transgenic transient editing system in treatment of a disease.

The present invention additionally provides a use of the aforementioned non-transgenic transient editing system in biological breeding.

The present invention additionally provides a genetically modified plant obtained by the aforementioned method, the genome of which contains an editing event of a first target gene, and the genetically modified plant is obtained in a non-transgenic manner.

The present invention additionally provides a genetically modified plant obtained by the aforementioned method, the genome of which contains an editing event of a first target gene, and further contains at least one second target gene editing event, and the genetically modified plant is obtained in a non-transgenic manner.

The present invention additionally provides a genetically modified plant obtained by the aforementioned method, the genome of which contains at least one second target gene editing event, and the genetically modified plant is obtained in a non-transgenic manner, wherein the first target gene editing event has been removed by genetic separation.

The present invention further provides a genome of the genetically modified plant obtained by the aforementioned method, the genome comprising: 1) an editing event of a first target gene; 2) an editing event of the first target gene and an editing event of at least one second target gene; or 3) at least one second target gene editing event, wherein the editing event of the first target gene has been removed by genetic separation; wherein the genetically modified plant is obtained in a non-transgenic manner.

Another aspect of the present invention provides a new plant gene mutation obtained by the aforementioned method.

The present invention also provides a new mutation generated in a plant, which comprises one or a combination of two or more of the following types:
substitution of aspartic acid at a site corresponding to Arabidopsis ALS376 with any other amino acid, substitution of tryptophan at a site corresponding to Arabidopsis ALS574 with any other amino acid, substitution of serine at a site corresponding to Arabidopsis ALS653 with any other amino acid, or substitution of serine at a site corresponding to Arabidopsis ALS654 with any other amino acid; or substitution of tryptophan at a site corresponding to Alopecurus myosuroides ACCase2027 with any other amino acid.

In a specific embodiment, wherein the aspartic acid at a site corresponding to Arabidopsis ALS376 is substituted by glutamic acid (D376E), the tryptophan at a site corresponding to Arabidopsis ALS574 is substituted by leucine or methionine (W574L or W574M), the serine at a site corresponding to Arabidopsis ALS653 is substituted by asparagine or arginine (S653N or S653R), or the glycine at a site corresponding to Arabidopsis ALS654 is substituted by aspartic acid (G654D), wherein the sites of amino acids are mentioned by using the sites of corresponding amino acids in Arabidopsis thalianan as reference; or, the tryptophan at a site corresponding to Alopecurus myosuroides ACCase2027 is substituted by leucine or cysteine (W2027L or W2027C), wherein the site of amino acid is mentioned by using the site of corresponding amino acid in Alopecurus myosuroides as reference.

In another specific embodiment, the mutation type is S653R/G654D, wherein the sites of amino acids are mentioned by using the sites of corresponding amino acids in Arabidopsis thalianan as reference.

In a specific embodiment, the aspartic acid at site 350 of Oryza sativa ALS is substituted by any other amino acid, the tryptophan at site 548 of Oryza sativa ALS is substituted by any other amino acid, or the tryptophan at site 561 of Solanum tuberosum L. ALS2 is substituted by any other amino acid; or, the tryptophan at site 2038 of Oryza sativa ACCase2 is substituted by any other amino acid.

In another specific embodiment, the aspartic acid at site 350 of Oryza sativa ALS is substituted by glutamic acid (D350E), the tryptophan at site 548 of Oryza sativa ALS is substituted by leucine or methionine (W548L or W548M), or the tryptophan at site 561 of Solanum tuberosum L. ALS2 is substituted by leucine or methionine (W561L or W561M); or, the tryptophan at site 2038 of Oryza sativa ACCase2 is substituted by leucine or cysteine (W2038L or W2038C), wherein the amino acid sequence of the Oryza sativa ALS protein is shown in SEQ ID NO: 11, the amino acid sequence of the Solanum tuberosum L. StALS2 protein is shown in SEQ ID NO: 19, and the amino acid sequence of the Oryza sativa ACCase2 protein is shown in SEQ ID NO: 13.

The present invention additionally provides a protein or biologically active fragment thereof that has the aforementioned new mutation.

The present invention also provides a nucleic acid, which comprises a nucleic acid sequence or complementary sequence thereof that encodes the protein or biologically active fragment thereof.

The present invention additionally provides a recombinant expression vector, which comprises the nucleic acid and a promoter operably linked thereto.

The present invention further provides an expression cassette, which comprises the nucleic acid.

The present invention further provides a plant cell, which comprises the expression cassette.

The present invention further provides a plant regenerated by using the plant cell.

Another aspect of the present invention provides a method for producing a plant with improved resistance or tolerance to herbicides, which comprises regenerating the plant cell into a plant.

Another aspect of the present invention provides a method for controlling weeds in a plant cultivation site, wherein the plant includes the aforementioned plant or a plant produced by the aforementioned method, wherein the method comprises applying to the cultivation site one or more herbicides in an effective amount to control the weeds.

Another aspect of the present invention also provides a use of the new mutation, the protein or biologically active fragment thereof, the nucleic acid, the recombinant expression vector or the expression cassette in improving resistance or tolerance of a plant cell, a plant tissue, a plant part or a plant to herbicides.

The present invention has the following excellent technical effects:

Based on sequences generated from new repair event generated by sequential editing, new targets can be designed, which can sequentially form mutations for many times at a specific site in the genome, thereby exponentially enriching the types of repair events after DNA breaks, and realizing new types of base substitution, deletion and insertion mutations that cannot be obtained by a single gene editing. That is, the programmed sequential cutting/editing scheme adopted by the present invention, which uses the sequence generated from a previous gene editing repair as the later gene editing target, can endow CRISPR/Cas with new functions of single-base editing and site-precise deletion and insertion through simple knockout.

The invention can realize the screening of gene editing events in the absence of exogenous markers, further realize the non-transgenic gene editing and can effectively screen editing events, and can greatly reduce the biological safety concerns of the method in cell therapy and biological breeding.

In particular, the plant non-transgenic transient editing method provided by the present invention only involves Cas protein and artificially synthesized small fragments of gRNA or sgRNA, without the participation of exogenous DNA in the whole process, and produces endogenous resistance selection markers by editing the first target gene via continuous targeting, so that the editing event can be effectively screened, actually without genetic modification operations involved, and thus the method is equivalent to chemical mutagenesis or radiation-induced breeding, also does not require continuous multiple generations of separation and detection of exogenous transgenic components, thereby shortening the breeding cycle, ensuring the biological safety, saving supervision and approval cost, and providing great application prospects in precise breeding of plants.

### DETAILED DESCRIPTION OF INVENTION

In the present invention, unless otherwise specified, the scientific and technical terms used herein have the meanings commonly understood by those skilled in the art. In addition, protein and nucleic acid chemistry, molecular biology, cell and tissue culture, microbiology, immunology related terms and laboratory procedures used herein are all terms and routine procedures widely used in the corresponding fields. At the same time, in order to better understand the present invention, definitions and explanations of related terms are provided below.

The term “genome” as used herein refers to all complements of genetic material (genes and non-coding sequences) present in each cell or virus or organelle of an organism, and/or complete genome inherited from a parent as a unit (haploid).

The term “gene editing” refers to strategies and techniques for targeted specific modification of any genetic information or genome of living organisms. Therefore, the term includes editing of gene coding regions, but also includes editing of regions other than gene coding regions of the genome. It also includes editing or modifying other genetic information of nuclei (if present) and cells.

The term “CRISPR/Cas nuclease” may be a CRISPR-based nuclease or a nucleic acid sequence encoding the same, including but not limited to: 1) Cas9, including SpCas9, ScCas9, SaCas9, xCas9, VRER-Cas9, EQR-Cas9, SpG-Cas9, SpRY-Cas9, SpCas9-NG, NG-Cas9, NGA-Cas9 (VQR), etc.; 2) Cas12, including LbCpf1, FnCpf1, AsCpf1, MAD7, etc., or any variant or derivative of the aforementioned CRISPR-based nuclease; preferably, wherein the at least one CRISPR-based nuclease comprises a mutation compared to the corresponding wild-type sequence, so that the obtained CRISPR-based nuclease recognizes a different PAM sequence. As used herein, “CRISPR-based nuclease” is any nuclease that has been identified in a naturally occurring CRISPR system, which is subsequently isolated from its natural background, and has preferably been modified or combined into a recombinant construct of interest, suitable as a tool for targeted genome engineering. As long as the original wild-type CRISPR-based nuclease provides DNA recognition, i.e., binding properties, any CRISPR-based nuclease can be used and optionally reprogrammed or otherwise mutated so as to be suitable for various embodiments of the invention.

The term “CRISPR” refers to a sequence-specific genetic manipulation technique that relies on clustered regularly interspaced short palindromic repeats, which is different from RNA interference that regulates gene expression at the transcriptional level.

“Cas9 nuclease” and “Cas9” are used interchangeably herein, and refer to RNA-guided nuclease comprising Cas9 protein or fragment thereof (for example, a protein containing the active DNA cleavage domain of Cas9 and/or the gRNA binding domain of Cas9). Cas9 is a component of the CRISPR/Cas (clustered regularly interspaced short palindrome repeats and associated systems) genome editing system. It can target and cut DNA target sequences under the guidance of guide RNA to form DNA double-strand breaks (DSB).

“Cas protein” or “Cas polypeptide” refers to a polypeptide encoded by Cas (CRISPR-associated) gene. Cas protein includes Cas endonuclease. Cas protein can be a bacterial or archaeal protein. For example, the types I to III CRISPR Cas proteins herein generally originate from prokaryotes; the type I and type III Cas proteins can be derived from bacteria or archaea species, and the type II Cas protein (i.e., Cas9) can be derived from bacterial species. “Cas proteins” include Cas9 protein, Cpf1 protein, C2c1 protein, C2c2 protein, C2c3 protein, Cas3, Cas3-HD, Cas5, Cas7, Cas8, Cas10, Cas12a, Cas12b, or a combination or complex thereof.

“Cas9 variant” or “Cas9 endonuclease variant” refers to a variant of the parent Cas9 endonuclease, wherein when associated with crRNA and tracRNA or with sgRNA, the Cas9 endonuclease variant retains the abilities of recognizing, binding to all or part of a DNA target sequence and optionally unwinding all or part of a DNA target sequence, nicking all or part of a DNA target sequence, or cutting all or part of a DNA target sequence. The Cas9 endonuclease variants include the Cas9 endonuclease variants described herein, wherein the Cas9 endonuclease variants are different from the parent Cas9 endonuclease in the following manner the Cas9 endonuclease variants (when complexed with gRNA to form a polynucleotide-directed endonuclease complex capable of modifying a target site) have at least one improved property, such as, but not limited to, increased transformation efficiency, increased DNA editing efficiency, decreased off-target cutting, or any combination thereof, as compared to the parent Cas9 endonuclease (complexed with the same gRNA to form a polynucleotide-guided endonuclease complex capable of modifying the same target site).

The Cas9 endonuclease variants described herein include variants that can bind to and nick double-stranded DNA target sites when associated with crRNA and tracrRNA or with sgRNA, while the parent Cas endonuclease can bind to the target site and result in double strand break (cleavage) when associated with crRNA and tracrRNA or with sgRNA.

“Guide RNA” and “gRNA” are used interchangeably herein, and refer to a guide RNA sequence used to target a specific gene for correction using CRISPR technology, which usually consists of crRNA and tracrRNA molecules that are partially complementary to form a complex, wherein crRNA contains a sequence that has sufficient complementarity with the target sequence so as to hybridize with the target sequence and direct the CRISPR complex (Cas9+crRNA+tracrRNA) to specifically bind to the target sequence. However, it is known in the art that a single guide RNA (sgRNA) can be designed, which contains both the properties of crRNA and tracrRNA.

The terms “single guide RNA” and “sgRNA” are used interchangeably herein, and refer to the synthetic fusion of two RNA molecules, which comprises a fusion of a crRNA (CRISPR RNA) of a variable targeting domain (linked to a tracr pairing sequence hybridized to tracrRNA) and a tracrRNA (trans-activating CRISPR RNA). The sgRNA may comprise crRNA or crRNA fragments and tracrRNA or tracrRNA fragments of the type II CRISPR/Cas system that can form a complex with the type II Cas endonuclease, wherein the guide RNA/Cas endonuclease complex can guide the Cas endonuclease to a DNA target site so that the Cas endonuclease can recognize, optionally bind to the DNA target site, and optionally nick the DNA target site or cut (introduce a single-strand or double-strand break) the DNA target site.

In certain embodiments, the guide RNA(s) and Cas9 can be delivered to a cell as a ribonucleoprotein (RNP) complex. RNP is composed of purified Cas9 protein complexed with gRNA, and it is well known in the art that RNP can be effectively delivered to many types of cells, including but not limited to stem cells and immune cells (Addgene, Cambridge, Mass., Mirus Bio LLC, Madison, Wis.).

The protospacer adjacent motif (PAM) herein refers to a short nucleotide sequence adjacent to a (targeted) target sequence (prespacer) recognized by the gRNA/Cas endonuclease system. If the target DNA sequence is not adjacent to an appropriate PAM sequence, the Cas endonuclease may not be able to successfully recognize the target DNA sequence. The sequence and length of PAM herein can be different depending on the Cas protein or Cas protein complex in use. The PAM sequence can be of any length, but is typically in length of 1, 2, 3, 4, 5, 6, 7, 8, 9, 10, 11, 12, 13, 14, 15, 16, 17, 18, 19 or 20 nucleotides.

As used herein, the term “organism” or “living body” includes animals, plants, fungi, bacteria, and the like.

As used herein, the term “host cell” includes plant cells, animal cells, fungal cells, bacterial cells, and the like.

In the present invention, “animal” includes but is not limited to vertebrates, such as humans, non-human mammals, birds, fish, reptiles, amphibians, etc., as well as invertebrates, such as insects.

In the present invention, the “plant” should be understood to mean any differentiated multicellular organism capable of performing photosynthesis, in particular monocotyledonous or dicotyledonous plants, for example, (1) food crops: Oryza spp., like Oryza sativa, Oryza latifolia, Oryza sativa, Oryza glaberrima; Triticum spp., like Triticum aestivum, T. Turgidumssp. durum; Hordeum spp., like Hordeum vulgare, Hordeum arizonicum; Secale cereale; Avena spp., like Avena sativa, Avena fatua, Avena byzantine, Avena fatua var. sativa, Avena hybrida; Echinochloa spp., like Pennisetum glaucum, Sorghum, Sorghum bicolor, Sorghum vulgare, Triticale, Zea mays or Maize, Millet, Rice, Foxtail millet, Proso millet, Sorghum bicolor, Panicum, Fagopyrum spp., Panicum miliaceum, Setaria italica, Zizania palustris, Eragrostis tef, Panicum miliaceum, Eleusine coracana; (2) legume crops: Glycine spp. like Glycine max, Soja hispida, Soja max, Vicia spp., Vigna spp., Pisum spp., field bean, Lupinus spp., Vicia, Tamarindus indica, Lens culinaris, Lathyrus spp., Lablab, broad bean, mung bean, red bean, chickpea; (3) oil crops: Arachis hypogaea, Arachis spp, Sesamum spp., Helianthus spp. like Helianthus annuus, Elaeis like Eiaeis guineensis, Elaeis oleifera, soybean, Brassicanapus, Brassica oleracea, Sesamum orientale, Brassica juncea, Oilseed rape, Camellia oleifera, oil palm, olive, castor-oil plant, Brassica napus L., canola; (4) fiber crops: Agave sisalana, Gossypium spp. like Gossypium, Gossypium barbadense, Gossypium hirsutum, Hibiscus cannabinus, Agave sisalana, Musa textilis Nee, Linum usitatissimum, Corchorus capsularis L, Boehmeria nivea (L.), Cannabis sativa, Cannabis sativa; (5) fruit crops: Ziziphus spp., Cucumis spp., Passiflora edulis, Vitis spp., Vaccinium spp., Pyrus communis, Prunus spp., Psidium spp., Punica granatum, Malus spp., Citrullus lanatus, Citrus spp., Ficus carica, Fortunella spp., Fragaria spp., Crataegus spp., Diospyros spp., Eugenia unifora, Eriobotrya japonica, Dimocarpus longan, Carica papaya, Cocos spp., Averrhoa carambola, Actinidia spp., Prunus amygdalus, Musa spp. (Musa acuminate), Persea spp. (Persea Americana), Psidium guajava, Mammea americana, Mangifera indica, Canarium album (Oleaeuropaea), Caricapapaya, Cocos nucifera, Malpighia emarginata, Manilkara zapota, Ananas comosus, Annona spp., Citrus reticulate (Citrus spp.), Artocarpus spp., Litchi chinensis, Ribes spp., Rubus spp., pear, peach, apricot, plum, red bayberry, lemon, kumquat, durian, orange, strawberry, blueberry, hami melon, muskmelon, date palm, walnut tree, cherry tree; (6) rhizome crops: Manihot spp., Ipomoea batatas, Colocasia esculenta, tuber mustard, Allium cepa (onion), eleocharis tuberose (water chestnut), Cyperus rotundus, Rhizoma dioscoreae; (7) vegetable crops: Spinacia spp., Phaseolus spp., Lactuca sativa, Momordica spp, Petroselinum crispum, Capsicum spp., Solanum spp. (such as Solanum tuberosum, Solanum integrifolium, Solanum lycopersicum), Lycopersicon spp. (such as Lycopersicon esculentum, Lycopersicon lycopersicum, Lycopersicon pyriforme), Macrotyloma spp., Kale, Luffa acutangula, lentil, okra, onion, potato, artichoke, asparagus, broccoli, Brussels sprouts, cabbage, carrot, cauliflower, celery, collard greens, squash, Benincasa hispida, Asparagus officinalis, Apium graveolens, Amaranthus spp., Allium spp., Abelmoschus spp., Cichorium endivia, Cucurbita spp., Coriandrum sativum, B. carinata, Rapbanus sativus, Brassica spp. (such as Brassica napus, Brassica rapa ssp., canola, oilseed rape, turnip rape, turnip rape, leaf mustard, cabbage, black mustard, canola (rapeseed), Brussels sprout, Solanaceae (eggplant), Capsicum annuum (sweet pepper), cucumber, luffa, Chinese cabbage, rape, cabbage, calabash, Chinese chives, lotus, lotus root, lettuce; (8) flower crops: Tropaeolum minus, Tropaeolum majus, Canna indica, Opuntia spp., Tagetes spp., Cymbidium (orchid), Crinum asiaticum L., Clivia, Hippeastrum rutilum, Rosa rugosa, Rosa Chinensis, Jasminum sambac, Tulipa gesneriana L., Cerasus sp., Pharbitis nil (L.) Choisy, Calendula officinalis L., Nelumbo sp., Bellis perennis L., Dianthus caryophyllus, Petunia hybrida, Tulipa gesneriana L., Lilium brownie, Prunus mume, Narcissus tazetta L., Jasminum nudiflorum Lindl., Primula malacoides, Daphne odora, Camellia japonica, Michelia alba, Magnolia liliiflora, Viburnum macrocephalum, Clivia miniata, Malus spectabilis, Paeonia suffruticosa, Paeonia lactiflora, Syzygium aromaticum, Rhododendron simsii, Rhododendron hybridum, Michelia figo (Lour.) Spreng., Cercis chinensis, Kerria japonica, Weigela florida, Fructus forsythiae, Jasminum mesnyi, Parochetus communis, Cyclamen persicum Mill., Phalaenophsis hybrid, Dendrobium nobile, Hyacinthus orientalis, Iris tectorum Maxim, Zantedeschia aethiopica, Calendula officinalis, Hippeastrum rutilum, Begonia semperflorenshybr, Fuchsia hybrida, Begonia maculataRaddi, Geranium, Epipremnum aureum; (9) medicinal crops: Carthamus tinctorius, Mentha spp., Rheum rhabarbarum, Crocus sativus, Lycium chinense, Polygonatum odoratum, Polygonatum Kingianum, Anemarrhena asphodeloides Bunge, Radix ophiopogonis, Fritillaria cirrhosa, Curcuma aromatica, Amomum villosum Lour., Polygonum multiflorum, Rheum officinale, Glycyrrhiza uralensis Fisch, Astragalus membranaceus, Panax ginseng, Panax notoginseng, Acanthopanax gracilistylus, Angelica sinensis, Ligusticum wallichii, Bupleurum sinenses DC., Datura stramonium Linn., Datura metel L., Mentha haplocalyx, Leonurus sibiricus L., Agastache rugosus, Scutellaria baicalensis, Prunella vulgaris L., Pyrethrum carneum, Ginkgo biloba L., Cinchona ledgeriana, Hevea brasiliensis (wild), Medicago sativa Linn, Piper Nigrum L., Radix Isatidis, Atractylodes macrocephala Koidz; (10) raw material crops: Hevea brasiliensis, Ricinus communis, Vernicia fordii, Morus alba L., Hops Humulus lupulus, Betula, Alnus cremastogyne Burk., Rhus verniciflua stokes; (11) pasture crops: Agropyron spp., Trifolium spp., Miscanthus sinensis, Pennisetum sp., Phalaris arundinacea, Panicum virgatum, prairiegrasses, Indiangrass, Big bluestem grass, Phleum pratense, turf, cyperaceae (Kobresia pygmaea, Carex pediformis, Carex humilis), Medicago sativa Linn, Phleum pratense L., Medicago sativa, Melilotus suavcolen, Astragalus sinicus, Crotalaria juncea, Sesbania cannabina, Azolla imbircata, Eichhornia crassipes, Amorpha fruticosa, Lupinus micranthus, Trifolium, Astragalus adsurgens pall, Pistia stratiotes linn, Alternanthera philoxeroides, Lolium; (12) sugar crops: Saccharum spp., Beta vulgaris; (13) beverage crops: Camellia sinensis, Camellia Sinensis, tea, Coffee (Coffea spp.), Theobroma cacao, Humulus lupulus Linn.; (14) lawn plants: Ammophila arenaria, Poa spp. (Poa pratensis (bluegrass)), Agrostis spp. (Agrostis matsumurae, Agrostis palustris), Lolium spp. (Lolium), Festuca spp. (Festuca ovina L.), Zoysia spp. (Zoysiajaponica), Cynodon spp. (Cynodon dactylon/bermudagrass), Stenotaphrum secunda turn (Stenotaphrum secundatum), Paspalum spp., Eremochloa ophiuroides (centipedegrass), Axonopus spp. (carpetweed), Bouteloua dactyloides (buffalograss), Bouteloua var. spp. (Bouteloua gracilis), Digitaria sanguinalis, Cyperusrotundus, Kyllingabrevifolia, Cyperusamuricus, Erigeron canadensis, Hydrocotylesibthorpioides, Kummerowiastriata, Euphorbia humifusa, Viola arvensis, Carex rigescens, Carex heterostachya, turf; (15) tree crops: Pinus spp., Salix spp., Acer spp., Hibiscus spp., Eucalyptus spp., Ginkgo biloba, Bambusa sp., Populus spp., Prosopis spp., Quercus spp., Phoenix spp., Fagus spp., Ceiba pentandra, Cinnamomum spp., Corchorus spp., Phragmites australis, Physalis spp., Desmodium spp., Populus, Hedera helix, Populus tomentosa Carr, Viburnum odoratissinum, Ginkgo biloba L., Quercus, Ailanthus altissima, Schima superba, Ilex pur-purea, Platanus acerifolia, Ligustrum lucidum, Buxus megistophylla Levl., Dahurian larch, Acacia mearnsii, Pinus massoniana, Pinus khasys, Pinus yunnanensis, Pinus finlaysoniana, Pinus tabuliformis, Pinus koraiensis, Juglans nigra, Citrus limon, Platanus acerifolia, Syzygium jambos, Davidia involucrate, Bombax malabarica L., Ceiba pentandra (L.), Bauhinia blakeana, Albizia saman, Albizzia julibrissin, Erythrina corallodendron, Erythrina indica, Magnolia gradiflora, Cycas revolute, Lagerstroemia indica, coniferous, macrophanerophytes, Frutex; (16) nut crops: Bertholletia excelsea, Castanea spp., Corylus spp., Carya spp., Juglans spp., Pistacia vera, Anacardium occidentale, Macadamia (Macadamia integrifolia), Carya illinoensis Koch, Macadamia, Pistachio, Badam, other plants that produce nuts; (17) others: Arabidopsis thaliana, Bra chiaria eruciformis, Cenchrus echinatus, Setaria faberi, Eleusine indica, Cadaba farinose, algae, Carex elata, ornamental plants, Carissa macrocarpa, Cynara spp., Daucus carota, Dioscorea spp., Erianthus sp., Festuca arundinacea, Hemerocallis fulva, Lotus spp., Luzula sylvatica, Medicago sativa, Melilotus spp., Morus nigra, Nicotiana spp., Olea spp., Ornithopus spp., Pastinaca sativa, Sambucus spp., Sinapis sp., Syzygium spp., Tripsacum dactyloides, Triticosecale rimpaui, Viola odorata, and the like.

In a specific embodiment, the plant is selected from rice, corn, wheat, soybean, sunflower, sorghum, rape, alfalfa, cotton, barley, millet, sugarcane, tomato, tobacco, cassava, potato, sweet potato, Chinese cabbage, cabbage, cucumber, Chinese rose, Scindapsus aureus, watermelon, melon, strawberry, blueberry, grape, apple, citrus, peach, pear, banana, etc.

As used herein, the term “plant” includes a whole plant and any progeny, cell, tissue or part of plant. The term “plant part” includes any part of a plant, including, for example, but not limited to: seed (including mature seed, immature embryo without seed coat, and immature seed); plant cutting; plant cell; plant cell culture; plant organ (e.g., pollen, embryo, flower, fruit, bud, leaf, root, stem, and related explant). Plant tissue or plant organ can be a seed, callus tissue, or any other plant cell population organized into a structural or functional unit. The plant cell or tissue culture can regenerate a plant that has the physiological and morphological characteristics of the plant from which the cell or tissue is derived, and can regenerate a plant that has substantially the same genotype as the plant. In contrast, some plant cells cannot regenerate plants. The regenerable cells in plant cells or tissue cultures can be embryos, protoplasts, meristematic cells, callus, pollen, leaves, anthers, roots, root tips, silk, flowers, kernels, spikes, cobs, husks, or stems.

The plant parts comprise harvestable parts and parts that can be used to propagate offspring plants. The plant parts that can be used for propagation include, for example, but not limited to: seeds; fruits; cuttings; seedlings; tubers; and rootstocks. The harvestable parts of plants can be any of useful parts of plants, including, for example, but not limited to: flowers; pollen; seedlings; tubers; leaves; stems; fruits; seeds; and roots.

The plant cells are the structural and physiological units of plants. As used herein, the plant cells include protoplasts and protoplasts with partial cell walls. The plant cells may be in a form of isolated single cells or cell aggregates (e.g., loose callus and cultured cells), and may be part of higher order tissue units (e.g., plant tissues, plant organs, and plants). Therefore, the plant cells can be protoplasts, gamete-producing cells, or cells or collection of cells capable of regenerating a whole plant. Therefore, in the embodiments herein, a seed containing a plurality of plant cells and capable of regenerating into a whole plant is considered as a “plant part”.

As used herein, the term “protoplast” refers to a plant cell whose cell wall is completely or partially removed and whose lipid bilayer membrane is exposed. Typically, the protoplast is an isolated plant cell without cell wall, which has the potential to regenerate a cell culture or a whole plant.

The plant “offspring” includes any subsequent generations of the plant.

The term “bacteria” means all prokaryotes, including all organisms in the Kingdom Procaryotae. The term “bacteria” includes all microorganisms considered to be bacteria, including Mycoplasma, Chlamydia, Actinomyces, Streptomyce, and Rickettsia. All forms of bacteria are included in this definition, including cocci, bacilli, spirilla, spheroplasts, protoplasts, etc. The term also includes prokaryotes that are Gram-negative or Gram-positive. “Gram-negative” and “Gram-positive”mean a staining pattern using Gram staining methods well known in the art (see, for example, Finegold and Martin, Diagnostic Microbiology, 6th Ed., CV Mosby St. Louis, pp. 13-15[1982]). “Gram-positive bacteria” are bacteria that can retain the original dye used for Gram staining, causing the stained cells to appear dark blue to purple under a microscope. “Gram-negative bacteria” do not retain the original dye used for Gram staining, but can be stained with a counter stain. Therefore, Gram-negative bacteria appear red after the Gram staining reaction.

As used herein, the term “fungi” refers to eukaryotic organisms such as molds and yeasts, including dimorphic fungi.

The terms “herbicide tolerance” and “herbicide resistance” can be used interchangeably, and both refer to herbicide tolerance and herbicide resistance. “Improvement in herbicide tolerance” and “improvement in herbicide resistance” mean that the tolerance or resistance to the herbicide is improved compared to a plant containing wild-type gene.

The term “wild-type” refers to a nucleic acid molecule or protein that can be found in nature.

In the present invention, the term “cultivation site” comprises a site where the plant of the present invention is cultivated, such as soil, and also comprises, for example, plant seeds, plant seedlings and grown plants. The term “weed-controlling effective amount” refers to an amount of herbicide that is sufficient to affect the growth or development of the target weed, for example, to prevent or inhibit the growth or development of the target weed, or to kill the weed. Advantageously, the weed-controlling effective amount does not significantly affect the growth and/or development of the plant seeds, plant seedlings or plants of the present invention. Those skilled in the art can determine such weed-controlling effective amount through routine experiments.

The term “Target DNA” as used herein refers to a DNA polynucleotide comprising a “target site” or “target sequence”.

The term “lysis” means the cleavage of the covalent backbone of a DNA molecule. The lysis can be initiated by a variety of methods, including but not limited to enzymatic or chemical hydrolysis of phosphodiester bonds. Both single-strand and double-strand lysis is possible, and double-strand lysis may occur due to two distinct single-strand lysis events. The DNA lysis may result in blunt or staggered ends. In certain embodiments, a complex comprising DNA-targeting RNA and a site-specific modification polypeptide is used for a targeted double-strand DNA lysis.

The term “gene” comprises a nucleic acid fragment expressing a functional molecule (such as, but not limited to, specific protein), including regulatory sequences before (5′ non-coding sequences) and after (3′ non-coding sequences) a coding sequence.

The DNA sequence that “encodes” a specific RNA is a DNA nucleic acid sequence that can be transcribed into RNA. The DNA polynucleotides can encode a RNA (mRNA) that can be translated into a protein, or the DNA polynucleotides can encode a RNA that cannot be translated into a protein (for example, tRNA, rRNA, or DNA-targeting RNA; which are also known as “non-coding” RNA or “ncRNA”).

The terms “polypeptide”, “peptide” and “protein” are used interchangeably in the present invention, and refer to a polymer of amino acid residues. The terms are applied to amino acid polymers in which one or more amino acid residues are artificially chemical analogs of corresponding and naturally occurring amino acids, as well as to naturally occurring amino acid polymers. The terms “polypeptide”, “peptide”, “amino acid sequence” and “protein” may also include their modification forms, including but not limited to glycosylation, lipid linkage, sulfation, γ-carboxylation of glutamic acid residue, hydroxylation and ADP-ribosylation.

The term “biologically active fragment” refers to a fragment that has one or more amino acid residues deleted from the N and/or C-terminus of a protein while still retaining its functional activity.

For the terms related to amino acid substitution used in the description, the first letter represents a naturally occurring amino acid at a certain position in a specific sequence, the following number represents the position in the corresponding sequence, and the second letter represents a different amino acid for substituting the naturally occurring amino acid. For example, W574L means that tryptophan at position 574 is substituted by leucine. For double or multiple mutations, each mutation is separated by “/”.

The terms “polynucleotide” and “nucleic acid” are used interchangeably and comprise DNA, RNA or hybrids thereof, which may be double-stranded or single-stranded.

The terms “nucleotide sequence” and “nucleic acid sequence” both refer to the sequence of bases in DNA or RNA.

As used in the present invention, “expression cassette”, “expression vector” and “expression construct” refer to a vector such as a recombinant vector suitable for expression of a nucleotide sequence of interest in a plant. The term “expression” refers to the production of a functional product. For example, the expression of a nucleotide sequence may refer to the transcription of the nucleotide sequence (such as transcription to generate mRNA or functional RNA) and/or the translation of RNA into a precursor or mature protein.

The “expression construct” of the present invention can be a linear nucleic acid fragment, a circular plasmid, a viral vector, or, in some embodiments, can be an RNA (such as mRNA) that can be translated.

The “expression construct” of the present invention may comprise regulatory sequences and nucleotide sequences of interest from different sources, or regulatory sequences and nucleotide sequences of interest from the same source but arranged in a way different from those normally occurring in nature.

The terms “recombinant expression vector” or “DNA construct” are used interchangeably herein and refer to a DNA molecule comprising a vector and at least one insert. Recombinant expression vectors are usually produced for the purpose of expression and/or propagation of the insert or for the construction of other recombinant nucleotide sequences. The insert may be operably or may be inoperably linked to a promoter sequence and may be operably or may be inoperably linked to a DNA regulatory sequence.

The terms “regulatory sequence” and “regulatory element” can be used interchangeably and refer to a nucleotide sequence that is located at the upstream (5′ non-coding sequence), middle or downstream (3′ non-coding sequence) of a coding sequence, and affects the transcription, RNA processing, stability or translation of a related coding sequence. Plant expression regulatory elements refer to nucleotide sequences that can control the transcription, RNA processing or stability or translation of a nucleotide sequence of interest in plants.

The regulatory sequences may include, but are not limited to, promoters, translation leader sequences, introns, and polyA recognition sequences.

The term “promoter” refers to a nucleic acid fragment capable of controlling the transcription of another nucleic acid fragment. In some embodiments of the present invention, the promoter is a promoter capable of controlling gene transcription in plant cells, regardless of whether it is derived from plant cells. The promoter can be a constitutive promoter or a tissue-specific promoter or a developmentally regulated promoter or an inducible promoter.

The term “constitutive promoter” refers to a promoter that will generally cause gene expression in most cell types in most cases. “Tissue-specific promoter” and “tissue-preferred promoter” are used interchangeably, and refer to a promoter that is mainly but not necessarily exclusively expressed in a tissue or organ, and also expressed in a specific cell or cell type. “Developmentally regulated promoter” refers to a promoter whose activity is determined by a developmental event. “Inducible promoter” responds to an endogenous or exogenous stimulus (environment, hormone, chemical signal, etc.) to selectively express an operably linked DNA sequence.

As used herein, the term “operably linked” refers to a connection of a regulatory element (for example, but not limited to, promoter sequence, transcription termination sequence, etc.) to a nucleic acid sequence (for example, a coding sequence or open reading frame) such that the transcription of the nucleotide sequence is controlled and regulated by the transcription regulatory element. The techniques for operably linking regulatory element region to nucleic acid molecule are known in the art.

The “introducing” a nucleic acid molecule (such as a plasmid, linear nucleic acid fragment, RNA, etc.) or protein into a plant refers to transforming a cell of the plant with the nucleic acid or protein so that the nucleic acid or protein can function in the plant cell. The term “transformation” used in the present invention comprises stable transformation and transient transformation.

The term “stable transformation” refers to that the introduction of an exogenous nucleotide sequence into a plant genome results in a stable inheritance of the exogenous gene. Once stably transformed, the exogenous nucleic acid sequence is stably integrated into the genome of the plant and any successive generations thereof.

The term “transient transformation” refers to that the introduction of a nucleic acid molecule or protein into a plant cell to perform function does not result in a stable inheritance of the foreign gene. In transient transformation, the exogenous nucleic acid sequence is not integrated into the genome of the plant.

Unless otherwise defined, all technical and scientific terms used herein have the same meanings as commonly understood by those of ordinary skill in the art to which the present invention pertains. Although any methods and materials similar or equivalent to those described herein can also be used in the practice or testing of the present invention, the preferred methods and materials are now described.

All publications and patents cited in this description are incorporated herein by reference as if each individual publication or patent is exactly and individually indicated to be incorporated by reference, and is incorporated herein by reference to disclose and describe methods and/or materials related to the publications cited. The citation of any publication which it was published before the filing date should not be interpreted as an admission that the present invention is not eligible to precede the publications of the existing invention. In addition, the publication date provided may be different from the actual publication date, which may require independent verification.

Unless specifically stated or implied, as used herein, the terms “a”, “a/an” and “the” mean “at least one.” All patents, patent applications, and publications mentioned or cited herein are incorporated herein by reference in their entirety, with the same degree of citation as if they were individually cited.

### BRIEF DESCRIPTION OF DRAWINGS

FIG. 1 shows a schematic diagram of the method for generating new mutations in organisms according to the present invention. In the figure, only Cas9 with NGG as PAM is taken as an example; in the same way, other Cas9 variants with different PAM (such as NG) can also be used.

FIG. 2 shows a schematic diagram of gRNA design at the ALS gene sites W574 and S653 of Arabidopsis thaliana.

FIG. 3 shows the Arabidopsis thaliana T2 generation herbicide-resistant strains transformed with two different programmed sequential cutting/editing vectors. pQY743 and pQY745 are vector numbers. Resistant strains were capable of rooting normally, but the wild-type Col-0 and non-resistant strains were not.

FIG. 4 shows a diagram of sequencing peaks of the ALS gene of the T2 generation strain of Arabidopsis thaliana resistant to imazapic, in which the T indicated by the arrow was mutated from G, resulting in a W574L mutation.

FIG. 5 shows a design of the programmed sequential cutting/editing scheme at W574 site of ALS gene of Arabidopsis thaliana. The T-DNA sequence expressed four genes, sgRNA1, sgRNA2, Cas9 and HygR. Among them, sgRNA1 and Cas9 formed a complex, which cut the W574 codon of ALS in the genome, and was expected to form −G genotype through cellular spontaneous repair. This new sequence could be recognized and cut by the complex formed by sgRNA2 and Cas9, and formed+T genotype through cellular spontaneous repair, leading to W574L.

FIG. 6 shows the resistant seedlings of Arabidopsis thaliana screened by imazapic and the sequencing results of ALS W574 site.

FIG. 7 shows a design of the programmed sequential cutting/editing scheme at S653 site of ALS gene of Arabidopsis thaliana. The T-DNA sequence expressed four genes, sgRNA1, sgRNA2, Cas9 and HygR. Among them, sgRNA1 and Cas9 formed a complex, which cut the S653 codon of ALS in the genome. After cellular spontaneous repair, a −G genotype was formed. This sequence could be recognized and cut by the complex formed by sgRNA2 and Cas9. After cellular spontaneous repair, a +A genotype was formed, resulting in S653N.

FIG. 8 shows the resistant seedlings of Arabidopsis thaliana screened by imazapic and the sequencing results of ALS 5653 site. The left panel shows the screening results of resistant seedlings and the ratio of resistant seedlings, and the right panel shows a diagram of sequencing peaks and mutation type of S653 site.

FIG. 9 shows a design of the programmed sequential cutting/editing scheme at W574 site of ALS gene of Arabidopsis thaliana. The T-DNA sequence expressed four genes, sgRNA1, sgRNA2, Cas9 and HygR. Among them, sgRNA1 and Cas9 formed a complex, which cut the W574 codon of ALS in the genome. After cellular spontaneous repair, the +A genotype was formed. This sequence could be recognized and cut by the complex formed by sgRNA2 and Cas9. After cellular spontaneous repair, the −G genotype was formed, resulting in W574M. The two cuttings used different PAM sites.

FIG. 10 shows a design of the programmed sequential cutting/editing scheme at W2038 site of ACCase2 gene of Oryza sativa. This site corresponds to site W2027 of ACCase2 gene of Alopecurus myosuroides. The T-DNA sequence expressed four genes, sgRNA1, sgRNA2, Cas9 and HygR. Among them, sgRNA1 and Cas9 formed a complex, which cut the W2038 codon of ACCase in the genome. A −G genotype was formed after cellular spontaneous repair. This sequence could be recognized and cut by the complex formed by sgRNA2 and Cas9. After cellular spontaneous repair, a +T genotype was formed, resulting in W2038L.

FIG. 11 shows the resistant callus of Oryza sativa co-screened with hygromycin (50 ug/L) and quizalofop-p (50 ug/L) and the sequencing results of W2038 site.

FIG. 12 shows a diagram of polyacrylamide gel electrophoresis of SpCas9 and NGA-Cas9 proteins which were produced by prokaryotic expression and purified. The band indicated by arrow is the Cas9 protein band.

FIG. 13 shows the in vitro cutting activity of purified Cas9 protein on DNA fragments containing OsALS W548 target site and OsACCase2 W2038 target site, which was detected by agarose gel electrophoresis, and it can be seen that only when Cas9 protein and sgRNA fragment were added at the same time, the DNA fragments could be cut to an expected size.

FIG. 14 shows a diagram of the sequencing peaks of the OsALS W548 target site of RNP transformed Oryza sativa protoplasts, and the site corresponds to ALS W574 site of Arabidopsis thaliana. At the place indicated by the arrow, in addition to the original G base signal peak, there was a T base signal peak obtained by mutation, which led to a W548L mutation.

FIG. 15 shows the resistant callus of Oryza sativa edited by RNP at OsACCase2 W2038 site and screened with 50 ug/L quizalofop-p. The arrow indicates the resistant callus.

FIG. 16 shows a diagram of sequencing peaks of the OsACCase2 W2038 target site of Oryza sativa seedlings differentiated from resistant callus. The T indicated by the arrow was mutated from G, resulting in a W2038L mutation.

FIG. 17 shows the resistance test of T1 generation OsACCase2 W2038L edited seedlings to Haloxyfop-p. From left to right, seedling No. 1 shows the results of the water treatment control of wild-type Huaidao No. 5 (a rice variety), and seedlings Nos. 2 to 4 show the results of wild-type Huaidao No. 5 and two RNP gene gun-transformed T1 generation W2038L edited strains QY367-7-12 and QY367-7-18, all of which were treated with 5 g/mu active ingredient of Haloxyfop-p (mu, a unit of area, 1 mu=1/15 hectares). It is clearly that the edited strains developed resistance to Haloxyfop-p.

FIG. 18 shows the resistance test of T1 generation OsACCase2 W2038L edited seedlings to quizalofop-p. From left to right, seedling No. 1 shows the results of the water treatment control of wild-type Huaidao No. 5, and seedlings Nos. 2 to 4 show the results of wild-type Huaidao No. 5 and two RNP gene gun-transformed T1 generation W2038L edited strains QY367-5-10 and QY367-5-21, all of which were treated with 5 g/mu active ingredient of quizalofop-p. It is clearly that the edited strains developed resistance to quizalofop-p.

FIG. 19 shows the resistant callus of Oryza sativa edited by RNP simultaneously at OsACCase2 W2038 site and OsBADH2 gene and screened with 50 ug/L quizalofop-p. The arrow indicates the resistant callus.

FIG. 20 shows a diagram of sequencing peaks of OsACCase2 W2038 target site of TO generation double-site edited seedlings. The T indicated by the arrow was mutated from G, resulting in a W2038L mutation.

FIG. 21 shows a diagram of sequencing peaks of OsBADH2 target site of TO generation double-site edited seedling. +A homozygous mutation occurred at the point indicated by an arrow.

FIG. 22 shows the resistant callus of Oryza sativa edited by RNP simultaneously at OsALS W548 site and OsSWEET14 gene and screened with 5 mg/L pyroxsulam. The arrow indicates the resistant callus.

FIG. 23 shows a diagram of sequencing peaks of OsALS W548 target site of TO generation double-site edited seedlings. Both G base and T base signal peaks are present at the place indicated by an arrow, resulting in W548L mutation.

FIG. 24 shows a diagram of sequencing peaks of OsSWEET14 target site of TO generation double-site edited seedlings. −C homozygous mutation occurred at the site indicated by an arrow.

FIG. 25 shows the resistance test of T1 generation OsALS W548 site and OsSWEET14 double-site edited seedlings to nicosulfuron. From left to right, seedling No. 1 shows the results of the water treatment control of wild-type Huaidao No. 5, and seedlings Nos. 2 to 3 show the results of the wild-type Huaidao No. 5 and the RNP gene gun-transformed T1 generation W548L edited strain QY360-7-11, both of which were treated with 4 g/mu active ingredient of nicosulfuron. It is clearly that the edited strain developed resistance to nicosulfuron.

FIG. 26 shows the resistance test of T1 generation OsALS W548 site and OsSWEET14 double-site edited seedlings to flucarbazone-Na. From left to right, seedling No. 1 shows the results of the water treatment control of wild-type Huaidao No. 5, and seedlings Nos. 2 to 3 show the results of the wild-type Huaidao No. 5 and the RNP gene gun-transformed T1 generation W548L edited strain QY360-7-9, both of which were treated with 2 g/mu active ingredient of flucarbazone-Na. It is clearly that the edited strain developed resistance to flucarbazone-Na.

FIG. 27 shows the resistance test of T1 generation OsALS W548 site and OsSWEET14 double-site edited seedlings to imazapic. From left to right, seedling No. 1 shows the results of the water treatment control of wild-type Huaidao No. 5, and seedlings Nos. 2 to 3 show the results of the wild-type Huaidao No. 5 and the RNP gene gun-transformed T1 generation W548L edited strain QY360-7-11, both of which were treated with 7 g/mu active ingredient of imazapic. It is clearly that the edited strain developed resistance to imazapic.

FIG. 28 shows the resistance test of T1 generation OsALS W548 site and OsSWEET14 double-site edited seedlings to pyroxsulam. From left to right, seedling No. 1 shows the results of the water treatment control of wild-type Huaidao No. 5, and seedlings Nos. 2 to 4 show the results of the wild-type Huaidao No. 5 and the RNP RNP gene gun-transformed T1 generation W548L edited strains QY360-7-2 and QY360-7-11, all of which were treated with 2 g/mu active ingredient of pyroxsulam. It is clearly that the edited strains developed resistance to pyroxsulam.

FIG. 29 shows a scheme of programmed sequential cutting/editing of HBB gene in 293T cells. A. The design of HBB gene sites for programmed sequential cutting/editing in 293T cells is shown; B. The conversion efficiency of programmed sequential cutting/editing of HBB gene in 293T cells after 48 hours of transformation of each editing vector; C. The ratio of gene editing types generated from programmed sequential cutting/editing of HBB gene and single site cutting/editing of HBB gene in 293T cells; WT: wild type, indel: a genotype for deletion or insertion, C->T SNP: a genotype with C to T base substitution at cutting.

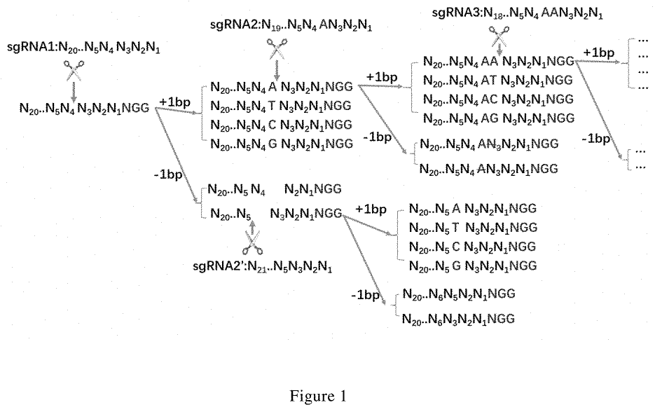

### DESCRIPTION OF SEQUENCE LISTING

The main sequences involved in the present invention are summarized as follows, and related sequences are provided in the sequence listing.

| SEQ ID NO: | Sequence description |
|---|---|
| 1 | Amino acid sequence of ALS protein from Arabidopsis thaliana |
| 2 | DNA sequence of ALS gene from Arabidopsis thaliana |
| 3 | Amino acid sequence of AmACCase protein from Alopecurus |
| | myosuroides |
| 4 | DNA sequence of AmACCase gene from Alopecurus myosuroides |
| 5 | Amino acid sequence of OsHPPD protein from Oryza sativa |
| 6 | Genomic DNA sequence of OsHPPD protein from Oryza sativa |
| 7 | Amino acid sequence of OsPPO1 protein from Oryza sativa |
| 8 | Genomic DNA sequence of OsPPO1 protein from Oryza sativa |
| 9 | Amino acid sequence of OsTIR1 protein from Oryza sativa |
| 10 | Genomic DNA sequence of OsTIR1 protein from Oryza sativa |
| 11 | Amino acid sequence of ALS protein from Oryza sativa |
| 12 | DNA sequence of ALS gene from Oryza sativa |
| 13 | Amino acid sequence of ACCase2 protein from Oryza sativa |
| 14 | Genomic DNA sequence of ACCase2 from Oryza sativa |
| 15 | DNA sequence of SpCas9 protein optimized by plant codon |
| 16 | DNA sequence of NGA-Cas9 protein optimized by plant codon |
| 17 | Genomic DNA sequence of OsBADH2 from Oryza sativa |
| 18 | Genomic DNA sequence of OsSWEET14 from Oryza sativa |
| 19 | Amino acid sequence of StALS2 protein from potato (Solanum tuberosum L.) |
| 20 | DNA sequence of StALS2 gene from potato (Solanum tuberosum L.) |
| 21 | DNA sequence of HBB (hemoglobin subunit beta) gene in human |
| | embryonic kidney cell 293T |
| 22 | CDS sequence of HBB gene in human embryonic kidney cell 293T |
| 23 | Amino acid sequence of HBB gene in human embryonic kidney cell 293T |

### Embodiments for Carrying Out the Invention

The present invention will be further explained in conjunction with the following examples. The following examples are illustrated by way of examples, but the protection scope of the present invention should not be limited to these examples. The experimental methods in the following examples, unless otherwise specified, were the methods described in commonly used molecular biology, tissue culture technology and agronomy manuals. For example, specific steps could be found in: “Molecular Cloning: A Laboratory Manual (3rd edition)” (Sambrook, J., Russell, David W., 2001, Cold Spring Harbor), “Plant Propagation by Tissue Culture” (Edwin F. George, Michael A. Hall, Geert-Jan De Klerk, 2008, Springer). The materials, reagents, instruments, etc. used in the following examples could be obtained from commercial sources unless otherwise specified.

### Example 1: Designing Predictable Base Substitutions Introduced by Programmed Sequential Cutting/Editing for W574 and 5653 Sites of ALS Gene of Arabidopsis thaliana

A. Experimental Materials

1. Arabidopsis thaliana Material

The wild-type Arabidopsis thaliana Col-0 was a model variety of dicotyledonous plant, its original seeds were provided by the Department of Weeds, College of Plant Protection, China Agricultural University, and the propagation and preservation thereof were performed by our laboratory according to standard methods in this field.

2. Vectors

The vector plasmids pCBC-dT1T2 (Xing H L, Dong L, Wang Z P, Zhang H Y, Han C Y, Liu B, Wang X C, Chen Q J 2014. A CRISPR/Cas9 toolkit for multiplex genome editing in plants. BMC Plant Biol. November 29; 14(1):327, see https://www.addgene.org/50590/for details), pHEE401E (see Wang Z P, Xing H L, Dong L, Zhang H Y, Han C Y, Wang X C, Chen Q J Genome Biol. 2015 Jul. 21; 16:144. doi: 10.1186/s13059-015-0715-0. https://www.addgene.org/71287/for specific information), and pHEE401E-NG (the mutation as reported in Nishimasu et al. 2018 Engineered CRISPR-Cas9 nuclease with expanded Targeting space. Science 361(6408):1259-1262. doi: 10.1126/science.aas9129 was introduced into pHEE401E to construct vector pHEE401E-NG capable of recognizing NG PAM) were purchased from Addgene website or constructed by our laboratory in accordance with conventional molecular biology methods, and kept by our laboratory.

3. Main Equipment

Pipette gun, water bath, PCR instrument (Bio-rad T100), electrophoresis instrument (WIX-EP600), gel imager, electric blast dryer, centrifuge (Eppendorf 5424R), high-throughput tissue lyser, shaker, electronic balance, pH meter, etc.

4. Main Reagents

High-fidelity DNA polymerase (purchased from Tsingke Bio), agarose gel recovery kit and plasmid extraction kit (purchased from Sparkjade), BsaI and T4 DNA ligases (purchased from NEB), Trans5α competent cells and EHA105 competent cells (purchased from TransGen Biotech, Beijing, China), GV3101 Agrobacterium competent cells (purchased from Shanghai AngyuBio), Tris, EDTA, kanamycin, cephalosporin, hygromycin, agarose, yeast powder, tryptone, NaCl (purchased from Sangon Biotech), MS powder, sucrose, Silwet-77, hygromycin (purchased from Solarbio), nucleic acid dye (Dured), absolute ethanol (purchased from Sinopharm), etc.

5. Preparation of Main Solutions
1) Seed disinfectant: 4 mL of 10% SDS, 20 mL NaClO, added with water to make up 200 mL.
2) SDS extraction buffer: 40 mL of 1M Tris HCl (pH 8.0), 50 mL of 0.1M EDTA, 10 mL of 5M NaCl, 10 mL of 10% SDS, added with water to make up 200 mL.
3) Infection solution: 1.5 g of sucrose, 9 μL of Silwet-77, added with 30 mL of ultrapure water.
4) LB solid medium: 5 g of yeast powder, 10 g of tryptone, 10 g of NaCl, 15 g of agar, added with water to make up 1 L, sterilize at 121° C. for 15 minutes, poured on a plate for later use.
5) 50×TAE stock: 242 g of Tris, 37.2 g of Na₂EDTA·2H₂O, added with 800 mL of ultrapure water, stirred well to dissolve, added with 57.1 mL of acetic acid, stirred well, finally diluted to 1 L with deionized water, and stored at room temperature.
6) MS solid medium: 4.42 g of MS powder and 10 g of sucrose were weighed, added with 800 mL of ultrapure water, adjusted to pH 5.8, added with water to make up 1 L, added 10 g of phytagel, sterilized at 121° C. for 15 minutes, and poured on a plate for later use.

B. Experimental Methods

1. Design and Construction of CRISPR/Cas9 Dual Target Vector

1.1 Target Design

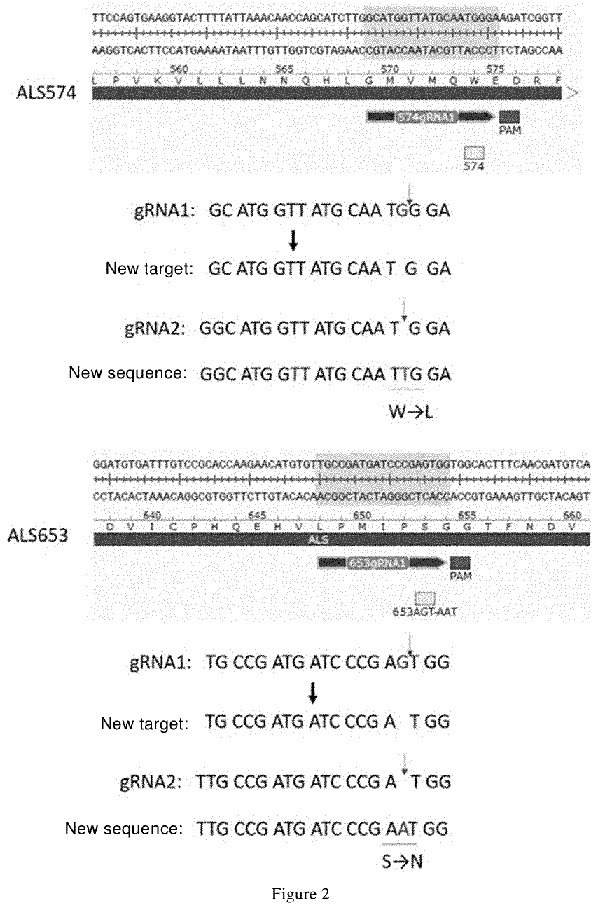

The Arabidopsis thaliana ALS gene sequence was shown in SEQ ID NO: 2. The target sequence gRNA1 (5′-GCATGGTTATGCAATGGGA-3′) of 19 bases was designed using the AGA near Arabidopsis thaliana ALS574 site as PAM, it was predicted that one G base would be deleted between the first 3-4 sites of PAM after editing, then a second target sequence gRNA2 (5′-GGCATGGTTATGCAATGGA-3′) was designed based on the sequence generated from the deletion, and it was predicted that one T base would be inserted via a second editing, thereby realizing conversion of TGG-TTG, as shown in FIG. 2.

Similarly, the target sequence gRNA3 (5′-TGCCGATGATCCCGAGTGG-3′) of 19 bases was designed using the TGG near Arabidopsis thaliana ALS653 site as PAM, it was predicted that one G base would be deleted between the first 3-4 positions of PAM after editing, then a second target sequence gRNA4 (5′-TTGCCGATGATCCCGATGG-3′) was designed based on the sequence generated from the deletion, and it was predicted that one A base would be inserted after the second editing, thereby realizing conversion of AGT-AAT, as shown in FIG. 2.

1.2 Vector Construction

The method described by Xing H L, Dong L, Wang Z P, Zhang H Y, Han C Y, Liu B, Wang X C, Chen Q J 2014. A CRISPR/Cas9 toolkit for multiplex genome editing in plants. BMC Plant Biol. November 29; 14(1): 327 was followed. Specifically, dT1T2 plasmid was used as a template to amplify the ALS574 and 653 sites dual-target fragment respectively to construct an sgRNA expression cassette. The vector backbones of pHEE401E and pHEE401E-NG were digested with BsaI, and the bands were cut from the gel and recovered, and the target fragment was directly used for the ligation reaction after digestion. T4 DNA ligase was used to ligate the vector backbones and the target fragment, the ligation products were transformed into Trans5a competent cells, different monoclones were picked out for sequencing. After confirmation via sequencing, the Sparkjade High Purity Plasmid Mini Extraction Kit was used to extract the plasmids to obtain the recombinant plasmids, which were respectively named as pQY743 and pQY745.

2. Design of Primers for Target Detection

Primers for target detection took ALS574 and ALS653 target sites as centers, wherein the primer for upstream detection was about 100 bp from the ALS574 target site, and the primer for downstream detection was about 280 bp from the ALS653 target site. The primer sequences were as follows: 574/653checking-F: 5′ATTGACGGAGATGGAAGCTT3′, and 574/653checking-R: 5′CCAAACTGAGCCAGTCACAA3′.

3. Establishment of Arabidopsis thaliana Genetic Transformation System

3.1 Agrobacterium Transformation

The constructed recombinant plasmids were transformed into Agrobacterium GV3101 competent cells to obtain recombinant Agrobacterium cells.

3.2 Preparation of Agrobacterium Infection Solution
1) The activated Agrobacterium was picked and inoculated in 30 ml of YEP liquid medium (containing 25 mg/L Rif and 50 mg/L Kan), and cultured under shaking at 200 rpm and 28° C. overnight until the OD600 value was about 1.0-1.5.
2) After centrifugation at 6000 rpm for 10 minutes, the Agrobacterium was collected, and the supernatant was discarded.
3) The Agrobacterium was resuspended in the infection solution (no need to adjust pH) to OD600=0.8 for later use.

3.3 Transformation of Arabidopsis thaliana
1) Before plant transformation, the attention was paid to whether the plant grew well, the inflorescence was luxuriant, and there was no stress response. The first transformation was carried out when plant height was about 20 cm. Watering could be properly carried out when the soil was dry. The grown siliques were cut off with scissors on the day before transformation.
2) The inflorescence of the plant to be transformed was immersed in the above solution for 30 seconds to 1 minute under gently stirring, and there should be a layer of liquid membrane on the infiltrated plant.
3) After the transformation was completed, the plant was cultured in the dark for 24 hours, and then taken out and placed in a normal light environment for growth.
4) After one week, the second transformation could be carried out in the same way.

3.4 Seed Harvest

After the seeds were mature, they were harvested. After harvesting, the seeds were dried in an oven at 37° C. for about one week.

4. Selection of Transgenic Plant

The seeds were treated with disinfectant for 5 minutes, washed with deionized water for 5 times, and evenly spread on the MS selection medium (containing 30 μg/mL hygromycin, 100 μg/mL cephalosporin), the medium was placed in a light incubator (temperature 22° C., 16 hours light, 8 hours dark, light intensity 100-150 μmol/m²/s, humidity 75%), and one week later, the positive seedlings were selected and transplanted to soil.

5. Detection of T1 Mutant Plants

5.1 Extraction of Genomic DNA
1) The leaves of Arabidopsis thaliana were cut and placed in a 2 mL centrifuge tube, added with steel balls, and the leaves were ground with the high-throughput tissue lyser.
2) After the grinding was complete, 400 μL of SDS extraction buffer was added, mixed upside down, and incubated in a 65° C. water bath for 15 minutes and mixed upside down every 5 minutes.
3) Centrifugation was carried out at 13,000 rpm for 5 minutes.
4) 300 μL of the supernatant was pipetted and transferred to a new 1.5 mL centrifuge tube, an equal volume of isopropanol pre-cooled at −20° C. was added to the centrifuge tube, and the centrifuge tube was placed at −20° C. for 1 hour or overnight.
5) Centrifugation was carried out at 13,000 rpm for 10 minutes and the supernatant was discarded.
6) 500 μL of 70% ethanol was added to the centrifuge tube to wash the pellet, the washing solution was discarded after centrifugation (be careful not to discard the pellet), dried at room temperature, 30 μL of ultrapure water was added to dissolve the DNA, and the DNA was stored at −20° C.

5.2 PCR Amplification

The extracted T1 plant genome was used as a template, the detection primers were used to amplify target fragment, 5 μL of amplified product was pipetted and detected by 1% agarose gel electrophoresis, and imaged with the gel imager. The remaining product was delivered to the sequencing company to directly perform sequencing.

6. Detection of T2 Mutant Plant

After the seeds of the T1 strain were harvested from single plant, the seeds of different strains of two vectors were selected and spread on the imazapic selection medium (MS medium+0.24 μg/mL imazapic) to perform selection, and the positive seedlings were transplanted to the soil one week later and subjected to molecular detection, in which the method was the same as step 5.

The used primers and sequences thereof:

| Primer name | Sequence (5′-3′) |
|---|---|
| ALS574-F | ATATATGGTCTCGATTGGCATGGTTAT |
| | GCAATGGGAGTTTTAGAGCTAGAAATA |
| ALS574-R | ATTATTGGTCTCGAAACTCCATTGCAT |
| | AACCATGCCCAATCTCTTAGTCGACTC |
| ALS653-F | ATATATGGTCTCGATTGTGCCGATGAT |
| | CCCGAGTGGGTTTTAGAGCTAGAAATA |
| ALS653-R | ATTATTGGTCTCGAAACCCATCGGGAT |
| | CATCGGCAACAATCTCTTAGTCGACTC |
| ALS574/653 | ATTGACGGAGATGGAAGCTT |
| checking-F | |
| ALS574/653 | CCAAACTGAGCCAGTCACAA |
| checking-R | |

C. Experimental Results

1. Genotype Detection of T1 Plant

T1 seeds were selected by MS hygromycin resistance medium, a total of 32 positive seedlings were obtained for the pQY743 vector, and a total of 18 positive seedlings were obtained for the pQY745 vector. For each vector, 10 seedlings were selected and leaf genomic DNA thereof was extracted to detect the target site. It was found that there was no editing occurred in the T1 generation at ALS574 site, and there were editing events that met the design expectations at ALS653 site. The detection results are shown in Table 1:

TABLE 1
Mutation types of T1 plants

| ALS site | Vector number | T1 plant number | Edit type |
|---|---|---|---|
| 574 | pQY743 | 1-10 | WT |
| 653 | pQY745 | 3, 4-8, 10 | Chimera |
| | | 1, 2, 9 | Heterozygote |

2. Selection Results of T2 Generation Seeds

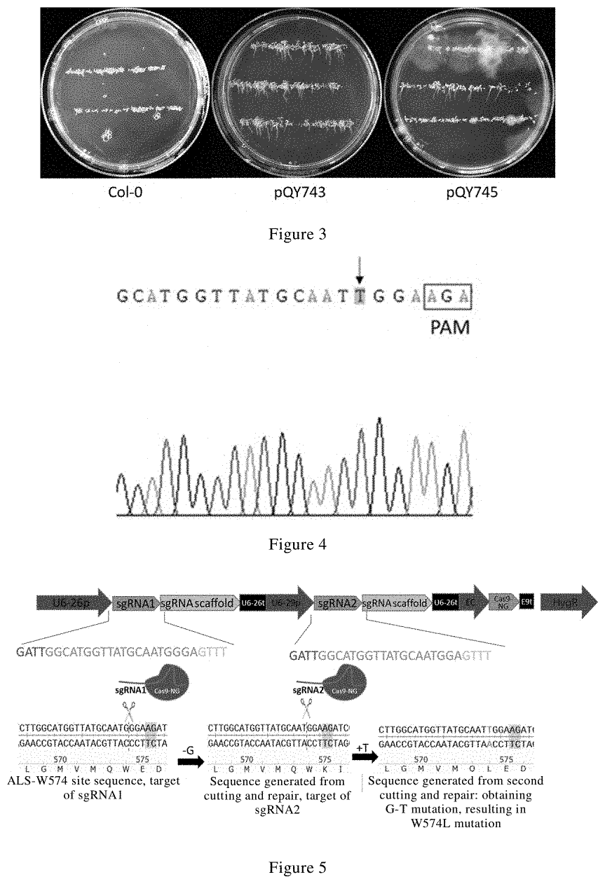

After the T2 generation seeds of single plant were harvested, they are spread on the imazapic resistant medium to perform selection, it could be seen that the wild-type Col-0 could not grow on the resistant medium, while the positive plants of the mutant strains could grow normally on the resistant medium, as shown in FIG. 3.

For each vector, 10 seedlings were selected and genomic DNA thereof was extracted from leaves for molecular detection. It was found that there were 6 strains for the pQY743 vector that had homozygous mutation in line with the expectations, i.e., mutation from TGG to TTG, as shown in FIG. 4. In addition, there was 1 strain with heterozygous mutation, and there were 3 strains with chimeric mutation. The detection results are shown in Table 2.

TABLE 2
Mutation types of T2 plants

| ALS site | Vector No. | T2 plant No. | Edit type |
|---|---|---|---|
| 574 | pQY743 | 2, 3, 5, 6, 7, 9 | Homozygote |
| | | 4 | Heterozygote |
| | | 1, 8, 10 | Chimera |

The above results showed that by using the technical solution of programmed sequential cutting/editing of the present invention, the design and realization of expected mutations at the target site could be achieved, and the base substitution mutations could be realized by designing sequential sgRNA combinations only with Cas9 protein.

### Example 2: Achieving Multiple Mutation Types by Programmed Sequential Cutting/Editing at W574 and S653 Sites of Arabidopsis thaliana ALS Gene

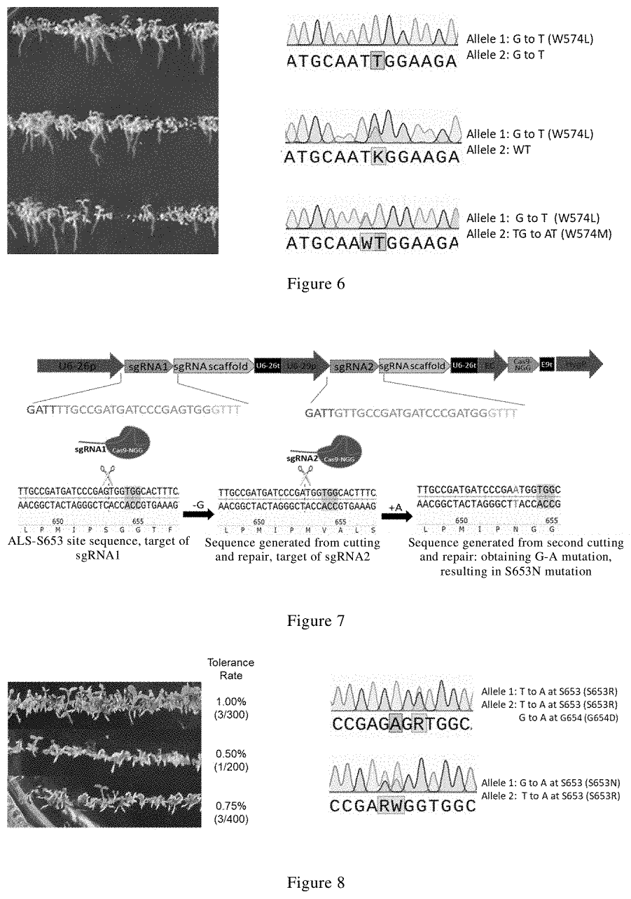

The operation steps for vector design, construction, and Arabidopsis thaliana transformation and selection were performed by referring to Example 1. For AtALS W574 site, the vector design was the same as that of Example 1. The schematic diagram of the vector was shown in FIG. 5, and it was expected that the W574L mutation was to be realized by first −G then +T at a specific site. No editing event was detected in the T1 generation of the vector transformed Arabidopsis thaliana. The T2 generation of the transgenic Arabidopsis thaliana was selected with 0.24 mg/L imazapic, and a large number of herbicide-resistant plants were obtained, as shown in the left panel of FIG. 6, the molecular detection of these resistant plants was performed, the PCR product sequencing results showed that not only the expected W574L mutation appeared at the W574 site, but also another resistant mutation W574M appeared, as shown in the right panel FIG. 6.

For the AtALS S653 site, the vector design was the same as that of Example 1. The vector diagram was shown in FIG. 7, and it was expected that the S653N mutation was to be realized by first −G then +A at a specific site. An expected editing event of S653N was detected in the T1 generation of the vector transformed Arabidopsis thaliana. The T2 generation of the transgenic Arabidopsis thaliana was continuously selected with 0.24 mg/L imazapic, and a large number of herbicide-resistant plants were obtained, as shown in the left panel of FIG. 8, molecular detection was performed to these resistant plants, the PCR product sequencing results showed that not only the expected S653N mutation occurred at S653 site, but also two other resistant mutations S653R and S653R/G654D occurred, as shown in the right panel of FIG. 8.

The above results showed that by using the technical solution of programmed sequential cutting/editing of the present invention, in addition to the expected mutations designed for the target site, multiple functional mutation types could also be generated by designing sequential sgRNA combinations with only corresponding Cas9 protein, so that it is a suitable tool for creating new functional mutations.

### Example 3: Generating W574M Resistance Mutation by Using Different PAMs to Perform Programmed Sequential Cutting/Editing Near the W574 Site of Arabidopsis thaliana ALS Gene

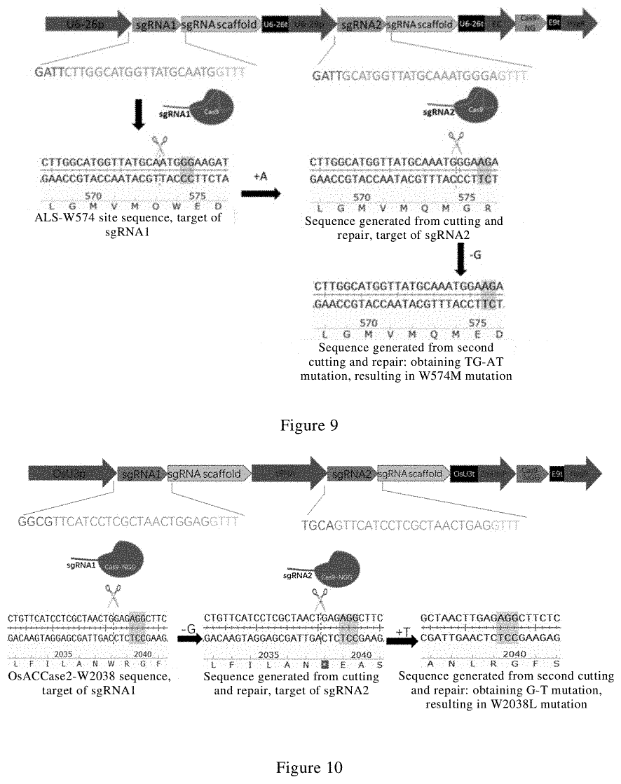

The operation steps for vector design, construction, and Arabidopsis thaliana transformation and selection were performed by referring to Example 1, except that, for the sequence 5′CTTGGCATGGTTATGCAATGgg3′ near the AtALS W574 site, the GG closer to the W574 site was used first as PAM to design sgRNA1: 5′CTTGGCATGGTTATGCAATG3′, in which the W574 site was underlined and the NG PAM was shown in italics. It was predicted that a new sequence 5′CTTGGCATGGTTATGCAAATGGGAag3′ was to be formed by +A after cutting and repair. The AG was used as a new PAM site to design sgRNA2: 5′CTTGGCATGGTTATGCAAATGGGA3′, and −G genotype was formed after spontaneous repair of cells, resulting in W574M. That was, different PAM sites were used for the two cuttings in this scheme. The vector diagram was shown in FIG. 9. The vector was used to transform Arabidopsis thaliana, the T1 generation transgenic strain was subjected to genotype detection from which the expected editing event of W574M was detected, and the plant showed resistance to imazapic treatment.

The results showed that by using the technical solution of the present invention and using different PAMs to perform programmed sequential cutting/editing, the editing could be carried out in a wider sequence range to obtain amino acid substitution.

### Example 4: Designing Predictable Base Substitutions for D350 and W548 Sites of Oryza sativa ALS Gene

The mutation as reported in Nishimasu et al. 2018 Engineered CRISPR-Cas9 nuclease with expanded targeting space. Science 361(6408):1259-1262. doi: 10.1126/science.aas9129 was introduced into pHUE411 (A CRISPR/Cas9 toolkit for multiplex genome editing in plants. Xing H L, Dong L, Wang Z P, Zhang H Y, Han C Y, Liu B, Wang X C, Chen Q J. BMC Plant Biol. 2014 Nov. 29; 14(1):327. 10.1186/s12870-014-0327-y, see details in https://www.addgene.org/71287/) to construct the vector pHUE411-NG capable of recognizing NG PAM.

The Oryza sativa ALS gene sequence was shown in SEQ ID NO: 12. By using 5′GGCGTGCGGTTTGATGATCG3′ (the underlined part was the OsALS-D350 site corresponding to Arabidopsis thaliana ALS-D376) and a new sequence 5′GGCGTGCGGTTTGATGACG3′ that was predicted to be generated from editing as targets, a dual-target vector was constructed according to the method described in Xing H L, Dong L, Wang Z P, Zhang H Y, Han C Y, Liu B, Wang X C, Chen Q J. BMC Plant Biol. 2014, and it was expected to obtain the conversion of GAT-GAA, resulting in OsALS D350E mutation.

By using 5′GGTATGGTTGTGCAATGGGA3′ (the underlined part was the OsALS-W548 site corresponding to Arabidopsis thaliana ALS-W574) and a new sequence 5′GGTATGGTTGTGCAATGGA 3′ that was predicted to be generated from editing as targets, a dual-target vector was constructed, and it was expected to obtain the conversion of TGG-TTG, resulting in OsALS W548L mutation.

Then the two vectors were transferred into Oryza sativa to obtain transgenic plants, and the identification indicated that the plants with expected substitutions D350E and W548L were obtained. The results of herbicide resistance biotest in field showed that the D350E and W548L mutants acquired the resistance to ALS inhibitor herbicides.

### Example 5: Designing Predictable Base Substitutions and Selecting Multiple Mutation Types for W2038 Site of Oryza sativa ACCase2 Gene

The Oryza sativa ACCase2 gene sequence was shown in SEQ ID NO: 14, in which OsACCase2 W2038 site corresponded to the ACCase W2027 site of Alopecurus myosuroides. The AGG close to this site was used as PAM to design sgRNA1: 5′TTCATCCTCGCTAAC-TGAG3′, and it was predicted that a new sequence was formed by −G after cutting and repair. The AGG was continuously used as PAM to design sgRNA2: 5′CTTC-ATCCTCGCTAACTGAG3′, and +T genotype was formed after cutting and repair again, resulting in the W2038L mutation. The sgRNA1 and sgRNA2 were constructed on the pHUE411 vector to form an editing vector, and the vector diagram was shown in FIG. 10.

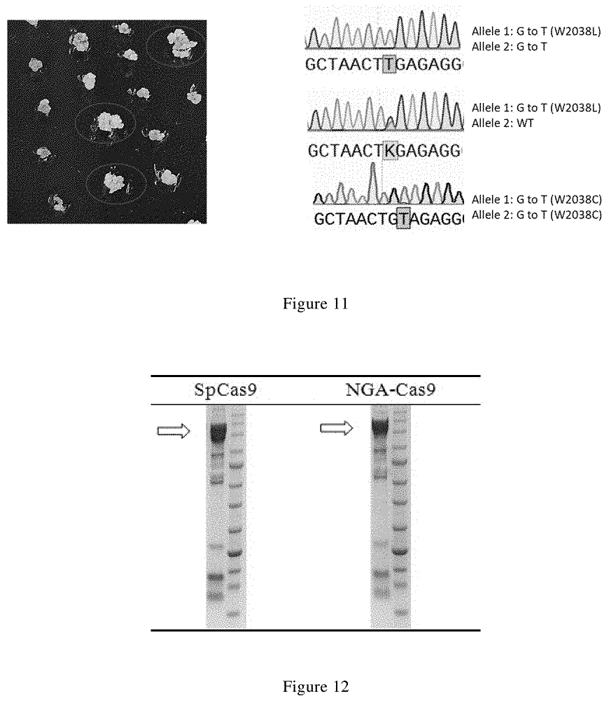

The editing vector was used to transform the callus of Huaidao No 5 (a rice variety), and after 3 weeks of co-selection with 50 μg/L hygromycin and 50 μg/L quizalop-p, a large number of resistant calli were obtained, as shown on the left panel of FIG. 11. The resistant callus was taken for genotype identification, and it was found that not only the expected W2038L mutation but also the W2038C mutation occurred, as shown on the right panel of FIG. 11.

The above results of programmed sequential cutting/editing of Oryza sativa gene showed that the technical solution of the present invention was applicable to both monocotyledonous and dicotyledonous plants.

### Example 6: Expression and Purification of SpCas9 and NGA-Cas9 Proteins

1. Experimental Instrument and Reagents

TABLE 3
Experimental reagents

| Reagent | Preparation and usage |
|---|---|
| Q5 DNA polymerase | Purchased from NEB |
| I5 DNA polymerase and I5 reaction | Purchased from Tsingke Bio |
| solution | |
| dNTPs | Shanghai Sangon |
| Ni-NTA resin | Shanghai Sangon |
| Tris/NaCl/imidazole | Shanghai Sangong |
| DTT | Shanghai Sangong |
| β-mercaptoethanol | Sigma |
| SDS-PAGE precast gel | Nanjing GenScript |
| Superdex200 column | GE Healthcare |

TABLE 4
Experimental Instruments

| Instrument | Model |
|---|---|
| PCR instrument | Bio-rad T100 |
| Constant temperature shaking bed | Ounuo HNY-200B |
| Cell disrupter | Ningbo Xinzhi JY92-IIN |
| Electrophoresis apparatus | WIX-EP600 |
| Protein purifier | GE AKTA pure |
| High speed large capacity refrigerated | Xiangzhi ZX21K |
| centrifuge | |
| High-speed centrifuge | Eppendorf 5424R |
| White light film viewer | Beijing Liuyi WD-9406 |
| Nucleic acid gel imager | Beijing Sage ChampGel ™ 5000 |
| Micro spectrophotometer | DS-II |

2. Experimental Method

2.1 Construction of pET15b-Cas9 Expression Vector

The DNA sequences of SpCas9 and NGA-Cas9 proteins obtained after plant codon optimization were shown in SEQ ID NO: 15 and SEQ ID NO: 16, respectively, and the sequences were synthesized by GenScript as template DNAs.

After the NG-Cas9 and NGA-Cas9 sequences were amplified separately, the two fragments were ligated to a pET15b expression vector by infusion method, transformed into DH5a, and sequenced after verification.

TABLE 5
PCR verification amplification
system and primers

| | Reaction system, 50 ul |
|---|---|
| 2× I5 reaction | 25 ul |
| solution | |
| Template DNA | 1 ul |
| Forward and | 2 ul each |
| reverse primers | |
| Ultrapure water | Added to 50 ul |
| Primer sequence: | 5′-3′ |
| pET15b-NG Cas9-F | tgccgcgcggcagccatatga |
| | tggattacaaggaccacgacg |
| pET15b-NG Cas9-R | tttgttagcagccggatcctc |
| | acttcttcttcttggcctgtc |
| | cc |
| pET15b-NG Cas9-F | gtgccgcgcggcagccatatg |
| | gattacaaggaccacgacg |
| pET15b-NG Cas9-R | tttgttagcagccggatcctc |
| | acttcttcttcttcgcctgc |

2.2 Expression and Purification of Proteins

The constructed expression vectors were transformed into Escherichia coli Rosetta (DE3), the expression thereof was induced by IPTG, and the bacteria were harvested, lyzed and purified by Ni-NTA column. The specific method was as follows:
a) The recombinant expression vectors were transformed into Rosetta (DE3) strain, a single colony was picked into 10 ml of LB medium, CmR+Amp (pET15b) or CmR+Kana (pET28a) resistance, cultured at 200 rpm at 37° C. overnight, transferred to a 2 L shake flask containing 1 L of LB medium, cultured at 37° C. and 200 rpm until the OD600 reached 0.6-0.8, cooled to 18° C., the expression thereof was induced with 0.5 mM IPTG overnight, and the bacteria were harvested by centrifugation at 4000 g.
b) The harvested bacteria were resuspended in Ni-buffer A: 50 mM HEPES, pH7.4, 500 mM NaCl, 20 mM imidazole, 5 mM β-mercaptoethanol, added with PMSF with a final concentration of 1 mM and 250 ul of Cocktail inhibitor, and then mixed well.
c) After the resuspended cells were disrupted by an ultrasonic disruptor, they are centrifuged at 40000 g for 30 minutes at 4° C., and the supernatant was collected and passed through a Ni-NTA column.
d) Purification via Ni-NTA column: the lysate supernatant was combined with resin for 20 minutes, eluted with buffer A containing 50 mM imidazole to remove impurities, and finally eluted with an elution buffer containing 400 mM imidazole.
e) SDS-PAGE gel electrophoresis system was used to detect the purification effect of protein.
f) Dialysis was performed by changing the buffer to 50 mM HEPES pH 7.5, 150 mM KCl, 1 mM DTT, 3% glycerol.
h) The final sample was subjected to SDS-PAGE gel electrophoresis to detect the purification effect of the protein. After concentration by ultrafiltration, the protein was frozen at −80° C. for later use.

2.3 Expression and Purification Results of Cas9 Fusion Proteins

The purification results of SpCas9 and NGA-Cas9 proteins were shown in FIG. 12. The arrows indicated the Cas9 protein bands, indicating that a higher purity was achieved.

### Example 7: In Vitro Enzyme Cleavage Activity of SpCas9 and NGA Cas9 Proteins Separately Detected to OsACCase2 W2038 and OsALS W548 Target Sites

1. The DNA sequences of OsALS and OsACCase2 genes were input into the CRISPOR online tool (http://crispor.tefor.net/), the following sgRNAs were designed for the W548 of OsALS (the 548th amino acid of OsALS, the amino acid sequence of Oryza sativa ALS protein was shown in SEQ ID NO: 11) corresponding to the amino acid site 574 of Arabidopsis thaliana ALS protein and the W2038 of OsACCase2 (the 2038th amino acid of OsACCase2, the amino acid sequence of Oryza sativa ACCase2 protein was shown in SEQ ID NO: 13) corresponding to the amino acid site 2027 of Alopecurus myosuroides, and these sgRNAs were synthesized by GenScript:

>sgRNA1-548-G:
5′GGGUAUGGUUGUGCAAUGGGAguuuuagagcu
agaaauagcaaguuaaaauaaggcuaguccguua
ucaacuugaaaaaguggcaccgagucggugc3′
> sgRNA1-2038-G:
5′GUUCAUCCUCGCUAACUGGAGguuuuagagcu
agaaauagcaaguuaaaauaaggcuaguccguua
ucaacuugaaaaaguggcaccgagucggugc3′

2. The specific detection primers, OsALS265AA-F: 5′ggtcttgcgtctggttggc3′ and OsALS-end-R: 5′ccatgccaagcacatcaaacaag3′, were used to amplify the fragment containing the OsALS W548 target site, and the PCR product was 1200 bp in length.

The specific detection primers, OsACC1750AA-F: 5′gcgaagaagactatgctcgtattgg3′ and OsACC2196AA-R: 5′cttaatcacacctttcgcagcc3′, were used to amplify the fragment containing the OsACCase W2038 target site, and the PCR product was 1500 bp in length.

The PCR system was shown in the following table:

| Component | Volume |
|---|---|
| 2× I5 reaction solution | 25 μL |
| Forward primer (10 μM) | 2 μL |
| Reverse primer (10 μM) | 2 μL |
| Genomic DNA template | 2 μL |
| Ultrapure water | Added to 50 μL |

3. The PCR reaction was established, and the reaction conditions were shown in the following table:

| Step | Temperature | Time | |
|---|---|---|---|
| Pre-denaturation | 98° C. | 30 | s |
| 30-35 amplification | 98° C. | 15 | s |
| cycles | 58° C. | 15 | s |
| | 72° C. | 30 | s |
| Final extension | 72° C. | 3 | min |

4. The PCR products were detected by agarose gel electrophoresis and subjected to sequencing for further verification. After being correctly verified, the gel was cut to recover DNA fragments. The recovered DNA fragments were dissolved by adding 30 ul of RNase-free ultrapure water, and the concentration thereof was measured.
5. The following detection system was used to detect Cas9 protein activity, and after the system was prepared, it was incubated at 37° C. for 1 hour.

| Components | Volume | |
|---|---|---|
| Cas9 protein | 0.5 | μL (1 ug) |
| sgRNA | 0.5 | μL (1 ug) |
| 10× Cas9 reaction buffer | 2 | μL |
| Recovered DNA fragment | X | μL (100 ng) |

| | |
|---|---|
| RNase-free ultrapure water | Added to 20 μL |

6. After the reaction was completed, the system was treated at 65° C. for 10 minutes, added with 4 ul of 6×DNA loading buffer, and run on 2% agarose gel to detect band size.

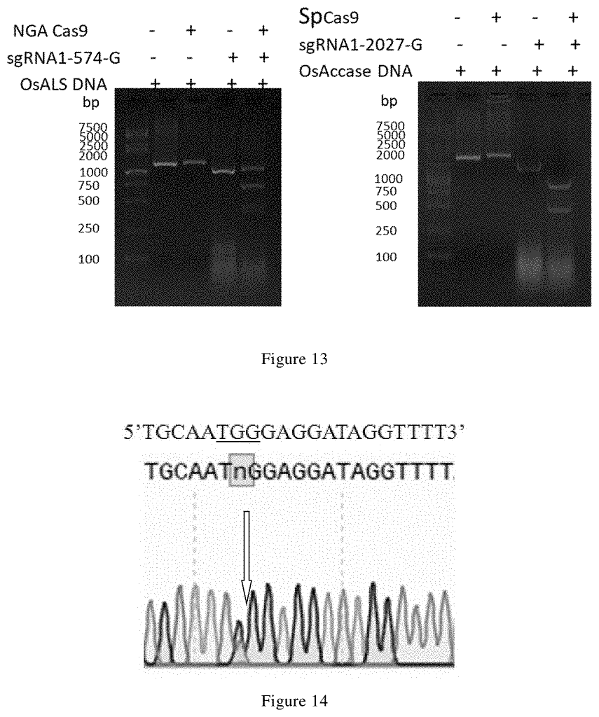

The results were shown in FIG. 13, and it can be seen that the purified Cas9 proteins could cut DNA double-strand at the designed target sites only in the presence of sgRNA.

### Example 8: Achieving W548L Mutation at OsALS548 Site by Transforming Oryza sativa Protoplast with Two Different Targeting RNP Complexes

1. The Oryza sativa protoplast preparation and PEG-mediated transformation were performed based on the partially modified method that had been published (Bart et al., 2006), and the specific preparation steps were as follows:
(1) The Oryza sativa seedlings for protoplasts were firstly prepared. The rice variety was Nipponbare. The rice seeds were hulled first, and the hulled seeds were rinsed with 75% ethanol for 1 minute, treated with 5% (v/v) sodium hypochlorite for 20 minutes, then washed with sterile water for more than 5 times, placed on an ultra-clean table and blow-dried, and then placed in a tissue culture flask containing ½ MS medium, with 20 seeds in each flask. Protoplasts were prepared by incubating the seeds at 26° C. and 12 hours of light for about 10 days.
(2) The leaf sheath of the seedlings was selected, cut with a sharp Gillette razor blade into about 1 mm pieces, and placed in 0.6M mannitol and MES culture medium (formulation: 0.6M mannitol, 0.4M MES, pH 5.7) for later use. After all the materials were cut, they were transferred to 20 mL of enzymolysis solution (formulation: 1.5% cellulase R10/RS (YaKult Honsha), 0.5% Mecerozyme R10 (YaKult Honsha), 0.5M mannitol, 20 mM KCl, 20 mM MES, pH5.7, 10 mM CaCl₂, 0.1% BSA, 5 mM β-mercaptoethanol), wrapped in aluminum foil and placed in a shaker at 28° C. The enzymolysis was carried out at 50 rpm in the dark for about 4 hours, and the speed was increased to 100 rpm for the last 2 minutes;
(3) After enzymolysis, an equal volume of W5 solution (formulation: 154 mM NaCl, 125 mM CaCl₂, 5 mM KCl, 15 mM MES) was added, shaken horizontally for 10 seconds to release the protoplast. The cells resulted from enzymolysis were filtered through a 300-mesh sieve and centrifuged at 150 g for 5 minutes to collect protoplast;
(4) The cells were rinsed twice with W5 solution, and the protoplast was collected by centrifugation at 150 g for 5 minutes;
(5) The protoplast was resuspended with an appropriate amount of MMG solution (formulation: 3.05 g/L MgCl₂, 1 g/L MES, 91.2 g/L mannitol), and the protoplast concentration was about 2×10⁶ cells/mL.

2. Preparation of RNP Complex:

According to the OsALS548 target site sequence GGGTATGGTTGTGCAATGGGAgga (the OsALS W548 site was underlined, corresponding to Arabidopsis thaliana ALS W574, and the PAM site recognized by Cas9 protein was shown in italic lowercase), the purified NGA-Cas9 protein was selected to prepare the RNP complex. The GGA was used as PAM to design >CrRNA1-548-G: 5′-GGGUAUGGUUGUGCAAUGGGAguuuuagagcuaugcu-3′, it was predicted that one G base would be deleted between the first 3-4 positions of PAM after editing, and then the sequence resulted from the above deletion was used to design a second >CrRNA2-548+T: 5′-UGGGUAUGGUUGUGCAAUGGAguuuuagagcuaugcu-3′, it was predicted that one T base would be inserted after the second editing to obtain the conversion of TGG-TTG.

>CrRNA1-548-G and >CrRNA1-548+T were synthesized by GenScript Biotechnology Company, and sgRNA was also synthesized:

>sgRNA1-548-G:
5′GGGUAUGGUUGUGCAAUGGGAguuuuagagcu
agaaauagcaaguuaaaauaaggcuaguccguua
ucaacuugaaaaaguggcaccgagucggugc3′
>sgRNA2-548+T:
5′UGGGUAUGGUUGUGCAAUGGAguuuuagagcu
agaaauagcaaguuaaaauaaggcuaguccguua
ucaacuugaaaaaguggcaccgagucggugc3′.

The synthesized crRNA and GenCRISPR tracrRNA (GenScript SC1933) were mixed equimolarly, added with crRNA&tracrRNA annealing buffer (GenScript SC1957-B) and annealed to prepare gRNA according to the instructions. The tracrRNA sequence was 5′-agcauagcaaguuaaaauaaggcuaguccguuaucaacuugaaaaaguggcaccgagucggugcuuu-3.

The RNP reaction system was prepared according to the following table, and after the reaction system was prepared, it was incubated at 25° C. for 10 minutes.

| Components | Volume | |
|---|---|---|
| NGA Cas9 protein | 10 | μL (20 μg) |
| gRNA or sgRNA | 10 | μL (20 μg) |
| 10x× Cas9 reaction buffer | 10 | μL |
| RNase-free ultrapure water | 70 | μL |

3. Protoplast Transformation
(1) 200 μl of MMG resuspended protoplast as prepared above was taken and added with the RNP complex (20 μg of Cas9 protein, 20 μg of sgRNA) generated after the incubation, and gently flicked to mixed well.
(2) An equal volume of 40% (w/v) PEG solution (formulation: 40% (w/v) PEG, 0.5M mannitol, 100 mM CaCl₂) was added, gently flicked to mix well, and stood at 28° C. in dark for 15 minutes;
(3) After induction and transformation, 1.5 mL of W5 solution was added slowly, gently flicked to mix the cells well, and the cells were collected by centrifugation at 150 g for 3 minutes. This step was repeated once;
(4) the cells were resuspended by adding 1.5 mL of W5 solution, placed in a 28° C. incubator and cultured in dark for 12-16 hours. The cells should be incubated for 48 to 60 hours if they were used to extract protoplast genomic DNA.

4. Detection of Genome Target Editing Event
(1) The protoplast DNA was extracted using the partially modified CTAB method, and the method was specifically described as follows: the protoplast was centrifuged and the supernatant was discarded, 500 μL of DNA extraction solution was added, shaken and mixed well, and incubated in a 65° C. water bath for 1 hour; the sample was cooled after the incubation in water bath, added with an equal volume of chloroform, inverted for several times to mix well and centrifuged at 10,000 rpm for 10 minutes; 400 μl of supernatant was taken and transferred to a new 1.5 mL centrifuge tube, added with 1 mL of 70% (v/v) ethanol, and stood to precipitate at −20° C. for 20 minutes; the DNA was precipitated by centrifugation at 12,000 rpm for 15 minutes, the precipitate was dried in the air and then dissolved by adding 50 μl of ultrapure water, and stored at −20° C. for later use.
(2) The gene-specific primers were used to amplify the fragment containing the W548 target site, and the following detection primers were designed for the target site:

OsALS500AA-F:
5′GGCTAACCCAGGTGTCACAG3′,
OsALS-3′UTR-R:
5′CCATGCCAAGCACATCAAACAAG3′

The PCR reaction system was shown in the following table:

| Components | Volume |
|---|---|
| 2× I5 reaction solution | 25 μL |
| Forward primer (10 uM) | 2 μL |
| Reverse primer (10 uM) | 2 μL |
| Genomic DNA template | 2 μL |
| Ultrapure water | Added to 50 μL |

(3) The PCR reaction was established, and the general reaction conditions were shown in the following table:

| Step | Temperature | Time | |
|---|---|---|---|
| Pre-denaturation | 98° C. | 30 | s |
| 30-35 amplification cycles | 98° C. | 15 | s |
| | 58° C. | 15 | s |
| | 72° C. | 30 | s |
| Final extension | 72° C. | 3 | min |

(4) The detection was carried out by agarose gel electrophoresis, and the PCR fragments were recovered and sequenced.

5. Experimental Results:

By either using the gRNA prepared with the synthetic crRNA and tracrRNA by annealing or directly using the synthetic sgRNA, an active RNP complex could be formed with the purified NGA-Cas9 protein. By sequencing the OsALS548 targeted site, the mutation from TGG to TTG could be detected, which demonstrated that the site-specific mutation of the target site in the cell could be achieved by the programmed sequential cutting/editing generated from the RNP complex in combination with crRNA or sgRNA in sequential order. As shown in FIG. 14, at the arrow point in the diagram of protoplast OsALS548 target sequencing peaks, in addition to the original G base signal peak, there was also the T base signal peak generated from the mutation, which resulted in the W548L mutation at OsALS548 site.

### Example 9: Achieving W2038L Mutation at OsACCase2 W2038 Site by Bombarding Oryza sativa Callus with Two Different Targeted RNP Complexes

1. Preparation of RNP Complexes:

According to the OsACCase2 W2038 target site sequence GTTCATCCTCGCTAACTGGAGagg, in which the OsACCase2 W2038 site, corresponding to the Alopecurus myosuroides ACCase W2027, was underlined, and the PAM site recognized by the Cas9 protein was shown in italic lowercase, the purified SpCas9 protein was selected to prepare the RNP complexes. The GGA was used as PAM to design >CrRNA1-2038-G: 5′-GUUCAUCCUCGCUAACUGGAGguuuuagagcuaugcu-3′, it was predicted that one G base would be deleted between the first 3-4 positions of PAM after editing, then the sequence generated from the deletion was used to design a second >CrRNA2-2038+T: 5′-UGUUCAUCCUCGCUAACUGAGguuuuagagcuaugcu-3′, it was predicted that one T base would be inserted after the second editing, thus the conversion of TGG-TTG was obtained.

>CrRNA1-2038-G and >CrRNA2-2038+T were synthesized by GenScript Biotechnology Company, and sgRNA was also synthesized:

>sgRNA1-2038−G:
5′GUUCAUCCUCGCUAACUGGAGguuuuagagcu
agaaauagcaaguuaaaauaaggcuaguccguua
ucaacuugaaaaaguggcaccgagucggugc 3′
>sgRNA2-2038+T:
5′UGUUCAUCCUCGCUAACUGAGguuuuagagcu
agaaauagcaaguuaaaauaaggcuaguccguua
ucaacuugaaaaaguggcaccgagucggugc 3′.

The synthesized crRNA and GenCRISPR tracrRNA (GenScript SC1933) were mixed equimolarly, added with crRNA&tracrRNA annealing buffer (GenScript SC1957-B) and annealed to prepare gRNA according to the instructions.

The RNP complexes were prepared by incubating in the same reaction system as in Example 8. Taking the amount of 10 gene gun bombardments for transformation as example: 20 μg of Cas9 protein, 20 μg of gRNA or sgRNA, 10 μl of 10×Cas9 reaction buffer, made up to 100 μl in total with RNase-free ultrapure water, incubated at 25° C. for 10 minutes, and mixed gently.

2. Induction of Oryza sativa Callus

The mature and plump Oryza sativa seeds were selected, hulled, and disinfected according to the following steps:
(1) The rice (Oryza sativa) seeds were hulled, wherein the rice variety was Huaidao No. 5, purchased from the seed market.
(2) The seeds were washed with sterile water for unlimited times until the washed water became clear.
(3) After being sterilized with 70% alcohol for 1 minute, the seeds were then placed in 10% sodium hypochlorite on a horizontal shaker and incubated for 25 minutes under shaking.
(4) After sodium hypochlorite disinfection, the seeds were washed with sterile water for 5 times. The seeds were seeded on callus induction medium (formulation: MS powder (4.42 g/L)+2,4-D (2 mg/L)+sucrose (30 g/L)+phytagel (4 g/L)), and cultivated in the dark at 28° C. to induce callus.

3. Gene Gun RNP Bombardment:
(1) Hypertonic cultivation

The callus with good embryogenicity was transferred to hypertonic medium (formulation: MS powder (4.42 g/L)+2,4-D (2 mg/L)+sucrose (30 g/L)+D-mannitol (0.4M))+phytagel (4 g/L)), sterile operation was carried out on an ultra-clean bench, and cultivation was carried out in the dark at 25° C. for 4-6 hours.
(2) Preparation of gold powder suspension: by using an imported 1.5 mL EP tube, 30 mg of gold powder (diameter 0.6 μm) was weighed; added with 1 mL of 70% ethanol, subjected to vortex well, and stood on ice for 10 minutes. After centrifugation for 1 minute, the supernatant was discarded; 1 mL of sterile water was added, subjected to vortex thoroughly, centrifuged for 1 minute, the supernatant was discarded, and the above operation was repeated for 3 times. 500 μL of sterilized glycerol (50%) was added, subjected to vortex thoroughly to prepare a gold powder suspension with a concentration of 60 μg/μl, which was stored at −20° C.
(3) 50 μl of gold powder suspension (60 μg/ml) was taken, added with 100 μL of the prepared RNP complex, and mixed gently.
(4) 15 μl of RNP/gold powder mixture was taken and placed in the center of a cleavable membrane of PDS-1000 benchtop gene gun (Bio-Rad), blow-dried, and then bombarded according to the instructions of the instrument.

Bombardment parameters: the vacuum degree was 26-28, the distance was 6 cm, and the air pressure was 1100 psi or 1350 psi.
(5) After the bombardment was completed, the callus was cultured on a hypertonic medium at 25° C. overnight (for 16 hours) in the dark.

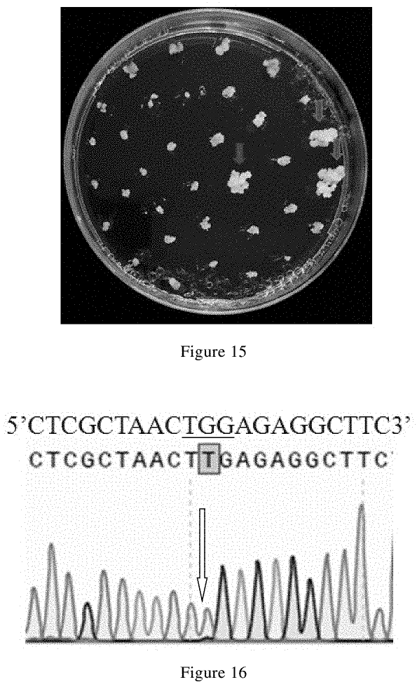

4. Selection, Differentiation and Rooting:
(1) After the bombarded callus was cultured overnight, it was transferred to the induction medium, and cultured to recover at 28° C. for one week.
(2) After one week of recovery, the callus was transferred to a selection medium (formulation: 4.1 g/L N6 powder+0.3 g/L hydrolyzed casein+2.8 g/L proline+2 mg/L 2,4-D+3% sucrose+50 ug/L quizalofop-p+500 mg/L Cef (cephalosporin)+0.1 g/L inositol+0.35% phytagel, pH5.8) to perform selection for 3-4 weeks; for the prospective editing of OsACCase2 W2038, 50 ug/L quizalofop-p was used for selection, as shown in FIG. 15.
(3) The selected resistant callus with good growth status was transferred to a differentiation medium (formulation: MS powder (4.42 g/L)+KT (1 mg/L)+sucrose (30 g/L)+phytagel (4.5 g/L)+50 ug/L quizalofop-p, pH 5.8), and cultured at 28° C. under light to induce differentiation for 2-4 weeks.
(4) The differentiated seedlings were transferred to a rooting medium (formulation: ½ MS powder (2.3 g/L)+sucrose (30 g/L)+phytagel (4.5 g/L)) for rooting, the seedlings were tempered after the rooting was completed, and then transferred to pots filled with soil and placed in a greenhouse for cultivation.

5. Detection of Target Editing Events in Resistant Callus and TO Tissue Culture Seedlings:

A total of 11 resistant calli were obtained in the selection, the DNA thereof was extracted by the CTAB method. The detection primers for the target site were designed as follows: OsACC2038test-F: 5′CTGTAGGCATTTGAAACTGCAGTG3′, OsACC2038test-R: 5′GCAATCCTGGAGTTCCT-CTGACC3′, and the PCR fragment containing OsACCase2 W2038 site was amplified, recovered and sequenced. The sequencing detection indicated that 10 out of them had the mutation from TGG to TTG at the OsACCase2 W2038 site, in which 3 samples were homozygous mutants.

For the T0 generation tissue culture seedlings obtained by resistant callus differentiation, the DNA thereof was extracted for detecting the editing target site sequence, and there was also a homozygous mutation from TGG to TTG at the OsACCase2 W2038 site, as shown in FIG. 16.

6. Resistance Test of T1 Generation Seedlings to ACCase Inhibitor Herbicides

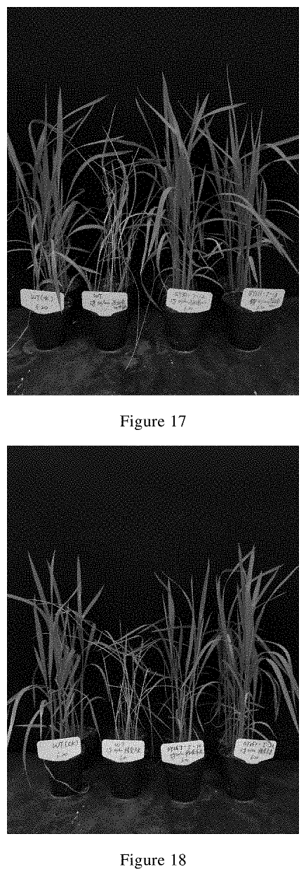

After the propagation of the TO strain containing the W2038L mutation, the T1 generation mutant seedlings were tested for herbicide resistance with quizalofop-p and haloxyfop-p in field concentrations. It could be seen that the OsACCase2 W2038L mutant strain was significantly resistant to these two ACCase inhibitor herbicides, as shown in FIGS. 17-18.

In summary, by either using the gRNA prepared with the synthetic crRNA and tracrRNA by annealing, or directly using the synthetic sgRNA, an active RNP complex could be formed with the purified SpCas9 protein; the gene gun bombarded calli could be selected by using quizalofop-p in the tissue culture stage; the homozygous mutation from TGG to TTG could be detected by sequencing the OsACCase2 W2038 target site of the TO generation tissue culture seedlings; the mutation could be inherited to the T1 generation and showed resistance to ACCase inhibitor herbicides, which further demonstrated that the programmed sequential cutting/editing generated from the RNP complex in combination with the sequential targeting crRNA or sgRNA could achieve site-specific mutation of a target site in the cell, and could guide the production of cell-endogenous selection markers for tissue culture selection, thereby creating herbicide-resistant crops.

### Example 10: Different Targeting RNP Complexes Simultaneously Edit OsACCase2 W2038 Site and OsBADH2 Gene of Oryza sativa Callus

The RNP complex was prepared by the method according to Example 8, the gene gun bombardment and tissue culture procedures were the same as those of Example 9. Besides the crRNA or sgRNA targeting the OsACCase2 W2038 site, the crRNA or sgRNA targeting the OsBADH2 gene was added at the same time, and they were incubated with SpCas9 protein to form a targeting RNP complex for a second target gene OsBADH2. The gene gun bombardment was performed, the TO generation tissue culture seedlings were obtained after recovery culture, screening, differentiation and rooting, and the T1 generation was obtained by propagation.

The Oryza sativa OsBADH2 genome sequence was shown in SEQ ID NO: 17. According to the CRISPOR online tool (http://crispor.tefor.net/), the target site sequence CCAAGTACCTCCGCGCAATCGcgg was selected, in which the PAM site recognized by the Cas9 protein was shown in italic lowercase, and the purified SpCas9 protein was selected to prepare an RNP complex. The CGG was used as PAM to design >CrRNA1-OsBADH2: 5′-CCAAGUACCUCCGCGCAAUCGguuuuagagcuaugcu-3′, it was predicted that the resistant mutation of OsACCase2 W2038L and the knockout mutation event of OsBADH2 could be simultaneously detected in the resistant callus obtained by the selection of quizalofop-p.

>CrRNA1-OsBADH2 was synthesized by GenScript Biotechnology Company, and sgRNA was also synthesized:

>OsBADH2-sgRNA:
5′CCAAGUACCUCCGCGCAAUCGguuuuagagc
uagaaauagcaaguuaaaauaaggcuaguccgu
uaucaacuugaaaaaguggcaccgagucggug
c3′.

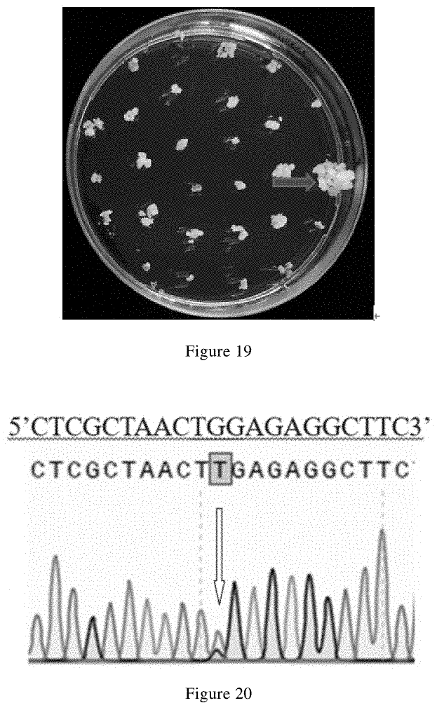

The resistant callus was selected according to the transformation steps in Example 9, as shown in FIG. 19, and the resistant callus was selected and subjected to differentiation and seedling emergence, and the sequences at the OsACCase2 and OsBADH2 target sites of the callus and TO generation tissue culture seedlings were sequenced. The OsBADH2 target detection primers were:

OsBADH2-check F:
5′CATCGGTACCCTCCTCTTC3′
OsBADH2-check R:
5′ATCGATCGATTTGGGGCTCA3′

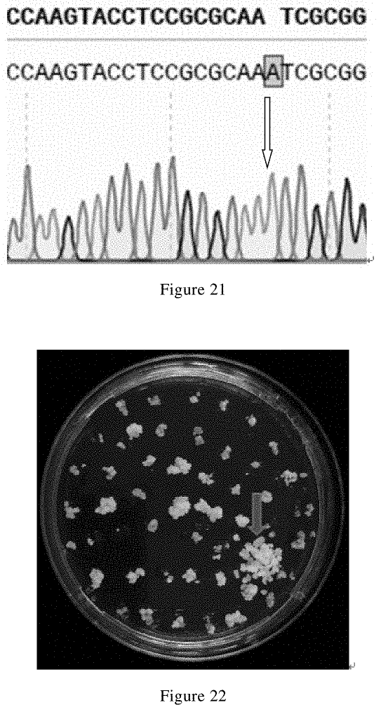

As a result, a total of 13 resistant calli were obtained by selection, the OsACCase2 W2038L mutation event was detected in 11 out of them, and the detection of OsBADH2 target sequence for these 11 callus samples showed that 8 out of them simultaneously contained the editing event at OsBADH2 target site. In the TO generation tissue culture seedlings obtained by differentiation, the existence of OsACCase2 W2038L mutation and OsBADH2+A homozygous mutation was detected, as shown in FIGS. 20-21.

In sum, after the rice callus was subjected to gene gun bombardment with the sequential targeting crRNA- or sgRNA-targeting RNP complex to perform site-specific editing of OsACCase2 W2038 and simultaneously adding the targeting RNP complex targeting the second target gene OsBADH2, the selection of the callus could be performed in the tissue culture stage with quizalofop-p; the OsACCase2 W2038 mutation and the targeted knockout of OsBADH2 simultaneously occurred in 61% of the resistant callus, and the strains containing homozygous mutation of OsBADH2 were detected in the TO generation tissue culture seedlings. These indicated that the sequential targeting in combination with the RNP transformation for site-specific editing of resistance genes could generate endogenous selection markers, and the addition of the corresponding selection pressure could simultaneously screen the editing events of the second target gene, thereby achieving the site-specific editing of genome by non-transgenic means.

### Example 11: Different Targeted RNP Complexes Simultaneously Edit OsALS548 Site and OsSWEET14 Gene of Oryza sativa Callus

The targeting RNP complexes for the OsALS548 site were prepared by the method according to Example 8, in which the crRNA and sgRNA sequences were the same as in Example 8, respectively:

>CrRNA1-548−G:
5′-GGGUAUGGUUGUGCAAUGGGAguuuuagagcuaugcu-3′,
>CrRNA2-548+T:
5′-UGGGUAUGGUUGUGCAAUGGAguuuuagagcuaugcu-3′,
>sgRNA1-548−G:
5′GGGUAUGGUUGUGCAAUGGGAguuuuagagcu
agaaauagcaaguuaaaauaaggcuaguccguua
ucaacuugaaaaaguggcaccgagucggugc3′,
>sgRNA2-548+T:
5′UGGGUAUGGUUGUGCAAUGGAguuuuagagcu
agaaauagcaaguuaaaauaaggcuaguccguua
ucaacuugaaaaaguggcaccgagucggugc3′.

Referring to Example 8, the gRNA or sgRNA was incubated with NGA-Cas9 to prepare RNP complexes targeting OsALS548 site.

In addition to the targeted RNP complex for the OsALS548 site, the crRNA or sgRNA for the OsSWEET14 gene was added at the same time, and the targeting RNP complex for the second target gene OsSWEET14 was formed by incubation with the SpCas9 protein. The TO generation tissue culture seedlings were obtained by performing gene gun bombardment, recovery culture, selection, differentiation, rooting, and the T1 generation was obtained by propagation. The selection pressure was 5 mg/L pyroxsulam.

The Oryza sativa OsSWEET14 genome sequence was shown in SEQ ID NO: 18. According to the CRISPOR online tool (http://crispor.tefor.net/), the target site sequence GAGCTTAGCACCTGGTTGGAGggg was selected, in which the PAM sites recognized by the SpCas9 protein was shown in italic lowercase, and the purified SpCas9 protein was selected to prepare the RNP complex. The GGG was used as PAM to design:

>CrRNA1-OsSWEET14:

5′-GAGCUUAGCACCUGGUUGGAGguuuuagagcuaugcu-3′, and it was predicted that the resistant mutation of OsALS W548L and the knockout mutation of OsSWEET14 could be simultaneously detected in the resistant callus obtained by the selection of pyroxsulam.

>CrRNA1-Os SWEET14 was synthesized by GenScript Biotechnology Company, and the sgRNA was also synthesized:

>Os SWEET14-sgRNA:
5′GAGCUUAGCACCUGGUUGGAGguuuuagagcu
agaaauagcaaguuaaaauaaggcuaguccguua
ucaacuugaaaaaguggcaccgagucggugc3′.

The resistant callus was selected according to the gene gun bombardment and tissue culture procedures as described in Example 9, as shown in FIG. 22, and the resistant callus was selected to perform differentiation and seedling emergence, in which the selection medium formulation was: 4.1 g/L N6 powder+0.3 g/L hydrolyzed casein+2.8 g/L proline+2 mg/L 2,4-D+3% sucrose+5 mg/L pyroxsulam+500 mg/L Cef (cephalosporin)+0.1 g/L inositol+0.35% phytagel, pH 5.8.

The sequences at the OsALS548 site and OsSWEET14 target site of the callus and TO generation tissue culture seedlings were sequenced. The OsSWEET14 target site detection primers were:

OsSWEET14-check F:
5′ ATGGGTGCTGATGATTATCTTGTAT3′
OsSWEET14-check R:
5′ TGAAGAGACATGCCAGCCATTG3′

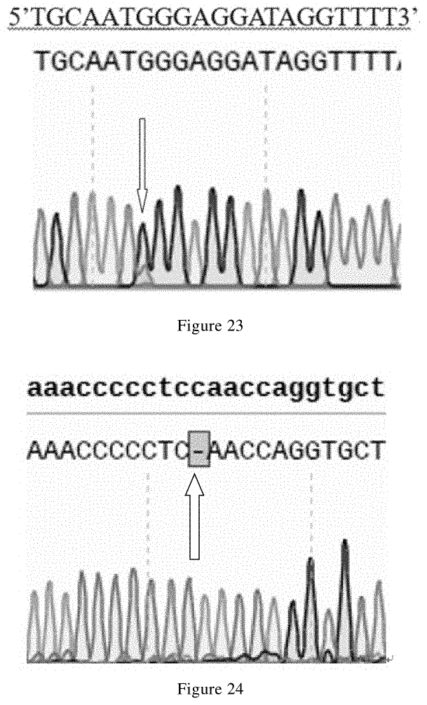

As a result, a total of 9 resistant calli were obtained by the selection, the OsALS W548L mutation event was detected in 8 out of them, and the detection of OsSWEET14 target sequence in these 8 callus samples showed that 5 out of them also contained the editing event at OsSWEET14 target site. In the TO generation tissue culture seedlings obtained by differentiation, both the OsALS W548L mutation and the OsSWEET14-C homozygous mutation could be detected, as shown in FIGS. 23-24.

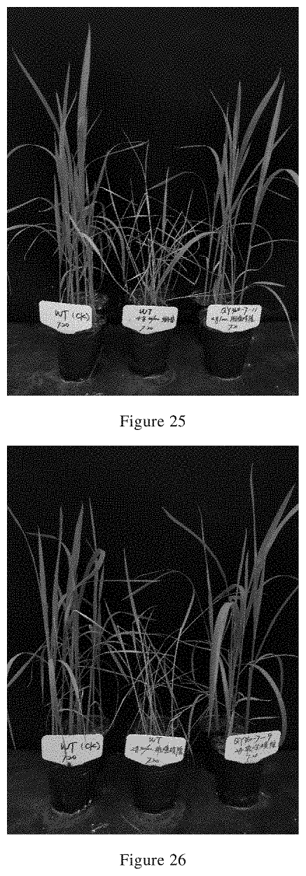

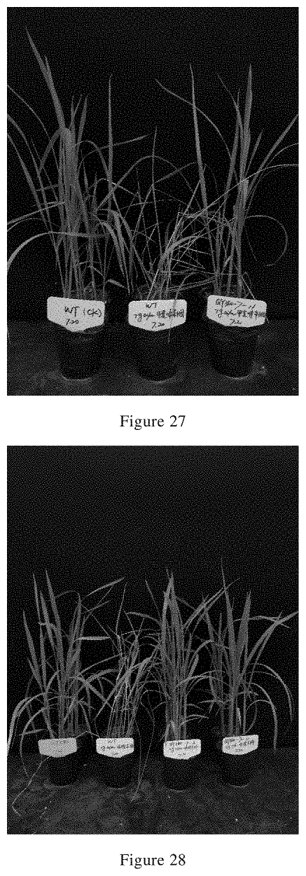

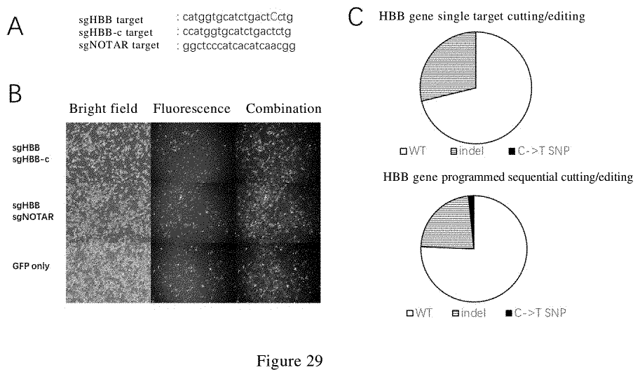

After the propagation of TO strains in which the occurrence of OsALS W548L mutation was detected, the T1 generation mutant seedling strains were tested for herbicide resistance with pyroxsulam, imazapic, nicosulfuron and flucarbazone-Na at field concentrations. It could be seen that the OsALS W548L mutant strain showed significant resistance to all of these 4 ALS inhibitor herbicides, as shown in FIGS. 25-28.

In summary, by either using the gRNA prepared with the synthetic crRNA and tracrRNA by annealing or directly using the synthetic sgRNA, an active RNP complex could be formed with the purified NGA Cas9 protein, the site-specific mutation of a target site in the cells could be achieved by the programmed sequential cutting/editing generated from the RNP complex in combination with the sequential targeting crRNA or sgRNA, the selection of the callus bombarded by gene gun could be performed with pyroxsulam at the tissue culture stage, the mutation from TGG to TTG was detected by sequencing the OsALS548 target site of the TO generation tissue culture seedlings, and this mutation could be inherited to the T1 generation and showed resistance to ALS inhibitor herbicides.

After the rice callus was subjected to gene gun bombardment by simultaneously adding the targeting RNP complex targeting the second target gene OsSWEET14 and using the SpCas9 protein that recognized NGG PAM, the selection of the callus was performed with pyroxsulam at the tissue culture stage. The OsALS W548L mutation and the targeted knockout of OsSWEET14 occurred simultaneously in 55% of the resistant callus, the occurrence of homozygous mutation of OsSWEET14 could be detected in the TO generation tissue culture seedlings, which further indicated that the endogenous selection markers could be generated by the programmed sequential cutting/editing in combination with the site-specific editing of resistant genes generated from PNP transformation, and the editing events of the second target gene could be selected at the same time by simultaneously using the Cas9 proteins that recognized different PAM sites and adding a corresponding selection pressure, thereby achieving the site-specific editing of genome by non-transgenic means.

### Example 12: Generation of W561L Mutation at StALS561 Site by Bombarding Potato (Solanum tuberosum L.) Explants with Two Different Targeted RNP Complexes

The amino acid sequence of Solanum tuberosum L. StALS2 protein was shown in SEQ ID NO: 19, and the sequence of Solanum tuberosum L. StALS2 gene was shown in SEQ ID NO: 20. The methods for the preparation of RNP complexes and the gene gun bombardment referred to Examples 8-9, and the sgRNAs for the original sequence and the edited sequence designed for StALS2W561 site of Solanum tuberosum L. StALS2 corresponding to the Arabidopsis thaliana ALS574 site were as follows:

>StALS561−G:
5′GGGAAUGGUGGUUCAGUGGGAguuuuagagcu
agaaauagcaaguuaaaauaaggcuaguccguua
ucaacuugaaaaaguggcaccgagucggugc3′
>StALS561+T:
5′UGGGAAUGGUGGUUCAGUGGAguuuuagagcu
agaaauagcaaguuaaaauaaggcuaguccguua
ucaacuugaaaaaguggcaccgagucggugc3′,

And the RNP complexes were prepared according to the method described in Example 8.

The recipient potato variety was Atlantic or Favorita, and the leaves, stems and axillary buds thereof were used as explants, respectively. The methods for gene gun bombardment and selection and differentiation were as follows:
(1) Leaves: The whole leaf was cut and spread flat on the hypertonic M6 medium (formulation: 4.42 g/L MS powder+1 ml/L B5 vitamins (Phytotechlab, G219)+30 g/L sucrose+8 g/L agar+2 mg/L 2,4-D+0.8 mg/L zeatin riboside+0.2M mannitol+250 mg/L Cef), about 5-6 leaves were used for each shot of bombardment. After pre-incubation for 24 hours, the bombardment with gold powder was performed. After bombardment, they were continuously cultured in hypertonic M6 medium in dark for 2 days, then transferred to M6 medium (4.42 g/L MS powder+1 ml/L B5 vitamins (Phytotechlab, G219)+30 g/L sucrose+8 g/L agar+2 mg/L 2,4-D+0.8 mg/L zeatin riboside+250 mg/L Cef) and continuously cultured for 1 week, then transferred to M6 medium with selection pressure after 1 week, the selection of resistant callus was performed with the selection pressure of 20 μg/L chlorsulfuron (formulation: 4.42 g/L MS powder+1 ml/L B5 vitamins (Phytotechlab, G219)+30 g/L sucrose+8 g/L agar+2 mg/L 2,4-D+0.8 mg/L zeatin riboside+250 mg/L Cef+20 μg/L chlorsulfuron), 4 weeks later, they were transferred to an bud induction medium R4 containing selection pressure (formulation: 4.42 g/L MS powder+1 ml/L B5 vitamins (Phytotechlab, G219)+30 g/L sucrose+8 g/L agar+2 mg/L GA3+0.8 mg/L zeatin riboside+250 mg/L Cef+20 μg/L chlorsulfuron) until seedling emergence.
(2) Stems: The potato stems (without axillary buds) were taken and cut longitudinally with the incisions facing upwards, and placed on CIMI medium (4.42 g/L MS powder+20 g/L sucrose +8 g/L agar+0.5 mg/L zeatin nucleoside+2 mg/L 2,4-D+250 mg/L Cef+20 μg/L chlorsulfuron), followed by gene gun bombardment, cultured in the dark for 1 day after bombardment, the stems were transferred to fresh CIMI medium and continuously cultured for 1 week, then the stems were transferred to SIMI medium containing selection pressure (formulation: 4.42 g/L MS powder+20 g/L sucrose+8 g/L agar+1 mg/L zeatin riboside+0.1 mg/L GA3+250 mg/L Cef+20 μg/L chlorsulfuron) to perform the selection for resistant buds until seedling emergence.
(3) Axillary buds: The axillary buds were taken from potato stems, cut longitudinally, and transferred to CIMI medium, with the incisions facing upwards, placed on CIMI medium, followed by gene gun bombardment, cultured in the dark for 1 day after bombardment, they were transferred to fresh CIMI medium and continuously cultured for 1 week, and then they were transferred to SIMI medium containing selection pressure to perform the selection for resistant buds until seedling emergence.

The detection primers for StALS2 W561 site were:

StALS561-Check F:
5′GTGGATTAGGAGCAATGGGATTT3′
StALS561-Check R:
5′ TTATTTTAGATAATACAATGCCTCG3′

After detection, the editing event of W561L occurred at the StALS2 W561 site of the resistance-screened TO generation potato tissue culture seedlings, which demonstrated that the non-transgenic transient gene editing method provided by the present invention was suitable for crops such as potatoes from which an exogenous transgenic element can hardly be separated and removed by selfing or hybridization.

### Example 13: Successful Use of Programmed Sequential Cutting/Editing Scheme for Base Substitution in Human 293T Cells

The HBB (hemoglobin subunit beta) gene in human embryonic kidney cell 293T (the DNA sequence thereof was shown in SEQ ID NO: 21, the CDS sequence thereof was shown in SEQ ID NO: 22, and the amino acid sequence thereof was shown in SEQ ID NO: 23) was selected, the target site for sequential targeting of sgRNA was designed in the region of the first exon, in which the first target was catggtgcaCctgactcctgAGG. The sgRNA that recognized this target was named sgHBB, and it was predicted that the deletion of one C base could be generated at the sgRNA cut of this site. The second target was ccatggtgcatctgactctgAGG, which recognized the sequence with the deletion of one C base generated from the cutting/editing of the first target, and the sgRNA of this target was named sgHBB-c. The sgRNA with no target site in 293T cells was designed and named sgNOTAR, which was used as a complementing plasmid for transfection in the experiment.

According to the above design, complementary single-stranded DNA fragments were synthesized respectively. After annealing, they were ligated into px458 (addgene: 48138) plasmids digested with BbsI enzyme, and transformed to E. coli DH5a competent. After the resultant E. coli single colonies were verified by sequencing, the plasmids were extracted and purified with an endotoxin-free plasmid extraction kit (Tiangen Bio).

The vigorously growing 293T cells were digested and isolated with 0.05% trypsin (Gibico), diluted with DMEM medium (10% fetal bovine serum; penicillin+streptomycin double resistant) and inoculated into 24-well culture plates, and placed in a carbon dioxide incubator overnight. On the next day, they were mixed separately with, according to sequential cutting/editing: sgHBB and sgHBB-c plasmids each 0.5 ug; single target cutting/editing: sgHBB and sgNOTAR plasmids each 0.5 ug; no target control: pEGFP-c1 plasmid 1 μg. The transformation was performed with lipofectamine3000 (Invitrogen). There were 3 duplications for each group.

48 hours After transformation, pictures were taken with a fluorescence microscope to record the transformation efficiency, and the total DNA of each well was extracted with a nucleic acid extraction kit (Omega).

The designed Hi-tom sequencing primers were as follows:

Hi-HBB-F:
gaggtgagtacggtgtgcGCTTACAT
TTGCTTCTGACACAACT;
Hi-HBB-R:
gagttggatgctggatggTCTATTGG
TCTCCTTAAACCTGTCTTG.

These primers were used to perform PCR for each DNA sample. The high-throughput sequencing of PCR products was performed by using Hi-tom method (Sci China Life Sci. 2019 January; 62(1):1-7. doi: 10.1007/s11427-018-9402-9).

The statistical data of the sequencing results were shown in Table 6 and Table 7. These data indicated that the sequential cutting/editing method at HBB site produced an editing outcome from C to T (resulting in P6S mutation) at a ratio of about 1.67%, while the single target cutting/editing method could not produce such base substitution.

TABLE 6
Editing types produced by different sgRNA combinations in the sequential
cutting/editing experiment of HBB gene in 293T cells, and the readings of
different genotypes detected by Hi-Tom sequencing

| | HBB | HBB | HBB | | | | | |
|---|---|---|---|---|---|---|---|---|
| | programmed | programmed | programmed | HBB | HBB | HBB | | |
| | sequential | sequential | sequential | single target | single target | single target | GFP | GFP |
| | cutting/editing | cutting/editing | cutting/editing | cutting/editing | cutting/editing | cutting/editing | transformation | transformation |
| Genotype | repeat 1 | repeat 2 | repeat 3 | repeat 1 | repeat 2 | repeat 3 | repeat 1 | repeat 2 |
| WT | 6025 | 6037 | 6003 | 5776 | 5636 | 5614 | 7564 | 7430 |
| 1Deletion | 555 | 593 | 613 | 1170 | 1134 | 1120 | 252 | 288 |
| 2Deletion | 594 | 625 | 410 | 465 | 509 | 405 | | |
| 3Deletion | 304 | 263 | 258 | | 260 | 243 | | |
| 4Deletion | | 222 | | | | | | |
| 5Deletion | | | 220 | | 217 | | | |
| 6Deletion | | | | | | | | |
| 7Deletion | 312 | 223 | 232 | 274 | 229 | 278 | | |
| Insertion | | | | 273 | | 308 | | |
| C -> T SNP | 168 | | 230 | | | | | |
| total | 7958 | 7963 | 7966 | 7958 | 7985 | 7968 | 7816 | 7718 |

TABLE 7
Combined statistics of ratios of editing types generated by different sgRNA
combinations in the sequential cutting/editing experiment of HBB genes in 293T cells

| | | | | HBB | HBB | |
|---|---|---|---|---|---|---|
| | HBB | HBB | | programmed | single | GFP |
| | programmed | single | GFP | sequential | target | transformation |
| | sequential | target | transformation | cutting/editing | utting/editing | background |
| Genotype | cutting/editing | utting/editing | background | ratio | ratio | ratio |
| WT | 18065 | 17026 | 14994 | 75.63% | 71.21% | 96.52% |
| 1Deletion | 1761 | 3424 | 540 | 7.37% | 14.32% | 3.48% |
| 2Deletion | 1629 | 1379 | 0 | 6.82% | 5.77% | 0.00% |
| 3Deletion | 825 | 503 | 0 | 3.45% | 2.10% | 0.00% |
| 4Deletion | 222 | 0 | 0 | 0.93% | 0.00% | 0.00% |
| 5Deletion | 220 | 217 | 0 | 0.92% | 0.91% | 0.00% |
| 6Deletion | 0 | 0 | 0 | 0.00% | 0.00% | 0.00% |
| 7Deletion | 767 | 781 | 0 | 3.21% | 3.27% | 0.00% |
| Insertion | 0 | 581 | 0 | 0.00% | 2.43% | 0.00% |
| C -> T SNP | 398 | 0 | 0 | 1.67% | 0.00% | 0.00% |
| total | 23887 | 23911 | 15534 | 100.00% | 100.00% | 100.00% |

Note:
WT represents wild type;
Deletion represents genotype of deletion;
Insertion represents genotype of insertion;
C -> T SNP represents genotype with base substitution from C to T at the cut;
total represents grand total.

In addition, sickle cell anemia and β-thalassemia are both inherited anemias caused by mutations in the HBB gene encoding hemoglobin β subunit of adult. Patients with these diseases may need blood transfusion or other therapies throughout their lives. The results of the above experiment demonstrated that through the technical solution of programmed sequential cutting/editing as provided by the present invention, a combination of crRNA or sgRNA could be designed for the mutation site of the HBB gene to induce the expected repair at the mutation site, so that the cell produced active hemoglobin and restored function, thereby achieving therapeutic effect. That is, the composition provided by the present invention has a use in treatment of diseases.

After a variety of tests at the same time, because this novel method is completely based on the existing functions of Cas9, the method of the present invention will be fully applicable to achieve the new functions of base substitution, deletion and insertion of specific fragments in other organisms (plants, animals, fungi or bacteria, etc.) where Cas9 can work well.

All publications and patent applications mentioned in the description are incorporated herein by reference, as if each publication or patent application is individually and specifically incorporated herein by reference.

Although the aforementioned invention has been described in more details by way of examples and embodiments for clear understanding, it is obvious that certain changes and modifications can be implemented within the scope of the appended claims, and such changes and modifications are all within the scope of the present invention.

1.-102. (canceled)

103. A method for generating a new mutation in anorganism, comprising: sequentially generating two or more DNA breaks at a specific site in a genome of the organism and spontaneously repairing them respectively, wherein a later DNA break is generated based on a new sequence generated from a previous DNA break repair.

104. The method of claim 103, wherein the DNA break is achieved by delivering a nuclease with targeting property into a cell of the organism to contact with the specific site of genomic DNA.

105. The method of claim 104, wherein the nuclease with targeting property is a ZFN, TALEN or CRISPR/Cas system.

106. The method of claim 103, wherein the sequentially generating two or more DNA breaks at a specific site is that based on a new sequence formed by a previous DNA break repair event generated by ZFN or TALEN editing, a new ZFNor TALEN proteinis designed to cut the site again.

107. The method of claim 103, wherein the sequentially generating two or more DNA breaks at a specific site is that based on a new sequence formed by a previous DNA break repair event generated by a CRISPR/Cas system, a new target RNA is designed to cut the site again.

108. The method of claim 103, wherein the two or more DNA breaks are generated by sequentially delivering different targeted nucleases into recipient cells of different generations, wherein a mutant cell that has completed the previous editing is used as a recipient to receive the delivery of the targeted nuclease for the later editing, there by performing a second editing to generate a site-specific mutation.

109. The method of claim 103, wherein the two or more DNA breaks are generated by delivering different targeted nucleases for different targets into a same recipient cell.

110. The method of claim 103, wherein the two or more DNA breaks are generated when RNP complexes formed by a same CRISPR/Cas nuclease respectively with different gRNAs or sgRNAs sequentially cut corresponding target sequences; or when RNP complexes respectively formed by each of two or more CRISPR/Cas nucleases that recognize different PAM sequences with respective gRNA or sgRNA sequentially cut corresponding target sequences.

111. The method of claim 109, wherein the targeted nuclease is any CRISPR/Cas nuclease capable of performing a genomeediting.

112. The method of claim 108, wherein the targeted nuclease is in a form of DNA.

113. The method of claim 109, wherein the targeted nuclease is in a form selected from DNA, mRNA, and protein.

114. The method of claim 104, wherein the method for delivering a nuclease with targeting property into a cell is selected from: 1) a PEG-mediated cell transfection method; 2) a liposome-mediated cell transfection method; 3) an electroporation transformation method; 4) a microinjection; 5) a gene gun bombardment; and 6) an Agrobacterium-mediated transformation method.

115. A new mutation, which is obtained by the method of claim 103.

116. A protein or biologically active fragment thereof, comprising the new mutation of claim 115.

117. A nucleic acid, comprising a nucleic acid sequence or complementary sequence thereof that encodes the protein or biologically active fragment thereof of claim 116.

118. A nucleic acid, comprising (a) a nucleotide sequence encoding a target RNA, wherein the target RNA comprises at least two target RNAs, in which a first target RNA targets a DNA to cause a break in the DNA, and a latter target RNA targets a sequence generated from a previous break repair event and generates a break again; and optionally (b) a nucleotide sequence encoding a Cas polypeptide.

119. A recombinant expression vector, comprising the nucleic acid of claim 118, and a promoter operably linked thereto.

120. An expression cassette, comprising the nucleic acid of claim 118.

121. A host cell, comprising the expression cassette of claim 120.

122. An organism, which is regenerated from the host cell of claim 121.

123. A method for lysing a target DNA, comprising contacting the target DNA with a complex, wherein the complex comprises:
(a) a Cas polypeptide; and
(b) at least two target RNAs, in which the first target RNA targets the DNA to cause a break in the DNA, and the latter target RNA targets a sequence generated from a previous break repair event and generates a break again.

124. The method of claim 123, wherein the contacting comprises introducing the following into a cell: (a) the Cas polypeptide or a polynucleotide encoding the Cas polypeptide, and (b) the target RNA or a DNA polynucleotide encoding the target RNA.

125. A composition, comprising:
(a) a Cas polypeptide, or a polynucleotide encoding the Cas polypeptide; and
(b) at least two target RNAs, or DNA polynucleotides encoding the target RNAs, wherein a first RNA targets a DNA to cause a break in the DNA, and a latter target RNA targets a sequence generated from a previous break repair event and generates a break again.

126. A kit, comprising:
(a) a Cas polypeptide, or a nucleic acid comprising a nucleotide sequence encoding the Cas polypeptide; and
(b) at least two target RNAs, or nucleic acids comprising nucleotide sequences encoding the target RNAs, wherein a first RNA targets a DNA to cause a break in the DNA, and the latter target RNA targets a sequence generated from a previous break repair event and generates a break again;
wherein (a) and (b) are in a same or separate containers.

127. The kit of claim 126, wherein the target RNA is a sgRNA or gRNA; and wherein the target RNAs are in a same or separate container(s).

128. A method for screening editing events independent of an exogenous transgenic marker, comprising:
1) sequentially generating two or more DNA breaks in sequence at a specific site of a first target gene of a recipient cell and spontaneously repairing them respectively, wherein a later DNA break is generated based on a new sequence generated from a repair of a previous DNA break;
2) generating certain editing events after the specific site of the first target gene is sequentially cut and repaired, which confers a mutantcell with a resistance to a certain selection pressure to produce a phenotypic selectable trait, applying a corresponding selection pressure to make a selection for the trait, and isolating a cell, tissue, organ, or complete organism that contains such editing events;
3) optionally, in addition to the first target gene, using a targeted nuclease for at least one second target gene to edit another target site at the same time, and enriching and screening an editing event of the second target gene synchronously through the screening of the selectable trait generated by mutations of the first target gene, and isolating a cell, tissue, organ or complete organism that simultaneously contains the editing events of the first target gene and of the at least one second target gene.

129. The method of claim 128, wherein the first target gene is a gene locus encoding at least one phenotypic selectable trait, wherein the at least one phenotypic selectable trait is a resistance/tolerance trait or a growth advantage trait.

130. The method of claim 128, wherein the specific site of a first target gene is a site at which a certain type of mutation is generated after sequential cuttings and repairs, which is capable of conferring the recipient cell with a resistance to a certain selection pressure to produce at least one phenotypic selectable resistance/tolerance trait or growth advantage trait; wherein the certain type of mutation comprises substitution of single base, substitution of a plurality of bases, or insertion or deletion of an unspecified number of bases, and the certain selection pressure is an environmental pressure or a pressure resulted from an added compound.

131. The method of claim 128, wherein the DNA break is achieved by delivering a nuclease with a targeting property into a cell of the organism to contact with the specific site of genomic DNA.

132. The method of claim 128, wherein the two or more DNA breaks are sequentially generated in sequence at a specific site is that based on a new sequence formed by a previous DNA break repair event generated by a CRISPR/Cas system, a new target RNA is designed to cut the site again.

133. The method of claim 128, wherein the two or more DNA breaks are generated when RNP complexes formed by a same CRISPR/Casnuclease respectively with different gRNAs or sgRNAs sequentially cut corresponding target sequences; or generated when RNP complexes respectively formed by two or more CRISPR/Cas nucleases that recognize different PAM sequences with respective gRNA or sgRNA, sequentially cut corresponding target sequences.

134. The method of claim 128, wherein the targeted nuclease for at least one second target gene and the CRISPR/Cas nuclease used for generating DNA break at a specific site of the first target gene are the same or different.

135. The method of claim 128, wherein the targeted nuclease is in a form of DNA, mRNA or protein.

136. A method for non-transgenic transient editing of anorganismgenome, comprising:
1) designing and synthesizing a combination of at least two crRNA fragments or a combination of at least two sgRNA fragments for a specific site of a first target gene of a recipient cell, wherein the crRNA combination combined with tracrRNA or the sgRNA combination alone is capable of guiding a corresponding Cas protein to sequentially generate two or more DNA breaks at the specific site in the first target gene of the recipient cell and to spontaneously repair them respectively, wherein a later DNA break is generated based on a new sequence generated from a previous DNA break repair;
2) mixing an appropriate amount of CRISPR/Cas protein or corresponding mRNA thereof with the combination of crRNA fragments and tracrRNA fragment or with the combination of sgRNA fragments alone as above designed and synthesized in advance that is capable of guiding a site-specific editing of the first target gene to generate endogenous selection markers, optionally, further adding at least one of artificially synthesized crRNA and tracrRNA fragments or artificially synthesized sgRNA fragments targeting a second, third or more target genes, and carrying out incubation in vitro to form an RNP complex;
3) delivering the resulting RNP complex into the recipient cell and contacting with the specific site of genomic DNA to achieve gene editing;
4) according to a phenotypic selectable trait generated by the site-specific editing of the first target gene by the RNP complex, applying a corresponding selection pressure to make a selection for the trait, and isolating a cell, tissue, organ or complete organism that contains the editing event, and optionally, isolating a cell, tissue, organ or complete organism that simultaneously contains the editing events of the first target gene and of the at least one of a second, third or more target genes.

137. The method of claim 136, wherein the first target gene is a gene locus encoding at least one phenotypic selectable trait, wherein the at least one phenotypic selectable trait is a resistance/tolerance trait or a growth advantage trait.

138. The method of claim 136, wherein the specific site of the first target gene is a site at which a certain type of mutation is generated after sequential cuttings and repairs, which is capable of conferring the recipient cell with a resistance to a certain selection pressure to produce at least one phenotypic selectable resistance/tolerance trait or growth advantage trait; and wherein the certain type of mutation comprises substitution of single base, substitution of a plurality of bases, or insertion or deletion of an unspecified number of bases, the certain selection pressure is an environmental pressure or a pressure resulted from an added compound.

139. The method of claim 136, wherein the feature “to sequentially generate two or more DNA breaks at a specific site” is that based on a new sequence formed by a previous DNA break repair event generated by a CRISPR/Cas system, a new target RNA is designed to cut the site again.

140. The method of claim 136, wherein the two or more DNA breaks are generated when RNP complexes formed by a same CRISPR/Casnuclease respectively with different gRNAs or sgRNAs sequentially cut corresponding target sequences; or generated when RNP complexes respectively formed by each of two or more CRISPR/Cas nucleases that recognize different PAM sequences with respective gRNA or sgRNA, sequentially cut corresponding target sequences.

141. The method of claim 136, where in the at least one of artificially synthesized crRNA and tracrRNA fragments or artificially synthesized sgRNA fragments targeting a second, third or more target genes and the crRNA or sgRNA targeting the first target gene use Cas proteins that recognize the same or different PAM sequences.

142. The method of claim 136, wherein the organism is a plant and the recipient cell is replaced by a recipient plant cell or tissue; wherein the recipient plant cell or tissue is any cell or tissue that can serve as a recipient for transient expression and can be regenerated into a complete plant through tissue culture.

143. The method of claim 142, wherein the first target gene is at least one endogenous gene that encodes at least one phenotypic selectable trait selected from herbicide resistance/tolerance, wherein the herbicide resistance/tolerance is selected from resistance/tolerance to EPSPS inhibitor, resistance/tolerance to glutamine synthesis inhibitor, resistance/tolerance to ALS or AHAS inhibitor, resistance/tolerance to ACCase inhibitor, resistance/tolerance to carotenoid biosynthesis inhibitor, resistance/tolerance to cellulose inhibitor, resistance/tolerance to lipid synthesis inhibitor, resistance/tolerance to long-chain fatty acid inhibitor, resistance/tolerance to microtubule assembly inhibitor, resistance/tolerance to photosystem I electron shunting agent, resistance/tolerance to photosystem II inhibitor, resistance/tolerance to PPO inhibitor, and resistance/tolerance to synthetic growth hormone.

144. The method of claim 143, wherein the first target gene is ALS, and the specific site of gene is selected from the sites A122, P197, R198, D204, A205, D376, R377, W574, S653, and G654 in an Arabidopsis AtALS protein amino acid sequence, and amino acid sites in an ALS protein of another plant, which correspond to the amino acid sites by using the AtALS amino acid sequence as reference standard; or the crRNA or sgRNA targets a target sequence comprising a sequence encoding an AtALS protein amino acid sequence site selected from A122, P197, R198, D204, A205, D376, R377, W574, S653, G654, and any combination thereof, and a target sequence comprising a sequence encoding an amino acid site in an ALS protein of another plant, which corresponds to the amino acid site, and any combination thereof, by using the AtALS amino acid sequence as reference standard; or
the first target gene is ACCase, and the specific site of gene is selected from sites I1781, E1874, N1878, W1999, W2027, I2041, D2078, C2088, and G2096 in an Alopecurus myosuroides AmACCase protein amino acid sequence, and amino acid sites in an ACCase protein of another monocotyledonous plant, which correspond to the amino acid sites by using the AmACCase amino acid sequence as reference standard; or the crRNA or sgRNA targets a target sequence comprising a sequence encoding an AmACCase amino acid sequence site selected from I1781, E1874, N1878, W1999, W2027, I2041, D2078, C2088, G2096, and any combination thereof, and a target sequence comprising a sequence encoding an amino acid site in an ACCase protein of another monocotyledonous plant, which corresponds to the amino acid sites, and any combination thereof, by using the AmACCase amino acid sequence as reference standard; or
the first target gene is HPPD, and the specific site of gene is selected from sites H141, L276, P277, N338, G342, R346, D370, P386, K418, and G419 in an Oryza sativa OsHPPD protein amino acid sequence, and amino acid sites in an HPPD protein of another plant, which correspond to the amino acid sites by using the OsHPPD amino acid sequence as reference standard; or the crRNA or sgRNA targets a target sequence comprising a sequence encoding an OsHPPD amino acid sequence site selected from H141, L276, P277, N338, G342, R346, D370, P386, K418, G419, and any combination thereof, and a target sequence comprising a sequence encoding an amino acid site in an HPPD protein of another plant, which corresponds to the amino acid site, and any combination thereof, by using the OsHPPD amino acid sequence as reference standard; or
the first target gene is PPO, and the specific site of gene is selected from sites S128, V217, S223, V364, K373, L423, Y425, and W470 in an Oryza sativa OsPPO1 protein amino acid sequence, and amino acid sites in a PPO protein of another plant, which correspond to the amino acid sites by using the amino acid sequence of OsPPO1 as reference standard; or the crRNA or sgRNA targets a target sequence comprising a sequence encoding an OsPPO1 amino acid sequence site selected from S128, V217, S223, V364, K373, L423, Y425, W470, and any combination thereof, and a target sequence comprising a sequence encoding an amino acid site in a PPO protein of another plant, which corresponds to the amino acid site, and any combination thereof, by using the OsPPO1 amino acid sequence as reference standard; or
the first target gene is TIR1, and the specific site of gene is selected from sites F93, F357, C413, and S448 in an Oryza sativa OsTIR1 protein amino acid sequence, and amino acid sites in a TIR1 protein of another plant, which correspond to the amino acid sites by using the OsTIR1 amino acid sequence as reference standard; or the crRNA or sgRNA targets a target sequence comprising a sequence encoding an OsTIR1 amino acid sequence site selected from F93, F357, C413, S448, and any combination thereof, and a target sequence comprising a sequence encoding an amino acid site in a TIR1 protein of another plant which corresponds to the amino acid site, and any combination thereof, by using the OsTIR1 amino acid sequence as reference standard.

145. A non-transgenic transient editing system, which uses the method of claim 136.

146. A genetically modified plant obtained by the method according to claim 142, wherein the genome thereof comprises:
1) an editing event of a first target gene;
2) an editing event of the first target gene and an editing event of at least one second target gene; or
3) at least one second target gene editing event, wherein the editing event of the first target gene has been removed by genetic separation;
wherein, the genetically modified plant is obtained in a non-transgenic manner.

147. A new plant gene mutation obtained by the method according to claim 142.

148. A new mutation generated in a plant, which comprises one or a combination of two or more of the following types:
substitution of aspartic acid at a site corresponding to Arabidopsis ALS376 with any other amino acid, substitution of tryptophan at a site corresponding to Arabidopsis ALS574 with any other amino acid, substitution of serine at a site corresponding to Arabidopsis ALS653 with any other amino acid, or substitution of serine at a site corresponding to Arabidopsis ALS654 with any other amino acid; or substitution of tryptophan at a site corresponding to Alopecurus myosuroides ACCase2027 with any other amino acid.

149. A protein or biologically active fragment thereof, comprising the new mutation of claim 147.

150. A nucleic acid, comprising a nucleic acid sequence or complementary sequence thereof that encodes the protein or biologically active fragment thereof of claim 149.

151. A method for producing a plant with improved resistance or tolerance to herbicides, which comprises transforming or transfecting a plant cell with a recombinant expression vector or an expression cassette comprising the nucleic acid of claim 150 and regenerating the transformed or transfected plant cell into a plant.

152. A method for controlling weeds in a plant cultivation site, wherein the plant is the plant of claim 146, and the method comprises applying to the cultivation site one or more herbicides in an effective amount to control the weeds.

153. A method for controlling weeds in a plant cultivation site, wherein the plant is the plant produced by the method of claim 151, and the method comprises applying to the cultivation site one or more herbicides in an effective amount to control the weeds.


SEQUENCE LISTING (157 sequences)

SEQ ID NO: 1 - protein, 670 aa, Arabidopsis thaliana
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGASMEIHQALTRSSSIRNVLPRHEQGGVFAAEGYARSSGKPGICIATSGPGATNLVSGLADALLDSVPLVAITGQVPRRMIGTDAFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVENLPVKVLLLNNQHLGMVMQWEDRFYKANRAHTFLGDPAQEDEIFPNMLLFAAACGIPAARVTKKADLREAIQTMLDTPGPYLLDVICPHQEHVLPMIPSGGTFNDVITEGDGRIKY

SEQ ID NO: 2 - DNA, 2013 bp, Arabidopsis thaliana
atggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatactga

SEQ ID NO: 3 - protein, 2320 aa, Alopecurus myosuroides
MGSTHLPIVGFNASTTPSLSTLRQINSAAAAFQSSSPSRSSKKKSRRVKSIRDDGDGSVPDPAGHGQSIRQGLAGIIDLPKEGASAPDVDISHGSEDHKASYQMNGILNESHNGRHASLSKVYEFCTELGGKTPIHSVLVANNGMAAAKFMRSVRTWANDTFGSEKAIQLIAMATPEDMRINAEHIRIADQFVEVPGGTNNNNYANVQLIVEIAERTGVSAVWPGWGHASENPELPDALTAKGIVFLGPPASSMNALGDKVGSALIAQAAGVPTLAWSGSHVEIPLELCLDSIPEEMYRKACVTTADEAVASCQMIGYPAMIKASWGGGGKGIRKVNNDDEVKALFKQVQGEVPGSPIFIMRLASQSRHLEVQLLCDEYGNVAALHSRDCSVQRRHQKIIEEGPVTVAPRETVKELEQAARRLAKAVGYVGAATVEYLYSMETGEYYFLELNPRLQVEHPVTESIAEVNLPAAQVAVGMGIPLWQIPEIRRFYGMDNGGGYDIWRKTAALATPFNFDEVDSQWPKGHCVAVRITSENPDDGFKPTGGKVKEISFKSKPNVWGYFSVKSGGGIHEFADSQFGHVFAYGETRSAAITSMSLALKEIQIRGEIHTNVDYTVDLLNAPDFRENTIHTGWLDTRIAMRVQAERPPWYISVVGGALYKTITTNAETVSEYVSYLIKGQIPPKHISLVHSTISLNIEESKYTIEIVRSGQGSYRLRLNGSLIEANVQTLCDGGLLMQLDGNSHVIYAEEEAGGTRLLIDGKTCLLQNDHDPSRLLAETPCKLLRFLIADGAHVDADVPYAEVEVMKMCMPLLSPAAGVINVLLSEGQAMQAGDLIARLDLDDPSAVKRAEPFEGSFPEMSLPIAASGQVHKRCAASLNAARMVLAGYDHAANKVVQDLVWCLDTPALPFLQWEELMSVLATRLPRRLKSELEGKYNEYKLNVDHVKIKDFPTEMLRETIEENLACVSEKEMVTIERLVDPLMSLLKSYEGGRESHAHFIVKSLFEEYLSVEELFSDGIQSDVIERLRLQYSKDLQKVVDIVLSHQGVRNKTKLILALMEKLVYPNPAAYRDQLIRFSSLNHKRYYKLALKASELLEQTKLSELRTSIARNLSALDMFTEEKADFSLQDRKLAINESMGDLVTAPLPVEDALVSLFDCTDQTLQQRVIQTYISRLYQPQLVKDSIQLKYQDSGVIALWEFTEGNHEKRLGAMVILKSLESVSTAIGAALKDASHYASSAGNTVHIALLDADTQLNTTEDSGDNDQAQDKMDKLSFVLKQDVVMADLRAADVKVVSCIVQRDGAIMPMRRTFLLSEEKLCYEEEPILRHVEPPLSALLELDKLKVKGYNEMKYTPSRDRQWHIYTLRNTENPKMLHRVFFRTLVRQPSAGNRFTSDHITDVEVGHAEEPLSFTSSSILKSLKIAKEELELHAIRTGHSHMYLCILKEQKLLDLVPVSGNTVVDVGQDEATACSLLKEMALKIHELVGARMHHLSVCQWEVKLKLVSDGPASGSWRVVTTNVTGHTCTVDIYREVEDTESQKLVYHSTALSSGPLHGVALNTSYQPLSVIDLKRCSARNNKTTYCYDFPLTFEAAVQKSWSNISSENNQCYVKATELVFAEKNGSWGTPIIPMQRAAGLNDIGMVAWILDMSTPEFPSGRQIIVIANDITFRAGSFGPREDAFFEAVTNLACEKKLPLIYLAANSGARIGIADEVKSCFRVGWTDDSSPERGFRYIYMTDEDHDRIGSSVIAHKMQLDSGEIRWVIDSVVGKEDGLGVENIHGSAAIASAYSRAYEETFTLTFVTGRTVGIGAYLARLGIRCIQRIDQPIILTGFSALNKLLGREVYSSHMQLGGPKIMATNGVVHLTVPDDLEGVSNILRWLSYVPANIGGPLPITKSLDPIDRPVAYIPENTCDPRAAISGIDDSQGKWLGGMFDKDSFVETFEGWAKTVVTGRAKLGGIPVGVIAVETQTMMQLVPADPGQPDSHERSVPRAGQVWFPDSATKTAQAMLDFNREGLPLFILANWRGFSGGQRDLFEGILQAGSTIVENLRTYNQPAFVYIPKAAELRGGAWVVIDSKINPDRIECYAERTAKGNVLEPQGLIEIKFRSEELKECMGRLDPELIDLKARLQGANGSLSDGESLQKSIEARKKQLLPLYTQIAVRFAELHDTSLRMAAKGVIRKVVDWEDSRSFFYKRLRRRLSEDVLAKEIRGVIGEKFPHKSAIELIKKWYLASEAAAAGSTDWDDDDAFVAWRENPENYKEYIKELRAQRVSRLLSDVAGSSSDLQALPQGLSMLLDKMDPSKRAQFIEEVMKVLK

SEQ ID NO: 4 - DNA, 6963 bp, Alopecurus myosuroides
atgggatccacacatctgcccattgtcgggtttaatgcatccacaacaccatcgctatccactcttcgccagataaactcagctgctgctgcattccaatcttcgtccccttcaaggtcatccaagaagaaaagccgacgtgttaagtcaataagggatgatggcgatggaagcgtgccagaccctgcaggccatggccagtctattcgccaaggtctcgctggcatcatcgacctcccaaaggagggcgcatcagctccagatgtggacatttcacatgggtctgaagaccacaaggcctcctaccaaatgaatgggatactgaatgaatcacataacgggaggcacgcctctctgtctaaagtttatgaattttgcacggaattgggtggaaaaacaccaattcacagtgtattagtcgccaacaatggaatggcagcagctaagttcatgcggagtgtccggacatgggctaatgatacatttgggtcagagaaggcgattcagttgatagctatggcaactccggaagacatgagaataaatgcagagcacattagaattgctgatcagtttgttgaagtacctggtggaacaaacaataacaactatgcaaatgtccaactcatagtggagatagcagagagaactggtgtctccgccgtttggcctggttggggccatgcatctgagaatcctgaacttccagatgcactaactgcaaaaggaattgtttttcttgggccaccagcatcatcaatgaacgcactaggcgacaaggttggttcagctctcattgctcaagcagcaggggttcccactcttgcttggagtggatcacatgtggaaattccattagaactttgtttggactcgatacctgaggagatgtataggaaagcctgtgttacaaccgctgatgaagcagttgcaagttgtcagatgattggttaccctgccatgatcaaggcatcctggggtggtggtggtaaagggattagaaaggttaataatgatgacgaggtgaaagcactgtttaagcaagtacagggtgaagttcctggctccccgatatttatcatgagacttgcatctcagagtcgtcatcttgaagtccagctgctttgtgatgaatatggcaatgtagcagcacttcacagtcgtgattgcagtgtgcaacgacgacaccaaaagattatcgaggaaggaccagttactgttgctcctcgtgaaacagtgaaagagctagagcaagcagcaaggaggcttgctaaggccgtgggttacgtcggtgctgctactgttgaatatctctacagcatggagactggtgaatactattttctggagcttaatccacggttgcaggttgagcacccagtcaccgagtcgatagctgaagtaaatttgcctgcagcccaagttgcagttgggatgggtatacccctttggcagattccagagatcagacgtttctacggaatggacaatggaggaggctatgatatttggaggaaaacagcagctctcgctactccattcaactttgatgaagtagattctcaatggccgaagggtcattgtgtggcagttaggataaccagtgagaatccagatgatggattcaagcctactggtggaaaagtaaaggagataagttttaaaagtaagccaaatgtctggggatatttctcagttaagtctggtggaggcattcatgaatttgcggattctcagtttggacacgtttttgcctatggagagactagatcagcagcaataaccagcatgtctcttgcactaaaagagattcaaattcgtggagaaattcatacaaacgttgattacacggttgatctcttgaatgccccagacttcagagaaaacacgatccataccggttggctggataccagaatagctatgcgtgttcaagctgagaggcctccctggtatatttcagtggttggaggagctctatataaaacaataaccaccaatgcggagaccgtttctgaatatgttagctatctcatcaagggtcagattccaccaaagcacatatcccttgtccattcaactatttctttgaatatagaggaaagcaaatatacaattgagattgtgaggagtggacagggtagctacagattgagactgaatggatcacttattgaagccaatgtacaaacattatgtgatggaggccttttaatgcagctggatggaaatagccatgttatttatgctgaagaagaagcgggtggtacacggcttcttattgatggaaaaacatgcttgctacagaatgaccatgatccgtcaaggttattagctgagacaccctgcaaacttcttcgtttcttgattgccgatggtgctcatgttgatgctgatgtaccatacgcggaagttgaggttatgaagatgtgcatgcccctcttgtcgcctgctgctggtgtcattaatgttttgttgtctgagggccaggcgatgcaggctggtgatcttatagcgagacttgatctcgatgacccttctgctgtgaagagagccgagccatttgaaggatcttttccagaaatgagccttcctattgctgcttctggccaagttcacaaaagatgtgctgcaagtttgaacgctgctcgaatggtccttgcaggatatgaccatgcggccaacaaagttgtgcaagatttggtatggtgccttgatacacctgctcttcctttcctacaatgggaagagcttatgtctgttttagcaactagacttccaagacgtcttaagagcgagttggagggcaaatacaatgaatacaagttaaatgttgaccatgtgaagatcaaggatttccctaccgagatgcttagagagacaatcgaggaaaatcttgcatgtgtttccgagaaggaaatggtgacaattgagaggcttgttgaccctctgatgagcctgctgaagtcatacgagggtgggagagaaagccatgcccactttattgtcaagtccctttttgaggagtatctctcggttgaggaactattcagtgatggcattcagtctgacgtgattgaacgcctgcgcctacaatatagtaaagacctccagaaggttgtagacattgttttgtctcaccagggtgtgagaaacaaaacaaagctgatactcgcgctcatggagaaactggtctatccaaaccctgctgcctacagagatcagttgattcgcttttcttccctcaaccataaaagatattataagttggctcttaaagctagtgaacttcttgaacaaaccaagctcagcgaactccgcacaagcattgcaaggaacctttcagcgctggatatgttcaccgaggaaaaggcagatttctccttgcaagacagaaaattggccattaatgagagcatgggagatttagtcactgccccactgccagttgaagatgcacttgtttctttgtttgattgtactgatcaaactcttcagcagagagtgattcagacatacatatctcgattataccagcctcaacttgtgaaggatagcatccagctgaaatatcaggattctggtgttattgctttatgggaattcactgaaggaaatcatgagaagagattgggtgctatggttatcctgaagtcactagaatctgtgtcaacagccattggagctgctctaaaggatgcatcacattatgcaagctctgcgggcaacacggtgcatattgctttgttggatgctgatacccaactgaatacaactgaagatagtggtgataatgaccaagctcaagacaagatggataaactttcttttgtactgaaacaagatgttgtcatggctgatctacgtgctgctgatgtcaaggttgttagttgcattgttcaaagagatggagcaatcatgcctatgcgccgtaccttcctcttgtcagaggaaaaactttgttacgaggaagagccgattcttcggcatgtggagcctccactttctgcacttcttgagttggataaattgaaagtgaaaggatacaatgagatgaagtatacaccgtcacgtgatcgtcagtggcatatatacacacttagaaatactgaaaatccaaaaatgctgcacagggtatttttccgaacacttgtcagacaacccagtgcaggcaacaggtttacatcagaccatatcactgatgttgaagtaggacacgcagaggaacctctttcatttacttcaagcagcatattaaaatcgttgaagattgctaaagaagaattggagcttcacgcgatcaggactggccattctcatatgtacttgtgcatattgaaagagcaaaagcttcttgaccttgttcctgtttcagggaacactgttgtggatgttggtcaagatgaagctactgcatgctctcttttgaaagaaatggctttaaagatacatgaacttgttggtgcaagaatgcatcatctttctgtatgccagtgggaagtgaaacttaagttggtgagcgatgggcctgccagtggtagctggagagttgtaacaaccaatgttactggtcacacctgcactgtggatatctaccgggaggtcgaagatacagaatcacagaaactagtataccactccaccgcattgtcatctggtcctttgcatggtgttgcactgaatacttcgtatcagcctttgagtgttattgatttaaaacgttgctctgccaggaacaacaaaactacatactgctatgattttccattgacatttgaagctgcagtgcagaagtcgtggtctaacatttccagtgaaaacaaccaatgttatgttaaagcgacagagcttgtgtttgctgaaaagaatgggtcgtggggcactcctataattcctatgcagcgtgctgctgggctgaatgacattggtatggtagcctggatcttggacatgtccactcctgaatttcccagcggcagacagatcattgttatcgcaaatgatattacatttagagctggatcatttggcccaagggaagatgcatttttcgaagctgtaaccaacctggcttgtgagaagaagcttccacttatctacttggctgcaaactctggtgctcggattggcattgctgatgaagtaaaatcttgcttccgtgttggatggactgatgatagcagccctgaacgtggatttaggtacatttatatgactgacgaagaccatgatcgtattggctcttcagttatagcacacaagatgcagctagatagtggcgagatcaggtgggttattgattctgttgtgggaaaagaggatggactaggtgtggagaacatacatggaagtgctgctattgccagtgcctattctagggcgtacgaggagacatttacacttacattcgttactggacgaactgttggaatcggagcctatcttgctcgacttggcatacggtgcatacagcgtattgaccagcccattattttgaccgggttttctgccctgaacaagcttcttgggcgggaggtgtacagctcccacatgcagttgggtggtcccaaaatcatggcgacgaatggtgttgtccatctgactgttccagatgaccttgaaggtgtttctaatatattgaggtggctcagctatgttcctgcaaacattggtggacctcttcctattacaaaatctttggacccaatagacagacccgttgcatacatccctgagaatacatgtgatcctcgtgcagccatcagtggcattgatgacagccaagggaaatggttgggtggcatgtttgacaaagacagttttgtggagacatttgaaggatgggcgaagacagtagttactggcagagcaaaacttggagggattcctgttggtgttatagctgtggagacacagaccatgatgcagctcgtccccgctgatccaggccagcctgattcccacgagcggtctgttcctcgtgctgggcaagtttggtttccagattctgctaccaagacagcgcaggcgatgttggacttcaaccgtgaaggattacctctgttcatacttgctaactggagaggcttctctggagggcaaagagatctttttgaaggaattctgcaggctgggtcaacaattgttgagaaccttaggacatacaatcagcctgcctttgtatatatccccaaggctgcagagctacgtggaggagcctgggtcgtgattgatagcaagataaacccagatcgcatcgagtgctatgctgagaggactgcaaagggtaatgttctcgaacctcaagggttgattgagatcaagttcaggtcagaggaactcaaagaatgcatgggtaggcttgatccagaattgatagatctgaaagcaagactccagggagcaaatggaagcctatctgatggagaatcccttcagaagagcatagaagctcggaagaaacagttgctgcctctgtacacccaaatcgcggtacgttttgcggaattgcacgacacttcccttagaatggctgctaaaggtgtgatcaggaaagttgtagactgggaagactctcggtctttcttctacaagagattacggaggaggctatccgaggacgttctggcaaaggagattagaggtgtaattggtgagaagtttcctcacaaatcagcgatcgagctgatcaagaaatggtacttggcttctgaggcagctgcagcaggaagcaccgactgggatgacgacgatgcttttgtcgcctggagggagaaccctgaaaactataaggagtatatcaaagagcttagggctcaaagggtatctcggttgctctcagatgttgcaggctccagttcggatttacaagccttgccgcagggtctttccatgctactagataagatggatccctctaagagagcacagtttatcgaggaggtcatgaaggtcctgaaatga

SEQ ID NO: 5 - protein, 446 aa, Oryza sativa
MPPTPTPTATTGAVSAAAAAGENAGFRLVGHRRFVRANPRSDRFQALAFHHVELWCADAASAAGRFAFALGAPLAARSDLSTGNSAHASLLLRSASVAFLFTAPYGGDHGVGADAATTASIPSFSPGAARRFAADHGLAVHAVALRVADAADAFRASVAAGARPAFQPADLGGGFGLAEVELYGDVVLRFVSHPDGADAPFLPGFEGVSNPGAVDYGLRRFDHVVGNVPELAPVAAYISGFTGFHEFAEFTAEDVGTAESGLNSVVLANNAETVLLPLNEPVHGTKRRSQIQTYLDHHGGPGVQHIALASDDVLGTLREMRARSAMGGFEFLAPPPPNYYDGVRRRAGDVLSEEQINECQELGVLVDRDDQGVLLQIFTKPVGDRPTFFLEMIQRIGCMEKDESGQEYQKGGCGGFGKGNFSELFKSIEEYEKSLEAKQAPTVQGS

SEQ ID NO: 6 - DNA, 2099 bp, Oryza sativa
atgcctcccactcccacccccaccgccaccaccggcgccgtctcggccgctgcggcggcgggggagaacgcggggttccgcctcgtcgggcaccgccgcttcgtccgcgccaacccgcggagcgaccggttccaggcgctcgcgttccaccacgtcgagctctggtgcgccgacgccgcgtccgccgcgggccggttcgccttcgccctgggcgcgccgctcgccgccaggtccgacctctccacggggaactccgcgcacgcctccctcctcctccgctccgcctccgtcgcgttcctcttcaccgccccctacggcggcgaccacggcgtcggcgcggacgcggccaccaccgcctccatcccttccttctccccaggcgccgcgcggaggttcgccgcggaccacggcctcgcggtgcacgccgtggcgctgcgcgtcgccgacgcggccgacgccttccgcgccagcgtcgcggccggtgcgcgcccggcgttccagcccgccgacctcggcggtggcttcggcctcgcggaggtggagctctacggcgacgtcgtgctccgcttcgtcagccacccggacggcgccgacgcgcccttcctcccgggtttcgagggcgtcagcaacccgggcgccgtggactacggcctccgccggttcgaccacgtcgtcggcaacgtgccggagctcgctccggtagccgcgtacatctccgggttcaccgggttccacgagttcgccgagttcaccgccgaggacgtgggcaccgccgagagcggcctcaactcggtggtgctcgccaacaacgcggagaccgtgctgctgccgctcaacgagccggtgcacggcaccaagcggcggagccagatacagacgtacctggaccaccacggcggcccgggggtgcagcacatcgcgctggccagcgacgacgtgctcgggacgctgagggagatgcgggcgcgctccgccatgggcggcttcgagttcttggcgccgccgccgcccaactactacgacggcgtgcggcggcgcgccggggacgtgctctcggaggagcagatcaacgagtgccaggagctcggggtgctcgtggacagggatgaccagggggtgttgctccagatcttcaccaagccagtaggagacaggtaaaatcctcacctctttcatgatgaaaatggcttatgaattcagatttgcagttatttgttggcacatagcatcgattaggcgcagaaaggtgtcaagcattatgaaattaatccagaatgcttgaataatacagtataatatatgatagtgagctctgtgatactccatggatactctttatgtgtctccatgaatccatgatgcgcctttctgaagattgtgacactagaaagggaataaagctgaatgtgcataggaaaaaaatgaaaagccaatgtgtgtctgtttatgccttcttgcaagcatatcccagttcctttttgccggcatgttgtaatgcagatagccagccacatatagctacttaattagtgagtactccctctcacaatgtaagtcattctagtattttccacattcatattgatgctaatctatctagattcattagcatcaatatgaatatgggaaatactagaatgacttacattgtgaaacggaggaagtattacttactacatctaaggtccatggattcctttttttacaaaagaaagaaagaatcttatggcaactccatcagcataaaccagcaatgctgctgggaacaacttaaactttaggttcaggaggttgtaattgtctttaagcttaatagtctgattcagtcagtattctaatttctgctgcatctttgctattgttatttcctctctgtgactccaaatctaactggatcagctatttcactcaggccaacctttttcttggagatgatacaaaggattgggtgcatggagaaggatgagagtgggcaggagtaccagaagggcggctgcggcgggtttgggaagggcaacttctcggagctgttcaagtccattgaggagtatgagaaatcccttgaagccaagcaagcccctacagttcaaggatcctag

SEQ ID NO: 7 - protein, 536 aa, Oryza sativa
MAAAAAAMATATSATAAPPLRIRDAARRTRRRGHVRCAVASGAAEAPAAPGARVSADCVVVGGGISGLCTAQALATKHGVGDVLVTEARARPGGNITTAERAGEGYLWEEGPNSFQPSDPVLTMAVDSGLKDDLVFGDPNAPRFVLWEGKLRPVPSKPGDLPFFDLMSIPGKLRAGLGALGVRAPPPGREESVEDFVRRNLGAEVFERLIEPFCSGVYAGDPSKLSMKAAFGKVWRLEDTGGSIIGGTIKTIQERGKNPKPPRDPRLPTPKGQTVASFRKGLTMLPDAITSRLGSKVKLSWKLTSITKSDNKGYALVYETPEGVVSVQAKTVVMTIPSYVASDILRPLSSDAADALSIFYYPPVAAVTVSYPKEAIRKECLIDGELQGFGQLHPRSQGVETLGTIYSSSLFPNRAPAGRVLLLNYIGGSTNTGIVSKTESELVEAVDRDLRKMLINPKAVDPLVLGVRVWPQAIPQFLIGHLDHLEAAKSALGKGGYDGLFLGGNYVAGVALGRCVEGAYESASQISDYLTKYAYK

SEQ ID NO: 8 - DNA, 3246 bp, Oryza sativa
atggccgccgccgccgcagccatggccaccgccacctccgccacggcagcgccgccgctccgcattcgcgacgccgcgaggaggacccgccgacgcggccacgttcgctgcgccgtcgccagcggcgcggccgaggcgcccgcggcgcccggggcgcgggtgtcggcggactgcgtcgtggtgggcggcggcatcagcgggctctgcaccgcgcaggcgctggccacaaagcacggcgtcggcgacgtgctcgtcacggaggcccgcgcccgccccggcggcaacatcaccaccgccgagcgcgccggcgagggctacctctgggaggaggggcccaacagcttccagccttccgaccccgtcctcaccatggccgtacgtcttcttgctccccttctcttctgattctctcgcggcggagacgacatgaatgggaatggtggtgcatggattggggcgcgcaggtggacagcgggctcaaggacgatctcgtgttcggggaccccaacgcgccgcggttcgtgctgtgggaggggaagctaaggccggtgccgtccaagcccggcgacctgccgttcttcgacctcatgagcatccccggcaagctcagggccggccttggcgcgctcggcgttcgagcgccacctccagtttgtgtgctctccccgctgtgcattcttgattcacttgtgaaattcgattgtgctgagcgtttccggcgaaggtttcaggggcgtgaggagtcggtggaggacttcgtgcggcgcaacctcggcgcggaggtctttgagcgcctcattgagcctttctgctcaggtgtttattgtagtgtgcaattgctgttttgtttttgatgattcagataagaatacggtgatttcggtgcttaggtgtgtatgctggtgatccttcaaagctcagtatgaaggctgcatttgggaaggtgtggaggctggaggatactggaggtagcattattggtggaaccatcaaaacaatccaggagagggggaaaaaccccaaaccgccgagggatccgtgagtgagaaattgccttctttgttggattaattgtccattgtgttacactgatatgccttcaccatttttagccgccttccaacgccaaaggggcagacagttgcatctttcaggaagggtctgactatgctcccggatgctattacatctaggtttgttatcattgtctttgtaatttacctagttcttcaactatggatattaggtgctgtagattgttcagatagatgcacattgtacaacaatctaggtagattgattgctatggcttgttgaattaactgtttcacttgcatcattgcctcagcacatatgaagcatatggatagattcttcaatcatttatccctcaataacacaattttgacaccagtgcttctcctttttttcatcctgcttcatctggctatccacaatataattaagcatacaaaagaggcactctttgatggacaattcatagtgttgtgggttaatattcatttgcattctttgagggacaattcgttacaccctaacatgaactagtaatgattggggtgcttaaccatttgttctgcatttcctccattttcaggttgggtagcaaagtcaaactttcatggaagttgacaagcattacaaagtcagacaacaaaggatatgcattagtgtatgaaacaccagaaggggtggtctcggtgcaagctaaaactgttgtcatgaccatcccatcatatgttgctagtgatatcttgcggccactttcagtaagttatatatatttaattaactttctgttcccaaaatacactgcagcacttcattgcttcctgaggtcctcgattcatttttcggtagacaggaagtagtattcatttgcactttttaagggattaattcaacatatccactggaaatatacatatcctacacatcctgtcaacatacttgctaaacagcattttgtttgagttgactggcatctcagcagccaattactatctttaggggacaagccacattcttaataaatcctgtcggaaatcactttttgatttttatagattagtgttgtcatagaatttggcttggtgatctacttggtaaggttaactgattcacaagtcggacaatttcttcaccaatctagcagtatgtttaagtgtgttggtacttaattctaaatgtcctgcgcatggtaacatatcatatgcaaaaattcctcagtaaccgaaatttatactgtaagttttaactgtctttacactgttaattttagacatacttcttccttgcttgttcattgaacttgtttcccccttccacagagtgatgcagcagatgctctgtcaatattctattatccaccagttgctgctgtaactgtttcatatccaaaagaagcaattagaaaagaatgcttaattgacggagagctccagggtttcggccagctgcatccgcgtagtcagggagttgagactttaggtacttataggaattcaaccttattattcttctaacatataaatgaactaatctttcttgtctagtttgcatttattgtggattaagtttggttatattgttcttacaagtttgtggtattattttgtataggaacaatatatagctcatcactctttccaaatcgtgctccagctggaagggtgttacttctgaactacataggaggttctacaaatacagggattgtttccaaggtatcgctgtcaagttgtttattttgcgactatatgattacagtatcctgtttttcaactccagctgctgttagactgtcataataaatctgctactacatgtttgcacactatttgactgcatttaaaaactcagatagcctatatttttagttgcctgctactgggtgtatttctaatgatcccatcatgtttgcagactgaaagtgagctggtagaagcagttgaccgtgacctcaggaagatgctgataaatcctaaagcagtggaccctttggtccttggcgtccgggtatggccacaagccataccacagttcctcattggccatcttgatcatcttgaggctgcaaaatctgccctgggcaaaggtggttatgatggattgttcctcggagggaactatgttgcaggagttgccctgggccgatgcgttgaaggtgcatatgagagtgcctcacaaatatctgactacttgaccaagtacgcctacaagtga

SEQ ID NO: 9 - protein, 587 aa, Oryza sativa
MGRGGSRAACAAAAPPWHSLPDEVWEHAFSFLPAAADRGAAAGACSSWLRAERRSRRRLAVANCYAAAPRDAVERFPSVRAAEVKGKPHFADFGLVPPAWGAAAAPWIAAAADGWPLLEELSFKRMVVTDECLEMIAASFRNFQVLRLVSCDGFSTAGLAAIAAGCRHLRELDLQENEIEDCSIHWLSLFPESFTSLVTLNFSCLEGEVNITVLERLVTRCHNLKTLKLNNAIPLDKLASLLHKAPQLVELGTGKFSADYHSDLFAKLEAAFGGCKSLRRLSGAWDAVPDYLPAFYCVCEGLTSLNLSYATVRGPELIKFISRCRNLQQLWVMDLIEDHGLAVVASSCNKLQELRVFPSDPFGAGFLTERGLVDVSASCPMLESVLYFCRRMTNEALITIAKNRPNFTCFRLCILEPHTPDYITREPLDAGFSAIVESCRGLRRLSISGLLTDLVFKSIGAHADRLEMLSIAFAGNSDLGLHYILSGCKSLKKLEIRDCPFGDKPLLANAAKLETMRSLWMSSCLLTLGACRQLARKMPRLSVEIMNDPGRSCPLDSLPDETPVEKLYVYRTIAGPRSDTPACVQIV

SEQ ID NO: 10 - DNA, 5791 bp, Oryza sativa
atggggcgcggcggctcgcgcgcggcgtgcgccgccgcggcgccgccgtggcactcgctcccggacgaggtctgggagcacgccttctccttcctccccgccgccgcggacaggggcgccgcggcgggggcgtgcagctcgtggctccgcgccgagcgccggtcgcgccgccgcctcgccgtcgccaactgctacgccgccgcgccgcgggacgccgtcgagcggttcccgtccgtgcgcgccgccgaggtcaagggcaagccccacttcgccgacttcggcctcgtcccccccgcctggggcgccgccgcggcgccgtggatcgccgccgccgccgacgggtggccgctgctcgaggagctcagcttcaagcgcatggtcgtcaccgacgagtgcctcgagatgatcgccgcgtccttcaggaacttccaggtgctccgcctcgtctcctgcgacggcttcagcaccgcgggcctcgccgccattgctgccggttgcaggtgaggtggagagtgttgtttgggtcaaatctttttagctaaataagcatgggatctttgggttgttcttggatttgagaggaatctttcacggcccaagctttgattcgtcttctttttagctaaaacatgacagatttcttggtgccatttctttcttcacagtgttctttagtgagttcatggtctagctgaagtgatttgttcttccatgattcctatattaactttgttttgaacttcattgagtagtatgtggaagtagatgtggctttctgctaagatttgatctattcttggtgtttaatttgcaaagggacagtttttttttctgtttacttgtgtatgatctggatttacttttggtgtttcctaagttgaagaccaagaagtcttcttgtgacagtattttagatgcttataacgtgttaatgtcaaaggaatgattcccatgaaaattctgctgtgatttgtacattttattaggagttaagtaacagagtgaattgagtgatgctcacgtcttctttaaatcttagctatgatttgttcctaggaagctttgacttgtatttgagtgcctggtttgcgaagatgtgattctcttgcacggatctgtgtgattgacagaagaaatatttctacctgtttaagcaatgatggatccatctctttgtttctttttgtgctttttacccaaaagaatgggatatttcaagtttacaaacaactggtgatgcgcctttataattttgggcttttttaaaagaatgtattcttcccatcatgagttactagatctgagttgaactgttttcctttatattttcctgatatttaagtgaatttgttgagttcctatcctgatttgaacttccagattctaaagaaccaggtcttttaagcatgtttttgtcatgtagattcccagtttatcccctccccctccttttgtgctcaagaatctactaccttttatcttgcttttggtgagggaaagtgatttttgctccaatatttgtgatgtcctcatgtaatggacagttaaaacaaacagatttgttcttgcgagtatcttctggactccccccagaaaagaattaggaatgtgtgggattcctatcattggggaatttatatcctaaaccagatagaaggatctaatagtgggagtttcgatcctgattcatccattagtagtagtttaatatgccaaagcagtatcatttgcatcattttgaggaactcgaaacagttgaactgtgaagtttaactactgtacacttcaaacaggcagattagaataggttgtgagataactaaatttggttcggtgatcgtataaggccatattcttggatattaggacgaaatgagcctgagcaccacatgtgtacttgctgatggttgagaacttagcatagatactagtttttatgctagactagagggctactggtacattcacttgtttgataaatccagatgtatttaagttttgattcaaaatattttgactctagattggagactacattttagagcattcaaaatttacaatggaagcaggattttagcaaaactattttcatacatttactctgttattgagccagaatcattttttagttcttgctacttttttgttaacactgttatgcatgtaaactcagcgttacatttatttcaattacaaggatctacacttgtagctctgggtgcagatggtagttaacattgatgaatcttagctatatcgaaatactatcttctaagtacaaacttttcttgtgtgattcttatccttgttgttactgttgctacttatatccattgattgagcatgctttgaattaccttaagaatttctctctccagtcaaatttatttgactttgagagaactgtgtttatttctcccaacgtcaaatgttttgaattttgaatggacagagtatgtagtagtgggcagcacattggtgccctagctgcaaataaaaccttttttttttgttaaaacaatgattatgaggatgtcggtttacactgtccactcagaaatttgacttgattatggatagggtacaccttgttgttttagatggttccttcttaacaagatgctagattttgatgatttgattctttacaaggagtagtctaaaatactggaagtgtcaacttaaaaagttcaaacttgccctagccatacgtgatacattagacaactgatgtgctgtgttatctgcaactgacaattattgttgttgctatctcctttttttgctttagagtaggcaggtattttgtgtcaactaaaagtgtgtttcacgtgttgcctttcttaataccttttgaataatagggtataagaaaattttcatgaatggtttgctttccaattgttgatgttgctttccttcaccttattttttaaaccaccatgattaatctatttatctaggtaatattgctcatgctgattcatgatacgtggtaaaaaaataggtcttttcaactttcagaggctgctggactccatttatttagttgattttgatccctacaccgattttatccattggtttatattcactgctaacatatgatactcgctatcatttacttcactgttgatcatcaatgttttaattttgaccattattaacttttatatgattaatttagtattaatgaaagttgtgcggttgtatttatggcatgacacacatttctaacatggcatacttttaagcatacataactttttttgtaaaaaggacaagcgatcaaaggtaaaaaggtgaatagcatacagtgagaagtatttgagaccaaagggagtataatcattatctactaaacaaatacaataccattcattataagctgagttcgcatagtgtaattgaaagtggggaatgctagtatagctcacacatgaaatcaagtccctgtgaaatgatagtaaaccctacgtgttcattgttttagtcttatgatatgttccagttatgtttgccatggttgttgaatgcagtagtcaaagtttgcttctgtttgtttgagtggattctcatttaccttgccttttccaccatatactcatatgggctttgtttcacaagtgataacattggttaatggctatatgctatccttacagtttcattcttcttttaagatatgagtcaccatgcacatttcttcaacttctgattttgtctaatattgcagacacctaagagaacttgacctgcaagagaacgagattgaggattgttctattcattggctcagcctcttcccggaatcgttcacttctctagtaactctaaacttttcatgcttagagggggaggtcaatatcactgtacttgaacggttagtgaccagatgtcacaacctgaagactcttaagctcaacaatgctatcccccttgacaagcttgctagcctccttcataaggctcctcagctagttgaactcggaactggcaaattctctgctgattaccattccgatctgtttgcaaagctggaggcggcgtttggaggttgtaaaagcttgagaaggctttctggggcttgggatgctgttccagattatctgccagcattctattgtgtatgtgaaggcctcacatcacttaatctgagttatgctactgtgcgaggtcctgagctcatcaaattcattagtagatgcagaaatttgcaacaattatgggtatgcaactcttgtcaattcttttttactactatgtattcatcccattcgaatctgttaacttttgaaaaactgttgcatatctctttgcaatacaagcacttgattccctacaagtggattttgcaaattttttcttttcttctgtgacctccagtaaaaaagttcctgatgttttcttacttatcatggtcactagtaaaagtccgactacatgtattcttttttcacaatgtagttttatcaattatattggtatagggctgcaatagcattttatgatgaaactgactgcacttttcccatgacaacaagaatttgtggcttggaataatctattgagtgttaaatcatgcatatgtttttcatgtttgtcaatacagttttgtaggatgcatgttacattcaatggcatgctgttgggtgcctccaatgaatagattgaaattaccactcttatcttttggggaagtaggatatttatatggaaaaaacatagatgacaatggtatcctatccttattagaatcattgtgaaaagactgccagatgaatccatttgctctagggcagcattctttgcctttcttggcaaactgagttattgtctgaaccctcatagtttccatcctttaactgttaactgctagatgagttgcttgctctacggcagatattttaaactgaattatgatctgaaccccaatggctattaacctggtcatgttagctctttatgcaatcttttttctctatatttttcttctaaaagttaagctgcaatttttcaggtgatggacctcattgaggatcatggtttagctgttgtggcatcatcttgcaataaacttcaagagttgcgggtcttcccttctgacccttttggtgcaggattcttgactgaaagaggtcttgttgatgtctctgcaagttgtccaatgttggagtcagtgctctacttctgcagacggatgacaaatgaggcacttattaccattgcaaagaaccgtcccaacttcacttgcttccgcctatgcatccttgagccacacactccagactacatcacacgggagcctcttgatgcaggtttcagcgccattgtggagtcatgcaggggccttaggcgtctctctatctcaggccttctcacagatcttgtgtttaaatccattggggcacatgctgatcgtcttgagatgctttcaatcgccttcgctgggaacagcgacttgggcctgcattacatcctctcaggctgcaagagcctgaagaaactggagatcagggactgcccatttggtgataagccattgctggcgaacgcagcaaagctggagacaatgcgatccctttggatgtcgtcgtgcttgttgaccctgggcgcatgccgacagcttgcacgcaagatgccccgccttagtgtggagatcatgaacgatcctggaaggtcatgccccttggattcgcttccggatgaaacacctgttgagaaactgtacgtctaccggacgatcgcaggtccaaggtccgacacaccagcctgcgtccagattgtgtag

SEQ ID NO: 11 - protein, 644 aa, Oryza sativa
MATTAAAAAATLSAAATAKTGRKNHQRHHVLPARGRVGAAAVRCSAVSPVTPPSPAPPATPLRPWGPAEPRKGADILVEALERCGVSDVFAYPGGASMEIHQALTRSPVITNHLFRHEQGEAFAASGYARASGRVGVCVATSGPGATNLVSALADALLDSVPMVAITGQVPRRMIGTDAFQETPIVEVTRSITKHNYLVLDVEDIPRVIQEAFFLASSGRPGPVLVDIPKDIQQQMAVPVWDTSMNLPGYIARLPKPPATELLEQVLRLVGESRRPILYVGGGCSASGDELRRFVELTGIPVTTTLMGLGNFPSDDPLSLRMLGMHGTVYANYAVDKADLLLAFGVRFDDRVTGKIEAFASRAKIVHIDIDPAEIGKNKQPHVSICADVKLALQGLNALLDQSTTKTSSDFSAWHNELDQQKREFPLGYKTFGEEIPPQYAIQVLDELTKGEAIIATGVGQHQMWAAQYYTYKRPRQWLSSAGLGAMGFGLPAAAGASVANPGVTVVDIDGDGSFLMNIQELALIRIENLPVKVMVLNNQHLGMVVQWEDRFYKANRAHTYLGNPECESEIYPDFVTIAKGFNIPAVRVTKKSEVRAAIKKMLETPGPYLLDIIVPHQEHVLPMIPNGGAFKDMILDGDGRTMY

SEQ ID NO: 12 - DNA, 1935 bp, Oryza sativa
atggctacgaccgccgcggccgcggccgccaccttgtccgccgccgcgacggccaagaccggccgtaagaaccaccagcgacaccacgtccttcccgctcgaggccgggtgggggcggcggcggtcaggtgctcggcggtgtccccggtcaccccgccgtccccggcgccgccggccacgccgctccggccgtgggggccggccgagccccgcaagggcgcggacatcctcgtggaggcgctggagcggtgcggcgtcagcgacgtgttcgcctacccgggcggcgcgtccatggagatccaccaggcgctgacgcgctccccggtcatcaccaaccacctcttccgccacgagcagggcgaggcgttcgcggcgtccgggtacgcgcgcgcgtccggccgcgtcggggtctgcgtcgccacctccggccccggggcaaccaacctcgtgtccgcgctcgccgacgcgctgctcgactccgtcccgatggtcgccatcacgggccaggtcccccgccgcatgatcggcaccgacgccttccaggagacgcccatagtcgaggtcacccgctccatcaccaagcacaattaccttgtccttgatgtggaggacatcccccgcgtcatacaggaagccttcttcctcgcgtcctcgggccgtcctggcccggtgctggtcgacatccccaaggacatccagcagcagatggctgtgccagtctgggacacctcgatgaatctaccggggtacattgcacgcctgcccaagccacccgcgacagaattgcttgagcaggtcttgcgtctggttggcgagtcacggcgcccgattctctatgtcggtggtggctgctctgcatctggtgatgaattgcgccggtttgttgagctgaccggcatcccagttacaaccactctgatgggcctcggcaatttccccagtgatgatccgttgtccctgcgcatgcttgggatgcatggcacggtgtacgcaaattatgcggtggataaggctgacctgttgcttgcatttggcgtgcggtttgatgatcgtgtgacagggaaaattgaggcttttgcaagcagggccaagattgtgcacattgacattgatccagcggagattggaaagaacaagcaaccacatgtgtcaatttgcgcagatgttaagcttgctttacagggcttgaatgctctgctagaccagagcacaacaaagacaagttctgattttagtgcatggcacaatgagttggaccagcagaagagggagtttcctctggggtacaagacttttggtgaagagatcccaccgcaatatgctattcaggtgctggatgagctgacgaaaggggaggcaatcatcgctactggtgttggacagcaccagatgtgggcggcacaatattacacctacaagcggccacggcagtggctgtcttcggctggtctgggcgcaatgggatttgggctgcctgctgcagctggtgcttctgtggctaacccaggtgtcacagttgttgatattgatggggatggtagcttcctcatgaacattcaggagttggcattgatccgcattgagaacctcccggtgaaggtgatggtgttgaacaaccaacatttgggtatggttgtgcaatgggaggataggttttacaaggcaaatagggcgcatacatacttgggcaacccagaatgtgagagcgagatatatccagattttgtgactattgctaaagggttcaatattcctgcagtccgtgtaacaaagaagagtgaagtccgtgccgccatcaagaagatgctcgagaccccagggccatacttgttggatatcatcgtcccacaccaggagcatgtgctgcctatgatcccaaatgggggcgcattcaaggacatgatcctggatggtgatggcaggactatgtattaa

SEQ ID NO: 13 - protein, 2327 aa, Oryza sativa
MTSTHVATLGVGAQAPPRHQKKSAGTAFVSSGSSRPSYRKNGQRTRSLREESNGGVSDSKKLNHSIRQGLAGIIDLPNDAASEVDISHGSEDPRGPTVPGSYQMNGIINETHNGRHASVSKVVEFCTALGGKTPIHSVLVANNGMAAAKFMRSVRTWANDTFGSEKAIQLIAMATPEDLRINAEHIRIADQFVEVPGGTNNNNYANVQLIVEIAERTGVSAVWPGWGHASENPELPDALTAKGIVFLGPPASSMHALGDKVGSALIAQAAGVPTLAWSGSHVEVPLECCLDSIPDEMYRKACVTTTEEAVASCQVVGYPAMIKASWGGGGKGIRKVHNDDEVRTLFKQVQGEVPGSPIFIMRLAAQSRHLEVQLLCDQYGNVAALHSRDCSVQRRHQKIIEEGPVTVAPRETVKELEQAARRLAKAVGYVGAATVEYLYSMETGEYYFLELNPRLQVEHPVTEWIAEVNLPAAQVAVGMGIPLWQIPEIRRFYGMNHGGGYDLWRKTAALATPFNFDEVDSKWPKGHCVAVRITSEDPDDGFKPTGGKVKEISFKSKPNVWAYFSVKSGGGIHEFADSQFGHVFAYGTTRSAAITTMALALKEVQIRGEIHSNVDYTVDLLNASDFRENKIHTGWLDTRIAMRVQAERPPWYISVVGGALYKTVTANTATVSDYVGYLTKGQIPPKHISLVYTTVALNIDGKKYTIDTVRSGHGSYRLRMNGSTVDANVQILCDGGLLMQLDGNSHVIYAEEEASGTRLLIDGKTCMLQNDHDPSKLLAETPCKLLRFLVADGAHVDADVPYAEVEVMKMCMPLLSPASGVIHVVMSEGQAMQAGDLIARLDLDDPSAVKRAEPFEDTFPQMGLPIAASGQVHKLCAASLNACRMILAGYEHDIDKVVPELVYCLDTPELPFLQWEELMSVLATRLPRNLKSELEGKYEEYKVKFDSGIINDFPANMLRVIIEENLACGSEKEKATNERLVEPLMSLLKSYEGGRESHAHFVVKSLFEEYLYVEELFSDGIQSDVIERLRLQHSKDLQKVVDIVLSHQSVRNKTKLILKLMESLVYPNPAAYRDQLIRFSSLNHKAYYKLALKASELLEQTKLSELRARIARSLSELEMFTEESKGLSMHKREIAIKESMEDLVTAPLPVEDALISLFDCSDTTVQQRVIETYIARLYQPHLVKDSIKMKWIESGVIALWEFPEGHFDARNGGAVLGDKRWGAMVIVKSLESLSMAIRFALKETSHYTSSEGNMMHIALLGADNKMHIIQESGDDADRIAKLPLILKDNVTDLHASGVKTISFIVQRDEARMTMRRTFLWSDEKLSYEEEPILRHVEPPLSALLELDKLKVKGYNEMKYTPSRDRQWHIYTLRNTENPKMLHRVFFRTLVRQPSVSNKFSSGQIGDMEVGSAEEPLSFTSTSILRSLMTAIEELELHAIRTGHSHMYLHVLKEQKLLDLVPVSGNTVLDVGQDEATAYSLLKEMAMKIHELVGARMHHLSVCQWEVKLKLDCDGPASGTWRIVTTNVTSHTCTVDIYREMEDKESRKLVYHPATPAAGPLHGVALNNPYQPLSVIDLKRCSARNNRTTYCYDFPLAFETAVRKSWSSSTSGASKGVENAQCYVKATELVFADKHGSWGTPLVQMDRPAGLNDIGMVAWTLKMSTPEFPSGREIIVVANDITFRAGSFGPREDAFFEAVTNLACEKKLPLIYLAANSGARIGIADEVKSCFRVGWSDDGSPERGFQYIYLSEEDYARIGTSVIAHKMQLDSGEIRWVIDSVVGKEDGLGVENIHGSAAIASAYSRAYKETFTLTFVTGRTVGIGAYLARLGIRCIQRLDQPIILTGYSALNKLLGREVYSSHMQLGGPKIMATNGVVHLTVSDDLEGVSNILRWLSYVPAYIGGPLPVTTPLDPPDRPVAYIPENSCDPRAAIRGVDDSQGKWLGGMFDKDSFVETFEGWAKTVVTGRAKLGGIPVGVIAVETQTMMQTIPADPGQLDSREQSVPRAGQVWFPDSATKTAQALLDFNREGLPLFILANWRGFSGGQRDLFEGILQAGSTIVENLRTYNQPAFVYIPMAAELRGGAWVVVDSKINPDRIECYAERTAKGNVLEPQGLIEIKFRSEELQDCMSRLDPTLIDLKAKLEVANKNGSADTKSLQENIEARTKQLMPLYTQIAIRFAELHDTSLRMAAKGVIKKVVDWEESRSFFYKRLRRRISEDVLAKEIRAVAGEQFSHQPAIELIKKWYSASHAAEWDDDDAFVAWMDNPENYKDYIQYLKAQRVSQSLSSLSDSSSDLQALPQGLSMLLDKMDPSRRAQLVEEIRKVLG

SEQ ID NO: 14 - DNA, 11927 bp, Oryza sativa
atgacatccacacatgtggcgacattgggagttggtgcccaggcacctcctcgtcaccagaaaaagtcagctggcactgcatttgtatcatctgggtcatcaagaccctcataccgaaagaatggtcagcgtactcggtcacttagggaagaaagcaatggaggagtgtctgattccaaaaagcttaaccactctattcgccaaggtgaccactagctactttacatatgctataatttgtgccaaacataaacatgcaatggctgctattatttaaacgttaatgttgaaatagctgctataggatacagcaaaaatatataattgactgggcaagatgcaacaattgtttttcactaaagttagttatcttttgctgtaaaagacaactgttttttacataaaatggtattaataaccttgtaatattcaatgcaacatgttctcaagtaaaaaaaaacattgcctggttgtataagcaaatgtgtcgttgtagacatcttattaaacctttttgtgatatctattaccgtagggaacaggggagctgtttaaatctgttatcatagagtaatatgagaaaagtggattgtgcgactttggcatgtatacctgctcaatttcaaatatatgtctatgtgcaggtcttgctggcatcattgacctcccaaatgacgcagcttcagaagttgatatttcacagtaaggactttatattttataataattattatataattttctgacatgttttgagaacctcaaaacatgtgattgcaccttccttttttatgtctggttcagaaactgataagttttgacagtgtttaggatggatctttgatgcgcacagtgctttctaatgttttcatttttgaaagtaatgttttaggaagaaatatctgattaaatttatactttatctttacaaaagtcaaatgcgttctgtatcaattgcggtttgtaatatggcaagaacatgctttcagaatttgttcatacaatgctttctttctattattatgtagaacaaatacctaatactttgttcaccttttatagtggacacctctcacagctttttcagtaagtgatgcaattttgtacatttgtaagatgtgttccagaaaccttttctcctgcaattctaatgtacccactcaaactggtatcaccaaagatctccatctgattgaaaaaaagctgcgtgaagtatgcttatttatgctaaccatacatgatttatactgttttatagtacaatgcttatttatgctaaccatacataattttattctgttttctagtacattatttgtgcccctgaccataaatgatcctttcttttacagtggttccgaagatcccagggggcctacggtcccaggttcctaccaaatgaatgggattatcaatgaaacacataatgggaggcatgcttcagtctccaaggttgttgagttttgtacggcacttggtggcaaaacaccaattcacagtgtattagtggccaacaatggaatggcagcagctaagttcatgcggagtgtccgaacatgggctaatgatacttttggatcagagaaggcaattcagctgatagctatggcaactccggaggatctgaggataaatgcagagcacatcagaattgccgatcaatttgtagaggtacctggtggaacaaacaacaacaactatgcaaatgtccaactcatagtggaggttagttcagctcatccctcaacacaacattttcgtttctatttaagttagggaaaaatctctacgaccctccaatttctgaacatccaattttcaccatcaactgcaatcacagatagcagagagaacaggtgtttctgctgtttggcctggttggggtcatgcatctgagaatcctgaacttccagatgcgctgactgcaaaaggaattgtttttcttgggccaccagcatcatcaatgcatgcattaggagacaaggttggctcagctctcattgctcaagcagctggagttccaacacttgcttggagtggatcacatgtgagccttgtcttctcttttttagcttatcatcttatcttttcggtgatgcattatcccaatgacactaaaccataggtggaagttcctctggagtgttgcttggactcaatacctgatgagatgtatagaaaagcttgtgttactaccacagaggaagcagttgcaagttgtcaggtggttggttatcctgccatgattaaggcatcttggggtggtggtggtaaaggaataaggaaggtttgttcttcttgtagttatcaagagattgtttggattgcaagtgtttagtgcccatagttaactctggtctttctaacatgagtaactcaactttcttgcaggttcataatgatgatgaggttaggacattatttaagcaagttcaaggcgaagtacctggttccccaatatttatcatgaggctagctgctcaggtggggccttttatggaagttacaccttttcccttaatgttgagttattccggagttattatggttatgttctgtatgtttgatctgtaaattattgaaattcacctccattggttctccagattagcagacctacaattctacatatggtttatactttataaatactaggatttagggatcttcatatagtttatacatggtatttagatttcatttgtaaccctattgaagacatcctgattgttgtcttatgtagagtcgacatcttgaagttcagttgctttgtgatcaatatggcaacgtagcagcacttcacagtcgagattgcagtgtacaacggcgacaccaaaaggtctgctgtctcagttaaatcacccctctgaatgatctacttcttgcctgctgcgttggtcagaggaataatggttgtattctactgaacagataatcgaggaaggaccagttactgttgctcctcgtgagactgtgaaagagcttgagcaggcagcacggaggcttgctaaagctgtgggttatgttggtgctgctactgttgaatacctttacagcatggaaactggtgaatattattttctggaacttaatccacggctacaggtcggctcctttgacattcttcaggaattaatttctgttgaccacatgatttacattgtcaaatggtctcacaggttgagcatcctgtcactgagtggatagctgaagtaaatttgcctgcggctcaagttgctgttggaatgggtatacccctttggcagattccaggtaatgcttcttcatttagttcctgctctttgttaattgaatgagctcttatacagaccatgagacacattctactgttaattcatagtatcccctgacttgttagtgttagagatacagagatgtatcacaaattcattgtatctcctcaaggactgtaaaaatcctataattaaatttctgaaaatttgttcttttaagcagaaaaaaaatctctaaattatctccctgtatacagagatcaggcgcttctacggaatgaaccatggaggaggctatgacctttggaggaaaacagcagctctagcgactccatttaactttgatgaagtagattctaaatggccaaaaggccactgcgtagctgttagaataactagcgaggatccagatgatgggtttaagcctactggtggaaaagtaaaggtgcggtttcctgatgttaggtgtatgaattgaacacattgctatattgcagctagtgaaatgactggatcatggttctcttattttcaggagataagtttcaagagtaaaccaaatgtttgggcctatttctcagtaaaggtagtcctcaatattgttgcactgccacattatttgagttgtcctaacaattgtgctgcaattgttagttttcaactatttgttgttctgtttggttgactggtaccctctctttgcagtctggtggaggcatccatgaattcgctgattctcagttcggtatgtaaagttaaaagagtaatattgtctttgctatttatgtttgtcctcacttttaaaagatattgccttccattacaggacatgtttttgcgtatggaactactagatcggcagcaataactaccatggctcttgcactaaaagaggttcaaattcgtggagaaattcattcaaacgtagactacacagttgacctattaaatgtaaggactaaatatctgcttattgaaccttgctttttggttccctaatgccattttagtctggctactgaagaacttatccatcatgccatttctgttatcttaaattcaggcctcagattttagagaaaataagattcatactggttggctggataccaggatagccatgcgtgttcaagctgagaggcctccatggtatatttcagtcgttggaggggctttatatgtaagacaaactatgccactcattagcatttatgtgaagcaaatgcggaaaacatgatcaatatgtcgtcttatttaaatttatttatttttgtgctgcagaaaacagtaactgccaacacggccactgtttctgattatgttggttatcttaccaagggccagattccaccaaaggtactattctgttttttcaggatatgaatgctgtttgaatgtgaaaaccattgaccataaatccttgtttgcagcatatatcccttgtctatacgactgttgctttgaatatagatgggaaaaaatatacagtaagtgtgacattcttaatggggaaacttaatttgttgtaaataatcaatatcatattgactcgtgtatgctgcatcatagatcgatactgtgaggagtggacatggtagctacagattgcgaatgaatggatcaacggttgacgcaaatgtacaaatattatgtgatggtgggcttttaatgcaggtaatatcttcttcctagttaaagaagatatatcttgttcaaagaattctgattattgatcttttaatgttttcagctggatggaaacagccatgtaatttatgctgaagaagaggccagtggtacacgacttcttattgatggaaagacatgcatgttacaggtaatgatagccttgttctttttagttctagtcacggtgtttgcttgctatttgttgtatctatttaatgcattcactaattactatattagtttgcatcatcaagttaaaatggaacttctttcttgcagaatgaccatgacccatcaaagttattagctgagacaccatgcaaacttcttcgtttcttggttgctgatggtgctcatgttgatgctgatgtaccatatgcggaagttgaggttatgaagatgtgcatgcccctcttatcacccgcttctggtgtcatacatgttgtaatgtctgagggccaagcaatgcaggtacattcctacattccattcattgtgctgtgctgacatgaacatttcaagtaaatacctgtaacttgtttattattctaggctggtgatcttatagctaggctggatcttgatgacccttctgctgttaagagagctgagccgttcgaagatacttttccacaaatgggtctccctattgctgcttctggccaagttcacaaattatgtgctgcaagtctgaatgcttgtcgaatgatccttgcggggtatgagcatgatattgacaaggtaaacatcatgtcctcttgttttttcttttgtttatcatgcattcttatgttcatcatgtcctctggcaaatctagattccgctgtcgtttcacacagatttttctcattctcataatggtgccaaacataaatatgctgctatattcatcaatgttttcactcgatttctaattttgcttttgagttttaaactttagtacaatccatatctaatctcctttggcaacagtgaatccattatatatatttttattaaactgctttctttttcaggttgtgccagagttggtatactgcctagacactccggagcttcctttcctgcagtgggaggagcttatgtctgttttagcaactagacttccaagaaatcttaaaagtgaggtatattatggttgacaagatagctagtctcatgctctaaggacttgtacatttcgccacataggttaattttccatatcaagttctaatgtacgatataaaagtagtactggcctaaaacagtattggtggttgactatctttgttgtgtaagatcaagtatttctttttcatgcttagtttgtcaatacttcacatttatcactgacttgtcgagctaaatgagattttatttgatttctgtgctccattatttttgtatatatatatatatatttaactatgactatatgttatgcctcaaacgtttcaaactctttcagttggagggcaaatatgaggaatacaaagtaaaatttgactctgggataatcaatgatttccctgccaatatgctacgagtgataattgaggtcagttattcaatttgttgtgataatcactgccttaactgttcgttcttttaacaagcggttttataggaaaatcttgcatgtggttctgagaaggagaaggctacaaatgagaggcttgttgagcctcttatgagcctactgaagtcatatgagggtgggagagaaagtcatgctcactttgttgtcaagtccctttttgaggagtatctctatgttgaagaattgttcagtgatggaattcaggttaacttacctattcgcattaaacaaatcatcagttgttttatgataaagtcaaaatgtttatatttcccattcttctgtggatcaaatatatcacggacatgatatagtttccttaggctatataatggttcttcatcaaataatattgcaggaaacagtatagcaaactatttgtatatactcgagatggaaattgttagaaacatcattgactaaatctgtcctttgttacgctgtttttgtagtctgatgtgattgagcgtctgcgccttcaacatagtaaagacctacagaaggtcgtagacattgtgttgtcccaccaggtaaatttcttcatggtctgatgacttcactgcgaatggttactgaactgtcttcttgttctgacaatgtgacttttctttgtagagtgttagaaataaaactaagctgatactaaaactcatggagagtctggtctatccaaatcctgctgcctacagggatcaattgattcgcttttcttcccttaatcacaaagcgtattacaaggtgaccaggataaacataaataaacgtgaatttttcaatgaccttttcttctgacatctgaatctgatgaatttcttgcatattaatacagttggcacttaaagctagtgaacttcttgaacaaacaaaacttagtgagctccgtgcaagaatagcaaggagcctttcagagctggagatgtttactgaggaaagcaagggtctctccatgcataagcgagaaattgccattaaggagagcatggaagatttagtcactgctccactgccagttgaagatgcgctcatttctttatttgattgtagtgatacaactgttcaacagagagtgattgagacttatatagctcgattataccaggtatgagaagaaagaccttttgaaattatttatattaacatatcctagtaaaacagcatgctcatcatttcttaaaaaaagtttacagcacctgatgtttggttactgaccgcatcattaaaataaagttacttgttgtggagagatgtattttggaacttgtggcacatgcagtaacatgctactgctcgatatgtttgctaacttgacaacaatatttttcagcctcatcttgtaaaggacagtatcaaaatgaaatggatagaatcgggtgttattgctttatgggaatttcctgaagggcattttgatgcaagaaatggaggagcggttcttggtgacaaaagatggggtgccatggtcattgtcaagtctcttgaatcactttcaatggccattagatttgcactaaaggagacatcacactacactagctctgagggcaatatgatgcatattgctttgttgggtgctgataataagatgcatataattcaagaaaggtatgttcatatgctatgttggtgctgaaatagttatatatgtagttagctggtggagttctggtaattaacctatcccattgttcagtggtgatgatgctgacagaatagccaaacttcccttgatactaaaggataatgtaaccgatctgcatgcctctggtgtgaaaacaataagtttcattgttcaaagagatgaagcacggatgacaatgcgtcgtaccttcctttggtctgatgaaaagctttcttatgaggaagagccaattctccggcatgtggaacctcctctttctgcacttcttgagttggtacgtgatatcatcaaaatgataatgttttggtatggcattgattatcttctatgctctttgtatttattcagcctattgtggatacaggacaagttgaaagtgaaaggatacaatgaaatgaagtataccccatcacgggatcgtcaatggcatatctacacacttagaaatactgaaaaccccaaaatgttgcaccgggtatttttccgaacccttgtcaggcaacccagtgtatccaacaagttttcttcgggccagattggtgacatggaagttgggagtgctgaagaacctctgtcatttacatcaaccagcatattaagatctttgatgactgctatagaggaattggagcttcacgcaattagaactggccattcacacatgtatttgcatgtattgaaagaacaaaagcttcttgatcttgttccagtttcagggtaagtgcgcatatttctttttgggaacatatgcttgcttatgaggttggtcttctcaatgatcttcttatcttactcaggaatacagttttggatgttggtcaagatgaagctactgcatattcacttttaaaagaaatggctatgaagatacatgaacttgttggtgcaagaatgcaccatctttctgtatgccaatgggaagtgaaacttaagttggactgcgatggtcctgccagtggtacctggaggattgtaacaaccaatgttactagtcacacttgcactgtggatgtaagtttaatcctctagcattttgttttctttggaaaagcatgtgattttaagccggctggtcctcatacccagacctagtgatctttatatagtgtagacatttttctaactgcttttaattgttttagatctaccgtgagatggaagataaagaatcacggaagttagtataccatcccgccactccggcggctggtcctctgcatggtgtggcactgaataatccatatcagcctttgagtgtcattgatctcaaacgctgttctgctaggaataatagaactacatactgctatgattttccactggtgagttgactgctcccttatattcaatgcattaccatagcaaattcatattcgttcatgttgtcaaaataagccgatgaaaattcaaaactgtaggcatttgaaactgcagtgaggaagtcatggtcctctagtacctctggtgcttctaaaggtgttgaaaatgcccaatgttatgttaaagctacagagttggtatttgcggacaaacatgggtcatggggcactcctttagttcaaatggaccggcctgctgggctcaatgacattggtatggtagcttggaccttgaagatgtccactcctgaatttcctagtggtagggagattattgttgttgcaaatgatattacgttcagagctggatcatttggcccaagggaagatgcattttttgaagctgttaccaacctagcctgtgagaagaaacttcctcttatttatttggcagcaaattctggtgctcgaattggcatagcagatgaagtgaaatcttgcttccgtgttgggtggtctgatgatggcagccctgaacgtgggtttcagtacatttatctaagcgaagaagactatgctcgtattggcacttctgtcatagcacataagatgcagctagacagtggtgaaattaggtgggttattgattctgttgtgggcaaggaagatggacttggtgtggagaatatacatggaagtgctgctattgccagtgcttattctagggcatataaggagacatttacacttacatttgtgactggaagaactgttggaataggagcttatcttgctcgacttggcatccggtgcatacagcgtcttgaccagcctattattcttacaggctattctgcactgaacaagcttcttgggcgggaagtgtacagctcccacatgcagttgggtggtcccaaaatcatggcaactaatggtgttgtccatcttactgtttcagatgaccttgaaggcgtttctaatatattgaggtggctcagttatgttcctgcctacattggtggaccacttccagtaacaacaccgttggacccaccggacagacctgttgcatacattcctgagaactcgtgtgatcctcgagcggctatccgtggtgttgatgacagccaagggaaatggttaggtggtatgtttgataaagacagctttgtggaaacatttgaaggttgggctaagacagtggttactggcagagcaaagcttggtggaattccagtgggtgtgatagctgtggagactcagaccatgatgcaaactatccctgctgaccctggtcagcttgattcccgtgagcaatctgttcctcgtgctggacaagtgtggtttccagattctgcaaccaagactgcgcaggcattgctggacttcaaccgtgaaggattacctctgttcatcctcgctaactggagaggcttctctggtggacaaagagatctttttgaaggaattcttcaggctggctcgactattgttgagaaccttaggacatacaatcagcctgcctttgtctacattcccatggctgcagagctacgaggaggggcttgggttgtggttgatagcaagataaacccagaccgcattgagtgctatgctgagaggactgcaaaaggcaatgttctggaaccgcaagggttaattgagatcaagttcaggtcagaggaactccaggattgcatgagtcggcttgacccaacattaattgatctgaaagcaaaactcgaagtagcaaataaaaatggaagtgctgacacaaaatcgcttcaagaaaatatagaagctcgaacaaaacagttgatgcctctatatactcagattgcgatacggtttgctgaattgcatgatacatccctcagaatggctgcgaaaggtgtgattaagaaagttgtggactgggaagaatcacgatctttcttctataagagattacggaggaggatctctgaggatgttcttgcaaaagaaattagagctgtagcaggtgagcagttttcccaccaaccagcaatcgagctgatcaagaaatggtattcagcttcacatgcagctgaatgggatgatgacgatgcttttgttgcttggatggataaccctgaaaactacaaggattatattcaatatcttaaggctcaaagagtatcccaatccctctcaagtctttcagattccagctcagatttgcaagccctgccacagggtctttccatgttactagataaggtaattagcttactgatgcttatataaattctttttcattacatatggctggagaactatctaatcaaataatgattataattccaatcgttctttttatgccattatgatcttctgaaatttccttctttggacacttattcagatggatccctctagaagagctcaacttgttgaagaaatcaggaaggtccttggttga

SEQ ID NO: 15 - DNA, 4203 bp, Artificial Sequence
  source: /note=“Description of Artificial Sequence Synthetic polynucleotide”
atggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtga

SEQ ID NO: 16 - DNA, 4203 bp, Artificial Sequence
  source: /note=“Description of Artificial Sequence Synthetic polynucleotide”
atggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgtcagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagcagtaccggagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtga

SEQ ID NO: 17 - DNA, 5860 bp, Oryza sativa
atggccacggcgatcccgcagcggcagctcttcgtcgccggcgagtggcgcgcccccgcgctcggccgccgcctccccgtcgtcaaccccgccaccgagtcccccatcggtaccctcctcttcaccctctccaccctctgcttctgcctctgattagcctttttgttgttgttgttgttgctgctgttttttgcgtgtcggtgcgcaggcgagatcccggcgggcacggcggaggacgtggacgcggcggtggcggcggcgcgggaggcgctgaagaggaaccggggccgcgactgggcgcgcgcgccgggcgccgtccgggccaagtacctccgcgcaatcgcggccaaggtagggtggtgactacccccacccccccccccccccccaacgcgacccgcgtgcgtgtgttccgtacagggggaggagctccgcgtggctctccagtaggtttttgagccccaaatcgatcgatatgctctagttttaagtttgctgcttaaattcctcaagggtttagtttgcaaccaaatccttattttagcttcggtataagccccccatatgatgtgcgtgcgtcggcatcggaagtgcgtatcctctgttctggactaggaattggccataggttgatcgacagttcgagtattctgcttctgtttggaataagttggaagcatggctgattgtgtatctggatgctgtttttgtggtgattcgtttcaagctcttgttaattgatgggttcaagcggagagggtgcgcaacaacaagtgtatatggctcacggccatgggtgtgcacatttgattggtgcgcaacaacaagtgtatattgtttgtgtgcttcgttagttggcaggtcctagtcactaaatcactattggattggtactagttacttttgtgccttgacgatgggactggattactagccttttggttgcctttgtggtattccgttgttatgggcctgttgatggatggatccctttaatttctagtgccaaatgcatgctagatttctcacagtttttctcttcaggttatatttctcgtatttccttttcctaaaggattgctttttcatgtattttctggcatatataggttattattattattattctccagaacaagattacccatattatggatcactagtgtacacttttttggatgaaaaacctacttactgaaagtaaaacagtgaccagtgcacactttacttgaactgtcaaaccatcaattttctagcaaagcaggggatgctagccttccagtctaaatgacagtaaactactatacttttgtccgtaggtttggaaatatgctaatttctatcataaaaattttcatggcatatgcgagcattttatgatcaccttttccctttttcttcagataatcgagaggaaatctgagctggctagactagagacgcttgattgtgggaagcctcttgatgaagcagcatgggacatggtatgtggccagttatccactgtatgaatatgtagttgcctacacagcaatctttcctgaacatgaatcctgatgtatgatattccatttgtcaggacgatgttgctggatgctttgagtactttgcagatcttgcagaatccttggacaaaaggcaaaatgcacctgtctctcttccaatggaaaactttaaatgctatcttcggaaagagcctatcggtgtagttgggttgatcacaccttggtatttcacatttttctctcatcctgcgcttatatttatttatgacccaagcatggtactaaatagtactagtaacatgcatatactgaatgagtttacaactttacatgatttttttgaactatgaaagttgaagacatttgagattttattcctcttctcttgtgcaaacatattattgtctcacaaattgtacctagcagctactctctccgtttcatattataagtcgtttgacttttttcctagtcaaaatgtgttaagtttgaccaagtttatagaaaaatttagcaacatctaaaatatcaaagtcatgttttagtgttttttcaggctctcatgtaagcaattttgatgtgccctctcctttcttcttaatataatgatacacagctcttgtgtattcaaaggaaaatatatatatatataatgatacacacctctcctccgtgttaatgcagctcatttgttctgtcccggttcaaatatctatttttctcatatgttgtcagcatgattcacttaatttagtatatagaagatgccattatttatgtctggaatcttactgcagaagggaaaacaattgataacggaattgattgcattctaatttgttgtttctttgttatgttcttatcgacaattacaaatttgattctgagaatcatgttcgggatgtgtatttctactgcaggaactatcctctcctgatggcaacatggaaggtagctcctgccctggctgctggctgtacagctgtactaaaaccatctgaattggcttccgtgtaagtttaacatgttaacttgttaatgtcatacccatgctagttgcaatgacatttgattttaaaatgttgtggcatgtccatgctgcaagcaatgtaatttgaaatctctctctatcattaattaccaggacttgtttggagcttgctgatgtgtgtaaagaggttggtcttccttcaggtgtgctaaacatagtgactggattaggttctgaagccggtgctcctttgtcatcacaccctggtgtagacaaggtacagctattcctcctgtaatcatgtataccccatcaatggaaatgatattcctctcaatacatggtttatgttttctgttaggttgcatttactgggagttatgaaactggtaaaaagattatggcttcagctgctcctatggttaaggtttgtttccaaatttctgtggatattttttgttctctttctactaactctctattatcaattctcaatgttgtccttttcttttaactcctttactttttagaattgtgatcaagacactttgagcatcattctagtagccagttctatcctgtttcttacctttttatggttcgtcttttcttgacagcctgtttcactggaacttggtggaaaaagtcctatagtggtgtttgatgatgttgatgttgaaaaaggtacatgccacttgctatgattaactaattctgaagtgcgggactttgtaaagcacttaactgagctggatgctagacccccaaaagccctttttggtgtcttgggcttgttgcagaaatactggtcccagacgagcaggatgcaagaaaattaactacttttgccactgattagtatttcttagaagttacacctcaaggattagcaatactttcttaaaatgtgctattgattaaaaagatgtcctgtattattttgagcagatcttgtactggttgatcggcttgcatgaaaatattgttgaggattataatgccatgccaactgagtaaagaaaagagttgtaaaatatgttatgcaacatgaatatatatgtgatttcatttttcctttttcttttcgtggcaaggaaggcagttaggaaggactgatgtgaaaagcacaagtactattcttagttctggaaaactgtgttctttattttcctaactacaattcaccttgattagtcagtaacttgatattggcaattctagctgattatgaattctgtttatatttcactaattttgaatctttaattacattttatggttgaaatttaacgttttgtctggttatggactctgtttgtattcactcaatttggatcttccattagatttcattgttggtccttcttcttgtacagctgttgagtggactctctttggttgcttttggaccaatggccagatttgcagtgcaacatcgcgtcttattcttcatgtaagcattgaatatatccgtcaatcataatctattgttgtacttgattttttttctgatcaactcctgagttcagattattatatgatgccattactattgcacagagcgaataaaattgtatttatgcacagcatgtattttgagtaatatatgcattgcctattatttaatatatagattgtagcacttaattttgtgtccatgtctctatgatgtttattactttattattgccggcatgaagcaactttgaactctatgttgatcttgaactaaaattgaaattaattggcttattgctattaatgatatagctttcagcttcttgctcctgaccatgaaagttttgcagaaaaaaatcgctaaagaatttcaagaaaggatggttgcatgggccaaaaatattaaggtgtcagatccacttgaagagggttgcaggcttgggcccgttgttagtgaaggacaggtaccacatgtaaactttttctaaattcaaaaaagaaatgccactgatcaatggtaggtccttccaagccttattgctggattgttgcactgttttgtcaattttgtgtaatatagttctgaatgaattagtcggtgtatgctcttgctagttgctagtatgtggtacagggtcttcctactttgagcaaattcgtgttaaaatgcattgatgaaaaggccacctttccgtaggtttatcttgtcataatttaaaccccaataaaattttaattttttgttttgaccccatggcactttaatgaaatcacttagccatgagcttttgtatatattttcaaagcaccagaatgtttagatggtttgttggaaatcttacacatcctattgccttgtgtcagtatgagaagattaagcaatttgtatctaccgccaaaagccaaggtgctaccattctgactggtggggttagacccaaggtaataatctactacacggttgtatatataggtacccacatatcattatgaagtagaaataatcttgtatgtttttgtcagcatctggagaaaggtttctatattgaacccacaatcattactgatgtcgatacatcaatgcaaatttggagggaagaagtttttggtccagtgctctgtgtgaaagaatttagcactgaagaagaagccattgaattggccaacgatactcagtgagttttttttttaatacagttcattgtcctgttcaatcttgcagcatatgtatatactctgtggcatatgaacttattctgctactactacttttgatagttatggtctggctggtgctgtgctttccggtgaccgcgagcgatgccagagattaactgaggtatatccaagtgaagggggttggcattgtttgattcatatgacatggttgcatcaagctgatattcaagaatctcatttattacttgcattctatgcatctccagttcttccctggactccggtcaatgttaatatagtttgtttgctagtagtatgctactccaattaagttgctcttcacttccacatcatctgatccatgactttatatttgaccccttttttttgcaaaagaaagggaaatacttaacgaaaatttcctactgcaggagatcgatgccggaattatctgggtgaactgctcgcaaccctgcttctgccaagctccatggggcgggaacaagcgcagcggctttggacgcgagctcggagaagggtgggtagcacacaacaatctcactttaaaacaccatttcgatcgtctgatgatctcgacctgacatcatgcctttggtattttcattcacttttcaggggcattgacaactacctaagcgtcaagcaagtgacggagtacgcctccgatgagccgtggggatggtacaaatccccttccaagctgtaa

SEQ ID NO: 18 - DNA, 2192 bp, Oryza sativa
atggctggcatgtctcttcagcatccctgggccttcgcctttggtctcctaggtgtgttgcctttgatctgatccaaggaattctcttgagaattaatcttgcatggttatttacttttgttgttattattctctacatttttaatcatgtacttttccatgttccacttttgttgccaaagctactatatttttcctaccaattcatccaaaactactatattatagcaaggcagctagtggatcgactttgcacttttggatgcaattgtgagtggtttacattagaggggccatgcatagccaaataaattgtttggcaaaatatgatttcccttgtagttagcacaatattgtgatctttcattcctttactctttttgttccaccaccctcattattgcaaagccccctcttttagaaccaaactaaagataagaaacatttgattcatcattagaggagattagatatacatacatactacacatgcattgctgtgataagcctagccaggcacagaaagagccaagacaaaaggagctcaaaagaaaaggggctccaatatcttgcccggcctgatcagtgtaacatgacgtcacccacacaattattaaaaggatagatgcatatgtgttcgttacgtatctccttgtgtcacgttgctgtgcatggttcactcgcttaattataacgtccgtcgcattttatgcatgcacttcgtttttagttaatcaaattaaacaagatcctaacatatgatttctattttattctctttgcaggcaacatcatctccttcatgacctacctggccccactgtaagtcccacacatccggatattttcttttctttagcgaaattttacattgctccagaaagtaccttttacactaaaatttcttatacaccaccataaaattaatgtggtactattggtaccttatcaaaaatgataaaattacctttttcttcagataaaaacactccccgtctctgtataactaagatgtagacaaccaatacacatgtctagagctaatcctatcaattaacattgctagtaaatacaaggaaaaatataaagaattaaccaattgcttggatgtcttgcaggccgacgttctacaggatctacaagagcaagtcgacgcaggggttccagtcggtaccctacgtggtggcgctgttcagcgcgatgctgtggatctactacgcgctgctcaagtccgacgagtgcctcctcatcaccatcaactccgctggctgcgtcatcgagaccatctacatcgccgtctacctcgtctacgcccccaagaaggccaagatgttcaccgccaagctcctcctcctcgtcaacgtcggcgtcttcggcctcatcctcctcctcaccctcctcctctccgccggcgaccgccgcatcgtggttcttggttgggtctgcgttggcttctccgtcagcgtcttcgtcgccccccttagcatcatcgtaagaaaccctagaattgcctgtaaacaataataactttcggtctctgcaccaactaaggatgcacatatttacgcaccgcttttgctatctctgcagaggctggtggtgcgcaccaagagcgtggagttcatgccgttctcgctctccttctccctcaccatcagcgccgtcgtctggttcctctacggcctcctcatcaaggacaaatatgtcgctgtgagtagctccgattcgacccgttcttcttcctaaattttccgctgctcgatttaatttctatttctaattgtggaagattgtttaattatagtgattatggctagctgataaccgattttaatttctgatggtgatccaaaatggaacagcttcccaacgtgctgggcttctccttcggcgtcatccagatggggctgtacgccatgtacaggaactcgacgcccaaggccgtgctgaccaaggaggtcgaggcggcgacggccaccggcgacgacgaccactccgccgccggcgtcaaggagcacgtcgtcaacatcgccaagctctctgccgccgtcgacgtcgtcaagacccgcgaggtgcaccccgtcgacgtcgagtccccgccggcagaggcgccgcctgaggaggacgacaaggccgccgccgccaccgccgccgccgtcgccggcgccggcgagaagaaggtagctgcatga

SEQ ID NO: 19 - protein, 657 aa, Solanum tuberosum
MAAASPSPCFSKNLPPSSSKSSILLPKSTFTFHNHPKNTSPLHLTHTQHHSRFTVSNVILSTTTHNDVSEPEIFVSRFAPDEPRKGCDVLVEALEREGVKDVFAYPGGASMEIHQALTRSNIIRNVLPRHEQGGVFAAEGYARATGFPGVCIATSGPGATNLVSGLADALLDSIPIVAITGQVPRRMIGTDAFQETPIVEVTRSITKHNYLVMDVEDIPRVVREAFFLAKSGRPGPVLIDVPKDIQQQLVIPNWDQPMRLPGYISRLPKLPNEMLLEQIVRLISESKKPVLYVGGGCTQSSEELRRFVELTGIPVASTLMGLGTFPCGDELSLQMLGMHGTVYANYAVDSSDLLLAFGVRFDDRVTGKLEAFASRAKIVHIDIDSAEIGKNKQPHVSICADIKLALQGLNSILEGKEGKLKLDFSAWRQELTEQKVKYPLNYKTFGEAIPPQYAIQVLDELTNGNAIISTGVGQHQMWAAQYYKYKKPRQWLTSGGLGAMGFGLPAAIGAAVGRPGEIVVDIDGDGSFIMNVQELATIKVENLPVKIMLLNNQHLGMVVQWEDRFYKANRAHTYLGNPANEEEIFPNMLKFAEACGVPAARVSHRDDLRAAIQKMLDTPGPYLLDVIVPHQEHVLPMIPSGGAFKDVITEGDGRRSY

SEQ ID NO: 20 - DNA, 1974 bp, Solanum tuberosum
atggcggctgcatctccatctccttgtttttccaaaaacctacctccatcttcatcaaaatcttccatccttcttcccaaatctacctttactttccacaatcaccccaaaaatacctcaccccttcaccttacccacacccaacatcatagccgtttcactgtctcaaatgtcatcctatcaaccacgacccataacgacgtttctgaacccgaaatcttcgtttcacgtttcgcccctgacgaacccagaaagggttgtgatgttcttgtggaggcacttgaaagggaaggggttaaggatgtatttgcatacccaggaggtgcttccatggagattcatcaggctttgacacgttccaatattattcgtaatgtgctgccacgtcatgaacagggtggtgtgtttgctgcagagggttacgcacgggccactgggttccctggtgtttgcattgctacatctggtccgggagctacgaatcttgttagcggtcttgcggatgctttgttggatagtattccgattgttgctattacgggtcaagtgccgaggaggatgattggtactgatgcgtttcaggaaactcctattgttgaggtaacgagatccattacgaagcataattatcttgttatggatgtagaggatattcctagggttgttcgtgaagcgttttttctagcgaaatcgggacggcctggaccggttctgattgatgttcctaaggatattcagcaacaattggtgatacctaattgggatcagccaatgaggttgcctggttacatctctaggttgcctaaattgcctaatgagatgcttttggaacaaattgttaggctgatttcggagtcgaagaagcctgttttgtatgtgggtggtgggtgtacacaatcgagtgaggagctgagacgatttgtggagcttacgggtattcctgtggcgagtactttgatgggtcttggaacttttccatgtggggatgagctttctcttcaaatgttgggtatgcatgggactgtgtatgctaattatgcggtggatagtagtgatttgttgcttgcatttggggtgaggtttgatgatcgagttactggtaaattggaagcttttgctagccgagcgaaaattgtccacattgatattgattcggctgagattggaaagaacaagcaacctcatgtttccatttgtgcagatatcaagttggcattacagggtttgaattccatattggagggtaaagaaggtaagctgaagttggacttttctgcttggaggcaggagttaacggagcagaaggtgaagtacccattgaattataagacttttggtgaagccatccctccacaatatgctattcaggttcttgatgagttaactaacggaaatgccattattagtactggtgtggggcaacaccaaatgtgggctgcccaatactataagtacaaaaagccacgccaatggttgacatctggtggattaggagcaatgggatttggtttgcctgctgctataggtgcggctgttggaagaccgggtgagattgtggttgacattgatggtgacgggagttttatcatgaatgtgcaggagttagcaacaattaaggtggagaatctcccagttaagattatgttactgaataatcaacacttgggaatggtggttcagtgggaggatcgattctataaggctaacagagcacacacttacttgggtaatcctgctaatgaggaagaaatcttccctaatatgctgaaatttgcagaggcttgtggcgtacctgctgcaagagtgtcacacagggatgatcttagagctgccattcaaaagatgttagacactcctgggccatacttgttggatgtgattgtacctcatcaggagcacgttctacctatgattcccagtggtggcgctttcaaagatgtgattacggagggtgatgggagacgttcctattga

SEQ ID NO: 21 - DNA, 1424 bp, Homo sapiens
atggtgcacctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaa

SEQ ID NO: 22 - DNA, 444 bp, Homo sapiens
atggtgcacctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaa

SEQ ID NO: 23 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 24 - DNA, 19 bp, Artificial Sequence
  source: /note=“Description of Artificial Sequence Synthetic oligonucleotide”
gcatggttatgcaatggga

SEQ ID NO: 25 - DNA, 19 bp, Artificial Sequence
  source: /note=“Description of Artificial Sequence Synthetic oligonucleotide”
ggcatggttatgcaatgga

SEQ ID NO: 26 - DNA, 19 bp, Artificial Sequence
  source: /note=“Description of Artificial Sequence Synthetic oligonucleotide”
tgccgatgatcccgagtgg

SEQ ID NO: 27 - DNA, 19 bp, Artificial Sequence
  source: /note=“Description of Artificial Sequence Synthetic oligonucleotide”
ttgccgatgatcccgatgg

SEQ ID NO: 28 - DNA, 20 bp, Artificial Sequence
  source: /note=“Description of Artificial Sequence Synthetic primer”
attgacggagatggaagctt

SEQ ID NO: 29 - DNA, 20 bp, Artificial Sequence
  source: /note=“Description of Artificial Sequence Synthetic primer”
ccaaactgagccagtcacaa

SEQ ID NO: 30 - DNA, 54 bp, Artificial Sequence
  source: /note=“Description of Artificial Sequence Synthetic primer”
atatatggtctcgattggcatggttatgcaatgggagttttagagctagaaata

SEQ ID NO: 31 - DNA, 54 bp, Artificial Sequence
  source: /note=“Description of Artificial Sequence Synthetic primer”
attattggtctcgaaactccattgcataaccatgcccaatctcttagtcgactc

SEQ ID NO: 32 - DNA, 54 bp, Artificial Sequence
  source: /note=“Description of Artificial Sequence Synthetic primer”
atatatggtctcgattgtgccgatgatcccgagtgggttttagagctagaaata

SEQ ID NO: 33 - DNA, 54 bp, Artificial Sequence
  source: /note=“Description of Artificial Sequence Synthetic primer”
attattggtctcgaaacccatcgggatcatcggcaacaatctcttagtcgactc

SEQ ID NO: 34 - DNA, 22 bp, Arabidopsis thaliana
cttggcatggttatgcaatggg

SEQ ID NO: 35 - DNA, 20 bp, Artificial Sequence
  source: /note=“Description of Artificial Sequence Synthetic oligonucleotide”
cttggcatggttatgcaatg

SEQ ID NO: 36 - DNA, 26 bp, Artificial Sequence
  source: /note=“Description of Artificial Sequence Synthetic oligonucleotide”
cttggcatggttatgcaaatgggaag

SEQ ID NO: 37 - DNA, 24 bp, Artificial Sequence
  source: /note=“Description of Artificial Sequence Synthetic oligonucleotide”
cttggcatggttatgcaaatggga

SEQ ID NO: 38 - DNA, 20 bp, Oryza sativa
ggcgtgcggtttgatgatcg

SEQ ID NO: 39 - DNA, 19 bp, Artificial Sequence
  source: /note=“Description of Artificial Sequence Synthetic oligonucleotide”
ggcgtgcggtttgatgacg

SEQ ID NO: 40 - DNA, 20 bp, Oryza sativa
ggtatggttgtgcaatggga

SEQ ID NO: 41 - DNA, 19 bp, Artificial Sequence
  source: /note=“Description of Artificial Sequence Synthetic oligonucleotide”
ggtatggttgtgcaatgga

SEQ ID NO: 42 - DNA, 19 bp, Artificial Sequence
  source: /note=“Description of Artificial Sequence Synthetic oligonucleotide”
ttcatcctcgctaactgag

SEQ ID NO: 43 - DNA, 20 bp, Artificial Sequence
  source: /note=“Description of Artificial Sequence Synthetic oligonucleotide”
cttcatcctcgctaactgag

SEQ ID NO: 44 - DNA, 40 bp, Artificial Sequence
  source: /note=“Description of Artificial Sequence Synthetic primer”
gtgccgcgcggcagccatatggattacaaggaccacgacg

SEQ ID NO: 45 - DNA, 41 bp, Artificial Sequence
  source: /note=“Description of Artificial Sequence Synthetic primer”
tttgttagcagccggatcctcacttcttcttcttcgcctgc

SEQ ID NO: 46 - DNA, 42 bp, Artificial Sequence
  source: /note=“Description of Artificial Sequence Synthetic primer”
tgccgcgcggcagccatatgatggattacaaggaccacgacg

SEQ ID NO: 47 - DNA, 44 bp, Artificial Sequence
  source: /note=“Description of Artificial Sequence Synthetic primer”
tttgttagcagccggatcctcacttcttcttcttggcctgtccc

SEQ ID NO: 48 - RNA, 97 nt, Artificial Sequence
  source: /note=“Description of Artificial Sequence Synthetic oligonucleotide”
ggguaugguugugcaaugggaguuuuagagcuagaaauagcaaguuaaaauaaggcuaguccguuaucaacuugaaaaaguggcaccgagucggugc

SEQ ID NO: 49 - RNA, 97 nt, Artificial Sequence
  source: /note=“Description of Artificial Sequence Synthetic oligonucleotide”
guucauccucgcuaacuggagguuuuagagcuagaaauagcaaguuaaaauaaggcuaguccguuaucaacuugaaaaaguggcaccgagucggugc

SEQ ID NO: 50 - DNA, 19 bp, Artificial Sequence
  source: /note=“Description of Artificial Sequence Synthetic primer”
ggtcttgcgtctggttggc

SEQ ID NO: 51 - DNA, 23 bp, Artificial Sequence
  source: /note=“Description of Artificial Sequence Synthetic primer”
ccatgccaagcacatcaaacaag

SEQ ID NO: 52 - DNA, 25 bp, Artificial Sequence
  source: /note=“Description of Artificial Sequence Synthetic primer”
gcgaagaagactatgctcgtattgg

SEQ ID NO: 53 - DNA, 22 bp, Artificial Sequence
  source: /note=“Description of Artificial Sequence Synthetic primer”
cttaatcacacctttcgcagcc

SEQ ID NO: 54 - DNA, 24 bp, Oryza sativa
gggtatggttgtgcaatgggagga

SEQ ID NO: 55 - RNA, 37 nt, Artificial Sequence
  source: /note=“Description of Artificial Sequence Synthetic oligonucleotide”
ggguaugguugugcaaugggaguuuuagagcuaugcu

SEQ ID NO: 56 - RNA, 37 nt, Artificial Sequence
  source: /note=“Description of Artificial Sequence Synthetic oligonucleotide”
uggguaugguugugcaauggaguuuuagagcuaugcu

SEQ ID NO: 57 - RNA, 97 nt, Artificial Sequence
  source: /note=“Description of Artificial Sequence Synthetic oligonucleotide”
uggguaugguugugcaauggaguuuuagagcuagaaauagcaaguuaaaauaaggcuaguccguuaucaacuugaaaaaguggcaccgagucggugc

SEQ ID NO: 58 - RNA, 67 nt, Artificial Sequence
  source: /note=“Description of Artificial Sequence Synthetic oligonucleotide”
agcauagcaaguuaaaauaaggcuaguccguuaucaacuugaaaaaguggcaccgagucggugcuuu

SEQ ID NO: 59 - DNA, 20 bp, Artificial Sequence
  source: /note=“Description of Artificial Sequence Synthetic primer”
ggctaacccaggtgtcacag

SEQ ID NO: 60 - DNA, 24 bp, Oryza sativa
gttcatcctcgctaactggagagg

SEQ ID NO: 61 - RNA, 37 nt, Artificial Sequence
  source: /note=“Description of Artificial Sequence Synthetic oligonucleotide”
guucauccucgcuaacuggagguuuuagagcuaugcu

SEQ ID NO: 62 - RNA, 37 nt, Artificial Sequence
  source: /note=“Description of Artificial Sequence Synthetic oligonucleotide”
uguucauccucgcuaacugagguuuuagagcuaugcu

SEQ ID NO: 63 - RNA, 97 nt, Artificial Sequence
  source: /note=“Description of Artificial Sequence Synthetic oligonucleotide”
uguucauccucgcuaacugagguuuuagagcuagaaauagcaaguuaaaauaaggcuaguccguuaucaacuugaaaaaguggcaccgagucggugc

SEQ ID NO: 64 - DNA, 24 bp, Artificial Sequence
  source: /note=“Description of Artificial Sequence Synthetic primer”
ctgtaggcatttgaaactgcagtg

SEQ ID NO: 65 - DNA, 23 bp, Artificial Sequence
  source: /note=“Description of Artificial Sequence Synthetic primer”
gcaatcctggagttcctctgacc

SEQ ID NO: 66 - DNA, 24 bp, Oryza sativa
ccaagtacctccgcgcaatcgcgg

SEQ ID NO: 67 - RNA, 37 nt, Artificial Sequence
  source: /note=“Description of Artificial Sequence Synthetic oligonucleotide”
ccaaguaccuccgcgcaaucgguuuuagagcuaugcu

SEQ ID NO: 68 - RNA, 97 nt, Artificial Sequence
  source: /note=“Description of Artificial Sequence Synthetic oligonucleotide”
ccaaguaccuccgcgcaaucgguuuuagagcuagaaauagcaaguuaaaauaaggcuaguccguuaucaacuugaaaaaguggcaccgagucggugc

SEQ ID NO: 69 - DNA, 19 bp, Artificial Sequence
  source: /note=“Description of Artificial Sequence Synthetic primer”
catcggtaccctcctcttc

SEQ ID NO: 70 - DNA, 20 bp, Artificial Sequence
  source: /note=“Description of Artificial Sequence Synthetic primer”
atcgatcgatttggggctca

SEQ ID NO: 71 - DNA, 24 bp, Oryza sativa
gagcttagcacctggttggagggg

SEQ ID NO: 72 - RNA, 37 nt, Artificial Sequence
  source: /note=“Description of Artificial Sequence Synthetic oligonucleotide”
gagcuuagcaccugguuggagguuuuagagcuaugcu

SEQ ID NO: 73 - RNA, 97 nt, Artificial Sequence
  source: /note=“Description of Artificial Sequence Synthetic oligonucleotide”
gagcuuagcaccugguuggagguuuuagagcuagaaauagcaaguuaaaauaaggcuaguccguuaucaacuugaaaaaguggcaccgagucggugc

SEQ ID NO: 74 - DNA, 25 bp, Artificial Sequence
  source: /note=“Description of Artificial Sequence Synthetic primer”
atgggtgctgatgattatcttgtat

SEQ ID NO: 75 - DNA, 22 bp, Artificial Sequence
  source: /note=“Description of Artificial Sequence Synthetic primer”
tgaagagacatgccagccattg

SEQ ID NO: 76 - RNA, 97 nt, Artificial Sequence
  source: /note=“Description of Artificial Sequence Synthetic oligonucleotide”
gggaauggugguucagugggaguuuuagagcuagaaauagcaaguuaaaauaaggcuaguccguuaucaacuugaaaaaguggcaccgagucggugc

SEQ ID NO: 77 - RNA, 97 nt, Artificial Sequence
  source: /note=“Description of Artificial Sequence Synthetic oligonucleotide”
ugggaauggugguucaguggaguuuuagagcuagaaauagcaaguuaaaauaaggcuaguccguuaucaacuugaaaaaguggcaccgagucggugc

SEQ ID NO: 78 - DNA, 23 bp, Artificial Sequence
  source: /note=“Description of Artificial Sequence Synthetic primer”
gtggattaggagcaatgggattt

SEQ ID NO: 79 - DNA, 25 bp, Artificial Sequence
  source: /note=“Description of Artificial Sequence Synthetic primer”
ttattttagataatacaatgcctcg

SEQ ID NO: 80 - DNA, 23 bp, Homo sapiens
catggtgcacctgactcctgagg

SEQ ID NO: 81 - DNA, 23 bp, Homo sapiens
ccatggtgcatctgactctgagg

SEQ ID NO: 82 - DNA, 43 bp, Artificial Sequence
  source: /note=“Description of Artificial Sequence Synthetic primer”
gaggtgagtacggtgtgcgcttacatttgcttctgacacaact

SEQ ID NO: 83 - DNA, 45 bp, Artificial Sequence
  source: /note=“Description of Artificial Sequence Synthetic primer”
gagttggatgctggatggtctattggtctccttaaacctgtcttg

SEQ ID NO: 84 - DNA, 10 bp, Artificial Sequence
  source: /note=“Description of Artificial Sequence Synthetic oligonucleotide”
  modified_base (1)..(2): a, c, t, g, unknown or other
  modified_base (5)..(8): a, c, t, g, unknown or other
nnaannnngg

SEQ ID NO: 85 - DNA, 10 bp, Artificial Sequence
  source: /note=“Description of Artificial Sequence Synthetic oligonucleotide”
  modified_base (1)..(2): a, c, t, g, unknown or other
  modified_base (5)..(8): a, c, t, g, unknown or other
nnatnnnngg

SEQ ID NO: 86 - DNA, 10 bp, Artificial Sequence
  source: /note=“Description of Artificial Sequence Synthetic oligonucleotide”
  modified_base (1)..(2): a, c, t, g, unknown or other
  modified_base (5)..(8): a, c, t, g, unknown or other
nnacnnnngg

SEQ ID NO: 87 - DNA, 10 bp, Artificial Sequence
  source: /note=“Description of Artificial Sequence Synthetic oligonucleotide”
  modified_base (1)..(2): a, c, t, g, unknown or other
  modified_base (5)..(8): a, c, t, g, unknown or other
nnagnnnngg

SEQ ID NO: 88 - DNA, 67 bp, Arabidopsis thaliana
ttccagtgaaggtacttttattaaacaaccagcatcttggcatggttatgcaatgggaagatcggtt

SEQ ID NO: 89 - DNA, 67 bp, Arabidopsis thaliana
aaccgatcttcccattgcataaccatgccaagatgctggttgtttaataaaagtaccttcactggaa

SEQ ID NO: 90 - protein, 23 aa, Arabidopsis thaliana
LPVKVLLLNNQHLGMVMQWEDRF

SEQ ID NO: 91 - DNA, 18 bp, Artificial Sequence
  source: /note=“Description of Artificial Sequence Synthetic oligonucleotide”
gcatggttatgcaatgga

SEQ ID NO: 92 - DNA, 20 bp, Artificial Sequence
  source: /note=“Description of Artificial Sequence Synthetic oligonucleotide”
ggcatggttatgcaattgga

SEQ ID NO: 93 - DNA, 71 bp, Arabidopsis thaliana
ggatgtgatttgtccgcaccaagaacatgtgttgccgatgatcccgagtggtggcactttcaacgatgtca

SEQ ID NO: 94 - DNA, 71 bp, Arabidopsis thaliana
tgacatcgttgaaagtgccaccactcgggatcatcggcaacacatgttcttggtgcggacaaatcacatcc

SEQ ID NO: 95 - protein, 23 aa, Arabidopsis thaliana
DVICPHQEHVLPMIPSGGTFNDV

SEQ ID NO: 96 - DNA, 18 bp, Artificial Sequence
  source: /note=“Description of Artificial Sequence Synthetic oligonucleotide”
tgccgatgatcccgatgg

SEQ ID NO: 97 - DNA, 20 bp, Artificial Sequence
  source: /note=“Description of Artificial Sequence Synthetic oligonucleotide”
ttgccgatgatcccgaatgg

SEQ ID NO: 98 - DNA, 22 bp, Artificial Sequence
  source: /note=“Description of Artificial Sequence Synthetic oligonucleotide”
gcatggttatgcaattggaaga

SEQ ID NO: 99 - DNA, 28 bp, Artificial Sequence
  source: /note=“Description of Artificial Sequence Synthetic oligonucleotide”
gattggcatggttatgcaatgggagttt

SEQ ID NO: 100 - DNA, 27 bp, Artificial Sequence
  source: /note=“Description of Artificial Sequence Synthetic oligonucleotide”
gattggcatggttatgcaatggagttt

SEQ ID NO: 101 - DNA, 27 bp, Arabidopsis thaliana
cttggcatggttatgcaatgggaagat

SEQ ID NO: 102 - DNA, 27 bp, Arabidopsis thaliana
atcttcccattgcataaccatgccaag

SEQ ID NO: 103 - protein, 9 aa, Arabidopsis thaliana
LGMVMQWED

SEQ ID NO: 104 - DNA, 27 bp, Artificial Sequence
  source: /note=“Description of Artificial Sequence Synthetic oligonucleotide”
cttggcatggttatgcaatggaagatc

SEQ ID NO: 105 - DNA, 27 bp, Artificial Sequence
  source: /note=“Description of Artificial Sequence Synthetic oligonucleotide”
gatcttccattgcataaccatgccaag

SEQ ID NO: 106 - protein, 9 aa, Artificial Sequence
  source: /note=“Description of Artificial Sequence Synthetic peptide”
LGMVMQWKI

SEQ ID NO: 107 - DNA, 27 bp, Artificial Sequence
  source: /note=“Description of Artificial Sequence Synthetic oligonucleotide”
cttggcatggttatgcaattggaagat

SEQ ID NO: 108 - DNA, 27 bp, Artificial Sequence
  source: /note=“Description of Artificial Sequence Synthetic oligonucleotide”
atcttccaattgcataaccatgccaag

SEQ ID NO: 109 - protein, 9 aa, Artificial Sequence
  source: /note=“Description of Artificial Sequence Synthetic peptide”
LGMVMQLED

SEQ ID NO: 110 - DNA, 14 bp, Artificial Sequence
  source: /note=“Description of Artificial Sequence Synthetic oligonucleotide”
atgcaattggaaga

SEQ ID NO: 111 - DNA, 14 bp, Artificial Sequence
  source: /note=“Description of Artificial Sequence Synthetic oligonucleotide”
atgcaatkggaaga

SEQ ID NO: 112 - DNA, 14 bp, Artificial Sequence
  source: /note=“Description of Artificial Sequence Synthetic oligonucleotide”
atgcaawtggaaga

SEQ ID NO: 113 - DNA, 28 bp, Artificial Sequence
  source: /note=“Description of Artificial Sequence Synthetic oligonucleotide”
gattttgccgatgatcccgagtgggttt

SEQ ID NO: 114 - DNA, 28 bp, Artificial Sequence
  source: /note=“Description of Artificial Sequence Synthetic oligonucleotide”
gattgttgccgatgatcccgatgggttt

SEQ ID NO: 115 - DNA, 30 bp, Arabidopsis thaliana
ttgccgatgatcccgagtggtggcactttc

SEQ ID NO: 116 - DNA, 30 bp, Arabidopsis thaliana
gaaagtgccaccactcgggatcatcggcaa

SEQ ID NO: 117 - protein, 10 aa, Arabidopsis thaliana
LPMIPSGGTF

SEQ ID NO: 118 - DNA, 29 bp, Artificial Sequence
  source: /note=“Description of Artificial Sequence Synthetic oligonucleotide”
ttgccgatgatcccgatggtggcactttc

SEQ ID NO: 119 - DNA, 29 bp, Artificial Sequence
  source: /note=“Description of Artificial Sequence Synthetic oligonucleotide”
gaaagtgccaccatcgggatcatcggcaa

SEQ ID NO: 120 - protein, 10 aa, Artificial Sequence
  source: /note=“Description of Artificial Sequence Synthetic peptide”
LPMIPMVALS

SEQ ID NO: 121 - DNA, 24 bp, Artificial Sequence
  source: /note=“Description of Artificial Sequence Synthetic oligonucleotide”
ttgccgatgatcccgaatggtggc

SEQ ID NO: 122 - DNA, 24 bp, Artificial Sequence
  source: /note=“Description of Artificial Sequence Synthetic oligonucleotide”
gccaccattcgggatcatcggcaa

SEQ ID NO: 123 - protein, 8 aa, Artificial Sequence
  source: /note=“Description of Artificial Sequence Synthetic peptide”
LPMIPNGG

SEQ ID NO: 124 - DNA, 12 bp, Artificial Sequence
  source: /note=“Description of Artificial Sequence Synthetic oligonucleotide”
ccgagagrtggc

SEQ ID NO: 125 - DNA, 12 bp, Artificial Sequence
  source: /note=“Description of Artificial Sequence Synthetic oligonucleotide”
ccgarwggtggc

SEQ ID NO: 126 - DNA, 28 bp, Artificial Sequence
  source: /note=“Description of Artificial Sequence Synthetic oligonucleotide”
gattcttggcatggttatgcaatggttt

SEQ ID NO: 127 - DNA, 28 bp, Artificial Sequence
  source: /note=“Description of Artificial Sequence Synthetic oligonucleotide”
gattgcatggttatgcaaatgggagttt

SEQ ID NO: 128 - DNA, 27 bp, Artificial Sequence
  source: /note=“Description of Artificial Sequence Synthetic oligonucleotide”
cttggcatggttatgcaaatgggaaga

SEQ ID NO: 129 - DNA, 27 bp, Artificial Sequence
  source: /note=“Description of Artificial Sequence Synthetic oligonucleotide”
tcttcccatttgcataaccatgccaag

SEQ ID NO: 130 - protein, 9 aa, Artificial Sequence
  source: /note=“Description of Artificial Sequence Synthetic peptide”
LGMVMQMGR

SEQ ID NO: 131 - DNA, 26 bp, Artificial Sequence
  source: /note=“Description of Artificial Sequence Synthetic oligonucleotide”
cttggcatggttatgcaaatggaaga

SEQ ID NO: 132 - DNA, 26 bp, Artificial Sequence
  source: /note=“Description of Artificial Sequence Synthetic oligonucleotide”
tcttccatttgcataaccatgccaag

SEQ ID NO: 133 - protein, 9 aa, Artificial Sequence
  source: /note=“Description of Artificial Sequence Synthetic peptide”
LGMVMQMED

SEQ ID NO: 134 - DNA, 28 bp, Artificial Sequence
  source: /note=“Description of Artificial Sequence Synthetic oligonucleotide”
ggcgttcatcctcgctaactggaggttt

SEQ ID NO: 135 - DNA, 28 bp, Artificial Sequence
  source: /note=“Description of Artificial Sequence Synthetic oligonucleotide”
tgcagttcatcctcgctaactgaggttt

SEQ ID NO: 136 - DNA, 30 bp, Oryza sativa
ctgttcatcctcgctaactggagaggcttc

SEQ ID NO: 137 - DNA, 30 bp, Oryza sativa
gaagcctctccagttagcgaggatgaacag

SEQ ID NO: 138 - protein, 10 aa, Oryza sativa
LFILANWRGF

SEQ ID NO: 139 - DNA, 29 bp, Artificial Sequence
  source: /note=“Description of Artificial Sequence Synthetic oligonucleotide”
ctgttcatcctcgctaactgagaggcttc

SEQ ID NO: 140 - DNA, 29 bp, Artificial Sequence
  source: /note=“Description of Artificial Sequence Synthetic oligonucleotide”
gaagcctctcagttagcgaggatgaacag

SEQ ID NO: 141 - protein, 6 aa, Artificial Sequence
  source: /note=“Description of Artificial Sequence Synthetic peptide”
LFILAN

SEQ ID NO: 142 - DNA, 20 bp, Artificial Sequence
  source: /note=“Description of Artificial Sequence Synthetic oligonucleotide”
gctaacttgagaggcttctc

SEQ ID NO: 143 - DNA, 20 bp, Artificial Sequence
  source: /note=“Description of Artificial Sequence Synthetic oligonucleotide”
gagaagcctctcaagttagc

SEQ ID NO: 144 - protein, 7 aa, Artificial Sequence
  source: /note=“Description of Artificial Sequence Synthetic peptide”
ANLRGFS

SEQ ID NO: 145 - DNA, 14 bp, Artificial Sequence
  source: /note=“Description of Artificial Sequence Synthetic oligonucleotide”
gctaacttgagagg

SEQ ID NO: 146 - DNA, 14 bp, Artificial Sequence
  source: /note=“Description of Artificial Sequence Synthetic oligonucleotide”
gctaactkgagagg

SEQ ID NO: 147 - DNA, 14 bp, Artificial Sequence
  source: /note=“Description of Artificial Sequence Synthetic oligonucleotide”
gctaactgtagagg

SEQ ID NO: 148 - DNA, 21 bp, Oryza sativa
tgcaatgggaggataggtttt

SEQ ID NO: 149 - DNA, 21 bp, Artificial Sequence
  source: /note=“Description of Artificial Sequence Synthetic oligonucleotide”
  modified_base (7)..(7): a, c, t, g, unknown or other
tgcaatnggaggataggtttt

SEQ ID NO: 150 - DNA, 21 bp, Oryza sativa
ctcgctaactggagaggcttc

SEQ ID NO: 151 - DNA, 21 bp, Artificial Sequence
  source: /note=“Description of Artificial Sequence Synthetic oligonucleotide”
ctcgctaacttgagaggcttc

SEQ ID NO: 152 - DNA, 25 bp, Artificial Sequence
  source: /note=“Description of Artificial Sequence Synthetic oligonucleotide”
ccaagtacctccgcgcaaatcgcgg

SEQ ID NO: 153 - DNA, 22 bp, Oryza sativa
aaaccccctccaaccaggtgct

SEQ ID NO: 154 - DNA, 21 bp, Artificial Sequence
  source: /note=“Description of Artificial Sequence Synthetic oligonucleotide”
aaaccccctcaaccaggtgct

SEQ ID NO: 155 - DNA, 20 bp, Artificial Sequence
  source: /note=“Description of Artificial Sequence Synthetic oligonucleotide”
catggtgcatctgactcctg

SEQ ID NO: 156 - DNA, 20 bp, Artificial Sequence
  source: /note=“Description of Artificial Sequence Synthetic oligonucleotide”
ccatggtgcatctgactctg

SEQ ID NO: 157 - DNA, 20 bp, Artificial Sequence
  source: /note=“Description of Artificial Sequence Synthetic oligonucleotide”
ggctcccatcacatcaacgg